SEQUENCE LISTING (48 sequences)

SEQ ID NO: 1 - DNA, 21 bp, synthetic construct
  source 1..21: mol_type = other DNA
actggtggtccatgaaaaagc

SEQ ID NO: 2 - DNA, 20 bp, synthetic construct
  source 1..20: mol_type = other DNA
aacgacttccagcgtttcct

SEQ ID NO: 3 - DNA, 20 bp, synthetic construct
  source 1..20: mol_type = other DNA
cggatgaagagaggcatgtt

SEQ ID NO: 4 - DNA, 20 bp, synthetic construct
  source 1..20: mol_type = other DNA
caatcacaccacaagccaag

SEQ ID NO: 5 - DNA, 25 bp, synthetic construct
  source 1..25: mol_type = other DNA
gctcttctggacaagtacaatgctg

SEQ ID NO: 6 - DNA, 24 bp, synthetic construct
  source 1..24: mol_type = other DNA
ttacacggatgaacgctaagcttc

SEQ ID NO: 7 - DNA, 18 bp, synthetic construct
  source 1..18: mol_type = other DNA
ggcagctatgtgagagcc

SEQ ID NO: 8 - DNA, 17 bp, synthetic construct
  source 1..17: mol_type = other DNA
ctgacgtccaccctgac

SEQ ID NO: 9 - DNA, 20 bp, synthetic construct
  source 1..20: mol_type = other DNA
caaatcccaccttgaacaca

SEQ ID NO: 10 - DNA, 20 bp, synthetic construct
  source 1..20: mol_type = other DNA
cgactgactaatggcagcag

SEQ ID NO: 11 - DNA, 20 bp, synthetic construct
  source 1..20: mol_type = other DNA
caaatcccaccttgaacaca

SEQ ID NO: 12 - DNA, 20 bp, synthetic construct
  source 1..20: mol_type = other DNA
cgactgactaatggcagcag

SEQ ID NO: 13 - DNA, 24 bp, synthetic construct
  source 1..24: mol_type = other DNA
atcgctctgtggatgactgagtac

SEQ ID NO: 14 - DNA, 24 bp, synthetic construct
  source 1..24: mol_type = other DNA
agagacagccaggagaaatcaaac

SEQ ID NO: 15 - DNA, 22 bp, synthetic construct
  source 1..22: mol_type = other DNA
catgctgatgtctctggaatct

SEQ ID NO: 16 - DNA, 20 bp, synthetic construct
  source 1..20: mol_type = other DNA
gatgtgatgcctctgcgaag

SEQ ID NO: 17 - DNA, 20 bp, synthetic construct
  source 1..20: mol_type = other DNA
agttcccgggtgctgtctat

SEQ ID NO: 18 - DNA, 20 bp, synthetic construct
  source 1..20: mol_type = other DNA
gccatggtctttctgctcac

SEQ ID NO: 19 - DNA, 20 bp, synthetic construct
  source 1..20: mol_type = other DNA
agttcccgggtgctgtctat

SEQ ID NO: 20 - DNA, 20 bp, synthetic construct
  source 1..20: mol_type = other DNA
gccatggtctttctgctcac

SEQ ID NO: 21 - DNA, 20 bp, synthetic construct
  source 1..20: mol_type = other DNA
agagctacgagctgcctgac

SEQ ID NO: 22 - DNA, 20 bp, synthetic construct
  source 1..20: mol_type = other DNA
agcactgtgttggcgtacag

SEQ ID NO: 23 - DNA, 19 bp, synthetic construct
  source 1..19: mol_type = other DNA
atctggcaccacaccttct

SEQ ID NO: 24 - DNA, 20 bp, synthetic construct
  source 1..20: mol_type = other DNA
agcacagcctggatagcaac

SEQ ID NO: 25 - DNA, 20 bp, synthetic construct
  source 1..20: mol_type = other DNA
cggatgaagagaggcatgtt

SEQ ID NO: 26 - DNA, 20 bp, synthetic construct
  source 1..20: mol_type = other DNA
caatcacaccacaagccaag

SEQ ID NO: 27 - DNA, 25 bp, synthetic construct
  source 1..25: mol_type = other DNA
gctcttctggacaagtacaatgctg

SEQ ID NO: 28 - DNA, 24 bp, synthetic construct
  source 1..24: mol_type = other DNA
ttacacggatgaacgctaagcttc

SEQ ID NO: 29 - DNA, 18 bp, synthetic construct
  source 1..18: mol_type = other DNA
ggcagctatgtgagagcc

SEQ ID NO: 30 - DNA, 17 bp, synthetic construct
  source 1..17: mol_type = other DNA
ctgacgtccaccctgac

SEQ ID NO: 31 - DNA, 20 bp, synthetic construct
  source 1..20: mol_type = other DNA
tggcgactccagtgtgcctg

SEQ ID NO: 32 - DNA, 21 bp, synthetic construct
  source 1..21: mol_type = other DNA
aggcactgtccaccacctgct

SEQ ID NO: 33 - DNA, 24 bp, synthetic construct
  source 1..24: mol_type = other DNA
tggatggagaggaagctttctaat

SEQ ID NO: 34 - DNA, 22 bp, synthetic construct
  source 1..22: mol_type = other DNA
cacaccagattttccagctttc

SEQ ID NO: 35 - DNA, 20 bp, synthetic construct
  source 1..20: mol_type = other DNA
caaatcccaccttgaacaca

SEQ ID NO: 36 - DNA, 20 bp, synthetic construct
  source 1..20: mol_type = other DNA
cgactgactaatggcagcag

SEQ ID NO: 37 - DNA, 20 bp, synthetic construct
  source 1..20: mol_type = other DNA
caatggcggcggtcctataa

SEQ ID NO: 38 - DNA, 20 bp, synthetic construct
  source 1..20: mol_type = other DNA
ggagaatggacgtgctgtga

SEQ ID NO: 39 - DNA, 24 bp, synthetic construct
  source 1..24: mol_type = other DNA
atcgctctgtggatgactgagtac

SEQ ID NO: 40 - DNA, 24 bp, synthetic construct
  source 1..24: mol_type = other DNA
agagacagccaggagaaatcaaac

SEQ ID NO: 41 - DNA, 22 bp, synthetic construct
  source 1..22: mol_type = other DNA
catgctgatgtctctggaatct

SEQ ID NO: 42 - DNA, 20 bp, synthetic construct
  source 1..20: mol_type = other DNA
gatgtgatgcctctgcgaag

SEQ ID NO: 43 - DNA, 20 bp, synthetic construct
  source 1..20: mol_type = other DNA
agttcccgggtgctgtctat

SEQ ID NO: 44 - DNA, 20 bp, synthetic construct
  source 1..20: mol_type = other DNA
gccatggtctttctgctcac

SEQ ID NO: 45 - DNA, 20 bp, synthetic construct
  source 1..20: mol_type = other DNA
ctctgggcgccgcgagatac

SEQ ID NO: 46 - DNA, 20 bp, synthetic construct
  source 1..20: mol_type = other DNA
caactaccaccgcccagccc

SEQ ID NO: 47 - DNA, 20 bp, synthetic construct
  source 1..20: mol_type = other DNA
agagctacgagctgcctgac

SEQ ID NO: 48 - DNA, 20 bp, synthetic construct
  source 1..20: mol_type = other DNA
agcactgtgttggcgtacag

(19) United States
(12) Patent Application Publication
(10) Pub. No.: US 2025/0275938 A1
(43) Pub. Date: Sep. 4, 2025

(54) COMPOSITIONS AND METHODS FOR TREATMENT OF CANCER

(71) Applicant: SCIROSBIO (FZC), Sharjah (AE)

(72) Inventors: Madhu SUBRAMANI, Dubai (AE); Anitha T. SIVASUBRAMANIAN, Puducherry (IN); Shreyas S. KUDUVALLI, Bengaluru, Karnataka (IN); Daisy S. PRECILLA, Salem, Tamilnadu (IN)

(21) Appl. No.: 18/860,360

(22) PCT Filed: Apr. 27, 2023
(86) PCT No.: PCT/IB2023/054358

§ 371 Date: Oct. 25, 2024

Related U.S. Application Data
(60) Provisional application No. 63/363,765, filed on Apr. 28, 2022.

(51) Int. Cl. A61K 31/353; A61K 31/155; A61K 31/44; A61K 31/495; A61K 31/513; A61K 31/704; A61K 33/243; A61P 35/00

(52) U.S. Cl. CPC A61K 31/353; A61K 31/155; A61K 31/44; A61K 31/495; A61K 31/513; A61K 31/704; A61K 33/243; A61P 35/00

(57) ABSTRACT

The invention provides pharmaceutical compositions comprising metformin and epigallocatechin gallate. The pharmaceutical compositions further comprise a chemotherapeutic agent. The invention also provides kits comprising a composition comprising metformin, a composition comprising epigallocatechin gallate, and a composition comprising a chemotherapeutic agent. The invention further methods of inhibiting growth or promoting apoptosis of a cancer cell comprising contacting the cancer cell with an effective amount of a chemotherapeutic agent, metformin, and epigallocatechin gallate. Also provided are methods of treating or ameliorating cancer in a subject in need thereof, comprising administering to the subject a therapeutically effective amount of a chemotherapeutic agent, metformin, and epigallocatechin gallate.

### FIELD OF THE INVENTION

The invention relates to compositions comprising metformin and epigallocatechin gallate, and optionally further comprising a chemotherapeutic agent. The invention further provides methods of treating cancer with a combination of metformin, epigallocatechin gallate, and a chemotherapeutic agent. The invention further provides methods of treating glioblastoma multiforme, ovarian cancer, pancreatic cancer, and hepatocellular carcinoma by administering a combination of metformin, epigallocatechin gallate, and a chemotherapeutic agent, resulting in synergistic treatment compared to each agent alone.

### BACKGROUND

Glioblastoma (GBM) is the most common and heterogeneous malignant brain tumor in adults, accounting for more than 50% of the total glioma cases. It is one of the most lethal tumors and is a challenging disease to treat and regarded as a major unmet medical need. Current multi-modal treatment options include surgical resection followed by radiotherapy and chemotherapy employing temozolomide (TMZ). Unfortunately, despite aggressive therapeutic treatment modalities, the prognosis for GMB patients remains poor, with a median overall survival of 12-14 months. While numerous therapeutic regimens have been investigated for treatment of GBM, such as combination therapy with various chemotherapeutic agents, targeted drug therapy, and immunotherapy with CAR-T cells and immunoconjugates to target check-point inhibitors, these approaches have exhibited limited efficacy.

TMZ is known for its anti-tumor effects in GBM as well as other cancers. For example, TMZ has exhibited limited efficacy in treatment of melanoma, with reported clinical responses in 17-21% of treated patients (Newlands E S, et al., Br J Cancer 65(2):287-2981 (1992); Bleehen N M, et al., Journal of Clinical Oncology, 13(4):910-913 (1995)). In addition to melanoma, treatment of astrocytoma, lung cancer, breast cancer, testicular cancer, colon and rectal cancers, carcinomas, sarcomas, lymphomas, leukemias, and mycosis fungoides has also been reported.

However, the efficacy of TMZ treatment of these and other cancers remains low, and there exists an unmet need for effective treatment of GBM.

Primary liver cancer is the fifth roost frequently diagnosed cancer globally and the second leading cause of cancer death. Liver cancers are malignant tumors that grow on the surface or inside the liver. They are formed from either the liver itself or from structures within the liver, including blood vessels or the bile duct.

The roost frequent liver cancer, accounting for approximately 75% of all primary liver cancers, is hepatocellular carcinoma (HCC). HCC is a cancer formed by liver cells, known as hepatocytes that become malignant.

Sorafenib (marketed as NEXAVAR®), is an FDA-approved drug for patients with advanced primary liver cancer. It is a small molecule interacting with multiple intracellular and cell surface kinases including the Raf/Mek/Erk pathway. By inhibiting these kinases, genetic transcription involving cell proliferation and angiogenesis is inhibited, with the observation that hypoxia in solid tumor tissues may be increased due to the treatment reducing blood supply to the tumor. However, even with the development of drugs like sorafenib, the current treatment options for liver cancer are insufficient due to its limited effectiveness and severe toxicity.

Ovarian cancer presents non-specific symptoms and it has the highest mortality of all gynecological cancers. About 19,880 new ovarian cancer cases and 12,810 deaths are expected in the United States in 2022.

Surgery and platinum-based adjuvant chemotherapy, such as cisplatin (CIS), are the most common treatments for ovarian cancer. Unfortunately, over 70% of women develop chemoresistance. The exact mechanism of CIS resistance is not known, however, evidence indicates that activation of the oncogenic transcription factor c-MYC is involved in drug resistance.

### SUMMARY OF THE INVENTION

In embodiments, the present invention relates to pharmaceutical compositions comprising metformin and epigallocatechin gallate (ECGC). In embodiments, the pharmaceutical composition further comprises a chemotherapeutic agent.

In embodiments, the chemotherapeutic agent is TMZ, sorafenib, cisplatin, topotecan, pegylated liposomal doxorubicin (PLD), thiotepa, cyclosphosphamide, busulfan, improsulfan, piposulfan, aziridines, benzodopa, carboquone, meturedopa, uredopa, ethylenimines, altretamine, triethylenemelamine, trietylenephosphoramide, triethiylenethiophosphoramide, trimethylolomelamine, acetogenins, bullatacin, bullatacinone, a camptothecin, bryostatin; callystatin; CC-1065, adozelesin, carzelesin, bizelesin, cryptophycin 1, cryptophycin 8, dolastatin; duocarmycin, KW-2189, CB1-TM1, eleutherobin, pancratistatin, a sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, nitrosureas, carmustine, chlorozotocin, fotemustine, lomustine, nimustine, ranimnustine, enediyne antibiotics, calicheamicins, dynemicin A; bisphosphonates, clodronate, an esperamicin, neocarzinostatin, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin, deoxydoxorubicin, epirubicin, esorubicin, idarubicin, marcellomycin, mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; methotrexate, 5-fluorouracil (5-FU); folic acid analogues, denopterin, pteropterin, trimetrexate; purine analogs, fludarabine, 6-mercaptopurine, thiamiprine, thioguanine, pyrimidine analogs, ancitabine, azacytidine, 6-azauridine, carmofur, cytarabine, dideoxyuridine, doxifluridine, enocitabine, floxuridine; androgens, calusterone, dromostanolone propionate, epitiostanol, mepitiostane, testolactone; anti-adrenals, aminoglutethimide, mitotane, trilostane; folic acid replenishers, frolinic acid, aceglatone, aldophosphamide glycoside, aminolevulinic acid, eniluracil, amsacrine, bestrabucil, bisantrene, edatrexate, defofamine, demecolcine, diaziquone, elformithine, elliptinium acetate, an epothilone, etoglucid, gallium nitrate, hydroxyurea, lentinan, lonidainine, maytansinoids, maytansine, ansamitocins, mitoguazone, mitoxantrone, mopidanmol; nitraerine, pentostatin, phenamet, pirarubicin, losoxantrone, podophyllinic acid, 2-ethylhydrazide, procarbazine, razoxane, rhizoxin, sizofuran, spirogermanium, tenuazonic acid, triaziquone; 2,2′,2″-trichlorotriethylamine, trichothecenes, T-2 toxin, verracurin A, roridin A, anguidine, urethan, vindesine, dacarbazine, mannomustine, mitobronitol, mitolactol, pipobroman, gacytosine, arabinoside (“Ara-C”), cyclophosphamide, thiotepa, taxoids, paclitaxel, an albumin-engineered nanoparticle formulation of paclitaxel, docetaxel, chlorambucil, gemcitabine, 6-thioguanine, mercaptopurine, methotrexate, platinum analogs, oxaliplatin, carboplatin, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluoromethylornithine (DFMO), retinoic acid, capecitabine, combretastatin, leucovorin (LV), oxaliplatin, lapatinib, inhibitors of PKC-alpha, Raf, H-Ras, EGFR, erlotinib, VEGF inhibitors and pharmaceutically acceptable salts, acids or derivatives of any of the above.

In embodiments, the chemotherapeutic agent is selected from the group consisting of temozolomide, sorafenib, cisplatin, 5-fluorouracil and doxorubicin.

In embodiments, the chemotherapeutic agent is TMZ. In embodiments, the chemotherapeutic agent is sorafenib. In embodiments, the chemotherapeutic agent is cisplatin. In embodiments, the chemotherapeutic agent is 5-fluorouracil. In embodiments, the chemotherapeutic agent is doxorubicin.

In embodiments, the composition is formulated for parenteral, intrathecal, topical, buccal, oral, sublingual, subcutaneous, intraperitoneal, intrapulmonary, nasal, inhalation, or intralesional/intratumoral administration.

In embodiments, the composition is in the form of a tablet, pill, capsule, sachet, effervescent, dragee, lozenge, cream, or a solution suitable for injection.

In embodiments, the amount of the chemotherapeutic agent, metformin, and EGCG is from about 0.001 mg to about 500 mg. In embodiments, the amount of the chemotherapeutic agent, metformin, and EGCG is from about 0.01 mg/kg to about 500 mg/kg for a 70 kg human subject.

In embodiments, the present invention relates to a kit comprising one or more compositions comprising a chemotherapeutic agent, metformin, and EGCG.

In embodiments, the kit comprises a chemotherapeutic agent which is TMZ, sorafenib, cisplatin, topotecan, pegylated liposomal doxorubicin (PLD), thiotepa, cyclosphosphamide, busulfan, improsulfan, piposulfan, aziridines, benzodopa, carboquone, meturedopa, uredopa, ethylenimines, altretamine, triethylenemelamine, trietylenephosphoramide, triethiylenethiophosphoramide, trimethylolomelamine, acetogenins, bullatacin, bullatacinone, a camptothecin, bryostatin; callystatin; CC-1065, adozelesin, carzelesin, bizelesin, cryptophycin 1, cryptophycin 8, dolastatin; duocarmycin, KW-2189, CB1-TM1, eleutherobin, pancratistatin, a sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, nitrosureas, carmustine, chlorozotocin, fotemustine, lomustine, nimustine, ranimnustine, enediyne antibiotics, calicheamicins, dynemicin A; bisphosphonates, clodronate, an esperamicin, neocarzinostatin, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin, deoxydoxorubicin, epirubicin, esorubicin, idarubicin, marcellomycin, mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; methotrexate, 5-fluorouracil (5-FU); folic acid analogues, denopterin, pteropterin, trimetrexate; purine analogs, fludarabine, 6-mercaptopurine, thiamiprine, thioguanine, pyrimidine analogs, ancitabine, azacytidine, 6-azauridine, carmofur, cytarabine, dideoxyuridine, doxifluridine, enocitabine, floxuridine; androgens, calusterone, dromostanolone propionate, epitiostanol, mepitiostane, testolactone; anti-adrenals, aminoglutethimide, mitotane, trilostane; folic acid replenishers, frolinic acid, aceglatone, aldophosphamide glycoside, aminolevulinic acid, eniluracil, amsacrine, bestrabucil, bisantrene, edatrexate, defofamine, demecolcine, diaziquone, elformithine, elliptinium acetate, an epothilone, etoglucid, gallium nitrate, hydroxyurea, lentinan, lonidainine, maytansinoids, maytansine, ansamitocins, mitoguazone, mitoxantrone, mopidanmol; nitraerine, pentostatin, phenamet, pirarubicin, losoxantrone, podophyllinic acid, 2-ethylhydrazide, procarbazine, razoxane, rhizoxin, sizofuran, spirogermanium, tenuazonic acid, triaziquone; 2,2′,2″-trichlorotriethylamine, trichothecenes, T-2 toxin, verracurin A, roridin A, anguidine, urethan, vindesine, dacarbazine, mannomustine, mitobronitol, mitolactol, pipobroman, gacytosine, arabinoside (“Ara-C”), cyclophosphamide, thiotepa, taxoids, paclitaxel, an albumin-engineered nanoparticle formulation of paclitaxel, docetaxel, chlorambucil, gemcitabine, 6-thioguanine, mercaptopurine, methotrexate, platinum analogs, oxaliplatin, carboplatin, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluoromethylornithine (DFMO), retinoic acid, capecitabine, combretastatin, leucovorin (LV), oxaliplatin, lapatinib, inhibitors of PKC-alpha, Raf, H-Ras, EGFR, erlotinib, VEGF inhibitors and pharmaceutically acceptable salts, acids or derivatives of any of the above.

In embodiments, the chemotherapeutic agent is selected from the group consisting of temozolomide, sorafenib, cisplatin, 5-fluorouracil and doxorubicin.

In embodiments, the chemotherapeutic agent is TMZ. In embodiments, the chemotherapeutic agent is sorafenib. In embodiments, the chemotherapeutic agent is cisplatin. In embodiments, the chemotherapeutic agent is 5-fluorouracil. In embodiments, the chemotherapeutic agent is doxorubicin.

In embodiments, each composition in the kit is in the form of a tablet, pill, capsule, sachet, effervescent, dragee, lozenge, cream, or a solution suitable for injection.

In embodiments, each of the chemotherapeutic agent, metformin, and EGCG are in the same composition.

In embodiments, the chemotherapeutic agent, metformin, and EGCG are in separate compositions.

In embodiments, the chemotherapeutic agent is in a separate composition, and the metformin and EGCG are in the same composition.

In embodiments, the amount of each of the chemotherapeutic agent, metformin, and EGCG is from about 0.001 mg to about 500 mg In embodiments, the amount of each of the chemotherapeutic agent, metformin, and EGCG is from about 0.01 mg/kg to about 500 mg/kg for a 70 kg human subject.

In embodiments, the present invention relates to methods for inhibiting growth or promoting apoptosis of a cancer cell. In embodiments, the methods comprise contacting the cancer cell with an effective amount of a chemotherapeutic agent, an effective amount of metformin (MET), and an effective amount of EGCG, thereby inhibiting the growth of the cancer cell or promoting apoptosis of the cancer cell.

In embodiments, the cancer is selected from the group consisting of, a bone cancer cell, a brain cancer cell, a breast cancer cell, colon cancer cell, a melanoma cell, a pancreatic cancer cell, a hepatocellular carcinoma cell, an ovarian cancer cell, a head and neck cancer cell, a lung cancer cell, a renal carcinoma cell, a lymphoma cell, a leukemia cell, a sarcoma, and a carcinoma.

In embodiments, the cancer is selected from the group consisting of a brain cancer cell, a breast cancer cell, colon cancer cell, a melanoma cell, a pancreatic cancer cell, a hepatocellular carcinoma cell, an ovarian cancer cell, a head and neck cancer cell, a lung cancer cell, a renal carcinoma cell, a lymphoma cell, a leukemia cell, a sarcoma, and a carcinoma.

In embodiments, the cancer cell is a brain cancer cell. In embodiments, the brain cancer cell is a glioma cell. In embodiments, the glioma cell is a glioblastoma cell. In embodiments, the cancer cell is a pancreatic cancer cell. In embodiments, the cancer cell is a hepatocellular carcinoma cell. In embodiments, the cancer cell is an ovarian cancer cell. In embodiments, the cancer cell is a colon cancer cell. In embodiments, the cancer cell is a breast cancer cell. In embodiments, the cancer cell is a bone cancer cell.

In embodiments, the cancer cell is a human cancer cell.

In embodiments, the effective amount of each of the chemotherapeutic agent, MET, and EGCG is about 0.01 mg to about 500 mg/kg body weight. In embodiments, the effective amount of each of the chemotherapeutic agent, MET, and EGCG is about 0.01 mg/kg to about 500 mg/kg, preferably about 0.1 mg/kg to about 500 mg/kg for a 70 kg human subject.

In embodiments, the present invention relates to methods for treatment or amelioration of a cancer. In embodiments, the methods comprise administering to a subject in need thereof a therapeutically effective amount of a chemotherapeutic agent, a therapeutically effective amount of MET, and a therapeutically effective amount of EGCG, thereby treating or ameliorating the cancer in the subject.

In embodiments, the cancer is selected from the group consisting of bone cancer, brain cancer, breast cancer, colon cancer, colorectal cancer, melanoma, pancreatic cancer, hepatocellular carcinoma, ovarian cancer, head and neck cancer, lung cancer, renal cancer, a lymphoma, a leukemia, a sarcoma, and a carcinoma.

In embodiments, the cancer is selected from the group consisting of brain cancer, breast cancer, colon cancer, colorectal cancer, melanoma, pancreatic cancer, hepatocellular carcinoma, ovarian cancer, head and neck cancer, lung cancer, renal cancer, a lymphoma, a leukemia, a sarcoma, and a carcinoma.

In embodiments, cancer is a brain cancer. In embodiments, the brain cancer is a glioma. In embodiments, the glioma is GBM. In embodiments, the cancer is a hepatocellular carcinoma. In embodiments, the cancer is an ovarian cancer. In embodiments, the cancer is a pancreatic cancer. In embodiments, the cancer is colon cancer. In embodiments, the cancer is breast cancer. In embodiments, the cancer is bone cancer.

In embodiments, the subject is a human.

In embodiments, the therapeutically effective amount of the chemotherapeutic agent is about 0.1 to about 200 mg/kg body weight, the therapeutically effective amount of MET is about 0.1 to about 200 mg/kg body weight, and the therapeutically effective amount of EGCG is about 0.5 mg/kg body weight to about 500 mg/kg body weight. In embodiments, the therapeutically effective amount of the chemotherapeutic agent is about 0.1 to about 200 mg/kg body weight, the therapeutically effective amount of MET is about 0.1 to about 200 mg/kg body weight, and the therapeutically effective amount of EGCG is about 0.5 mg/kg body weight to about 500 mg/kg body weight for a 70 kg human subject.

In embodiments, the chemotherapeutic agent, MET, and EGCG are administered concurrently. In embodiments, the chemotherapeutic agent, MET, and EGCG are administered sequentially. In embodiments, the MET and EGCG are administered concurrently and the chemotherapeutic agent is administered sequentially.

In embodiments, the chemotherapeutic agent, MET, and EGCG are administered via parenteral, intrathecal, subcutaneous, oral, nasal, inhalation, or sublingual administration.

In embodiments, the methods further comprise administering an additional anti-cancer therapy selected from radiation therapy, surgery, an additional chemotherapeutic agent, an anti-angiogenic agent, an immunotherapy, an oncolytic virus, and an anti-inflammatory agent.

In embodiments, the chemotherapeutic agent used in the methods of the invention is TMZ, sorafenib, cisplatin, the chemotherapeutic agent is TMZ, sorafenib, cisplatin, topotecan, pegylated liposomal doxorubicin (PLD), thiotepa, cyclosphosphamide, busulfan, improsulfan, piposulfan, aziridines, benzodopa, carboquone, meturedopa, uredopa, ethylenimines, altretamine, triethylenemelamine, trietylenephosphoramide, triethiylenethiophosphoramide, trimethylolomelamine, acetogenins, bullatacin, bullatacinone, a camptothecin, bryostatin; callystatin; CC-1065, adozelesin, carzelesin, bizelesin, cryptophycin 1, cryptophycin 8, dolastatin; duocarmycin, KW-2189, CB1-TM1, eleutherobin, pancratistatin, a sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, nitrosureas, carmustine, chlorozotocin, fotemustine, lomustine, nimustine, ranimnustine, enediyne antibiotics, calicheamicins, dynemicin A; bisphosphonates, clodronate, an esperamicin, neocarzinostatin, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin, deoxydoxorubicin, epirubicin, esorubicin, idarubicin, marcellomycin, mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; methotrexate, 5-fluorouracil (5-FU); folic acid analogues, denopterin, pteropterin, trimetrexate; purine analogs, fludarabine, 6-mercaptopurine, thiamiprine, thioguanine, pyrimidine analogs, ancitabine, azacytidine, 6-azauridine, carmofur, cytarabine, dideoxyuridine, doxifluridine, enocitabine, floxuridine; androgens, calusterone, dromostanolone propionate, epitiostanol, mepitiostane, testolactone; anti-adrenals, aminoglutethimide, mitotane, trilostane; folic acid replenishers, frolinic acid, aceglatone, aldophosphamide glycoside, aminolevulinic acid, eniluracil, amsacrine, bestrabucil, bisantrene, edatrexate, defofamine, demecolcine, diaziquone, elformithine, elliptinium acetate, an epothilone, etoglucid, gallium nitrate, hydroxyurea, lentinan, lonidainine, maytansinoids, maytansine, ansamitocins, mitoguazone, mitoxantrone, mopidanmol; nitraerine, pentostatin, phenamet, pirarubicin, losoxantrone, podophyllinic acid, 2-ethylhydrazide, procarbazine, razoxane, rhizoxin, sizofuran, spirogermanium, tenuazonic acid, triaziquone; 2,2′,2″-trichlorotriethylamine, trichothecenes, T-2 toxin, verracurin A, roridin A, anguidine, urethan, vindesine, dacarbazine, mannomustine, mitobronitol, mitolactol, pipobroman, gacytosine, arabinoside (“Ara-C”), cyclophosphamide, thiotepa, taxoids, paclitaxel, an albumin-engineered nanoparticle formulation of paclitaxel, docetaxel, chlorambucil, gemcitabine, 6-thioguanine, mercaptopurine, methotrexate, platinum analogs, oxaliplatin, carboplatin, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluoromethylornithine (DFMO), retinoic acid, capecitabine, combretastatin, leucovorin (LV), oxaliplatin, lapatinib, inhibitors of PKC-alpha, Raf, H-Ras, EGFR, erlotinib, VEGF inhibitors and pharmaceutically acceptable salts, acids or derivatives of any of the above.

In embodiments, the chemotherapeutic agent is selected from the group consisting of temozolomide, sorafenib, cisplatin, 5-fluorouracil and doxorubicin.

In embodiments, the chemotherapeutic agent is TMZ. In embodiments, the chemotherapeutic agent is sorafenib. In embodiments, the chemotherapeutic agent is cisplatin. In embodiments, the chemotherapeutic agent is 5-fluorouracil. In embodiments, the chemotherapeutic agent is doxorubicin.

### BRIEF DESCRIPTION OF THE DRAWINGS

FIGS. 1A-1H. Assessment of cell viability using MTT (3-(4,5-dimethylthiazolyl-2)-2,5-diphenyltetrazolium bromide). (1A) TMZ-treated glioma cells; (1B) MET-treated glioma cells; (1C) EGCG-treated glioma cells; (1D) TMZ+MET-treated glioma cells; (IE) TMZ+EGCG-treated glioma cells; (1F) MET+EGCG-treated glioma cells; (1G) TMZ+MET+EGCG-treated glioma cells; (1H) TMZ+EGCG+MET-treated glioma cells. Percentage viability is expressed as Mean±Standard Deviation with reference to the non-treated control. TMZ—Temozolomide, MET—metformin, EGCG—epigallocatechin gallate

FIG. 2. Viability of HEK293T cells as assessed by trypan blue exclusion. HEK293T cells were treated with IC₅₀ values of TMZ, MET, and EGCG, individually and in combinations determined on U87MG and C6 glioma cells. C—non treated cells, T—Temozolomide, M—metformin, E—epigallocatechin gallate.

FIG. 3. Combination Index (CI) for dual- and triple-drug combinations (T—Temozolomide, M—metformin, E—epigallocatechin gallate) in U87MG and C6 cells. A CI<1.0 represents a synergistic effect, a CI=1.0 is an additive effect, and a CI>1.0 represents an antagonistic drug interaction (**p<0.001).

FIGS. 4A-4H. Apoptotic cell death observed in U87MG human glioma cells as assessed by AO/EtBr staining on the indicated groups. (4A) Control group; (4B) T-treated glioma cells (early apoptotic cells); (4C) M-treated glioma cells (no apoptosis but loss of cell membrane integrity); (4D) E-treated glioma cells (no apoptosis); (4E) TM-treated glioma cells (late apoptotic cells); (4F) TE-treated glioma cells (early apoptosis and loss of cell membrane integrity); (4G) ME-treated glioma cells (no apoptosis but loss of cell mem-brane integrity); (4H) TME-treated glioma cells (necrotic cells with total loss of morphology); T—Temozolomide, M—metformin, E—epigallocatechin gallate.

FIGS. 5A-5H. Apoptotic cell death observed in C6 glioma cells as assessed by AO/EtBr staining on the indicated groups. (5A) Control group; (5B) T-treated glioma cells (early apoptotic cells); (5C) M-treated glioma cells (no apoptosis but loss of cell membrane integrity); (5D) E-treated glioma cells (no apoptosis); (5E) TM-treated glioma cells (late apoptotic cells); (5F) TE-treated glioma cells (early apoptosis and loss of cell membrane integrity); (5G) ME-treated glioma cells (no apoptosis but loss of cell membrane integrity); (5H) TME-treated glioma cells (necrotic cells with total loss of morphology); T—Temozolomide, M—metformin, E—epigallocatechin gallate.

FIGS. 6A-6B. Effect of TMZ, MET, and EGCG, individually and in combination, on the levels of (6A) intra-cellular reactive oxygen species (ROS) as measured using a DCFDA assay in U87MG and C6 cells; (6B) levels of thiobarbituric acid reactive substances (TBARS) in U87MG and C6 cells. The values are expressed as units of absorbance in optical density (OD) (***p<0.001, **p<0.01 compared with control); C—non treated GB cells, T—Temozolomide, M—metformin, E—epigallocatechin gallate.

FIGS. 7A-7D. Effect of T, M, and E, individually and in combination, on the levels of antioxidant enzymes in U87MG and C6 glioma cells. (7A) superoxide dismutase (SOD); (7B) catalase (CAT); (7C) glutathione peroxidase (GPx); (7D) reduced glutathione (GSH) (**p<0.01, *p<0.05 compared with control); C—non treated GB cells, T—Temozolomide, M—metformin, E—epigallocatechin gallate.

FIGS. 8A and 8B. Effect of T, M, and E, individually and in combination, on (8A) SOD and (8B) CAT mRNA expression in U87MG and C6 glioma cells. (**p<0.01, *p<0.05 compared with control); T—Temozolomide, M—metformin, E—epigallocatechin gallate.

FIGS. 9A-9B. Effect of T, M, and E, individually and in combination, on gene expression (9A) and protein (9B) levels of Nrf-2 in U87MG and C6 glioma cells respectively, as determined by quantitative RT-PCR and enzyme-linked immunosorbent assay, respectively. (**p<0.01, *p<0.05 compared with control); C—non treated GB cells, T—Temozolomide, M—metformin, E—epigallocatechin gallate.

FIG. 10. Effect of T, M, and E, individually and in combination on BCL2 mRNA expression in U87MG and C6 cells was determined by RT-PCR (**p<0.01 compared with control); T—Temozolomide, M—metformin, E—epigallocatechin gallate.

FIGS. 11A-11B. Effect of T, M, and E, individually and in combination, on gene expression (11A) and protein (11B) levels of caspase-9 in U87MG and C6 glioma cells, respectively, as determined by quantitative RT-PCR and enzyme-linked immunosorbent assay, respectively. (**p<0.01 compared with control)); C—non treated GB cells, T—Temozolomide, M—metformin, E—epigallocatechin gallate.

FIGS. 12A-12C. (12A) Table showing the treatment strategies followed in the present study (* Survival study; #Experimental studies). (12B) Kaplan-Meier survival curves were plotted and statistical analysis (Wilcoxon-Gehan test) was performed. (12C) Hematoxylin and eosin (H & E) staining of tumor sections from various treatment groups. Images were captured at 20× magnification, wherein the images 12C-a and 12C-b correspond to non tumor control (C) and Tumor contro (TC), while 12C-c to 12C-i correspond to treatment with temolozomide alone (T), metformin alone (M), epigallocatechin gallate alone (E), temozolomide plus metformin (TM), temozolomide plus epigallocatechin gallate (TE), metformin plus epigallocatechin gallate (ME) and combined temozolomide, metformin, and epigallocatechin gallate (TME), respectively.

FIGS. 13A and 13B. (13A) IHC staining for VEGF in tumor sections from different experimental groups 40× magnification. (13A-a to 13A-i) IHC staining for VEGF in various treatment groups, wherein cytoplasmic positivity and negativity are denoted by the black and red arrows respectively. (13A-j) Quantification of positivity for VEGF in each group. (13B) Ki67 nuclear expression in different experimental groups at 40× magnification. (13B-a to 13B-i) IHC staining for Ki67 in various treatment groups, wherein nuclear positivity and negativity are denoted by the black and red arrows respectively. (13B-j) Quantification of nuclear positivity for Ki67 in each group (**P<0.001, *** P<0.0001). Triple-drug combination significantly enhanced the ROS levels and levels of antioxidant and non-antioxidant enzymes in of glioma; T—Temozolomide, M—metformin, E—epigallocatechin gallate.

FIGS. 14A-14H. (14A) shows the cumulative ROS and TBARS activity observed in different treatment groups, in which the triple-drug treatment and the dual-drug treatment of TE significantly elevated both ROS and TBARS activity. (14B) Heat map for the gene expression levels of VEGF, VEGFR, PTEN, PI3K, PDK1, AKT1, mTOR, GSK30 PTEN, HIF-1α and Nrf2 the significance was calculated using two-way ANOVA. (14C) Western blot bands for markers of PI3K/AKT/GSK30/Nrf2 pathway (HIF-1α, Nrf2 PTEN, PI3K, pAKT1, PDK1 and pmTOR) analyzed for various treatment groups. (14D) Protein levels of VEGFR1 and VEGF by ELISA. (14E) Gene expression levels of apoptotic markers (BCL2 and caspase-9, -8 and -3). (14F) Western blot bands for apoptotic proteins (BAD & caspase-9) proteins analysed for various treatment groups. (14G) The intensities of apoptotic proteins BAD and caspase-9 band were calculated against β-Actin. (14H) Protein levels of caspase-8 & 3 were analyzed using ELISA. The significance was calculated using two-way ANOVA (**P<0.001, *** P<0.0001); C—non tumor control, TC—Tumor control, T—Temozolomide, M—metformin, E—epigallocatechin gallate.

FIGS. 15A-15F. (15A) Effect of TMZ, MET, and EGCG, individually and in combination on cell cycle, wherein the histograms represent the DNA content of the cells in each of the cell cycle phases (G0/G1, S and G2/M). Different cell cycle phases were plotted. (15B) Percentage of total events, wherein the bar-graph depicting the percentage of cells in G1, S and G2/M phases in each treatment group. (15C) Pie chart representing the percentage of cells in G1, S and G2/M phases in tumor-control and triple-drug treatment group respectively. (15D) Tabulation of percentage of cell in different phases in each treatment group. (15E) The clustered bars represent total percentages of live, apoptotic, and necrotic cells for the various treatment groups. (15F) Represent the cell population in the 4 different quadrants (Q1—early apoptotic, Q2—necrotic, Q3-late apoptotic, and Q4—live cells); C—non tumor control, TC—Tumor control, T—Temozolomide, M—metformin, E—epigallocatechin gallate.

FIGS. 16A and 16B. (16A) 3D structure of human VEGFR protein (3HNG) and 2D structures of the drugs employed in docking. (16B) Site maps of drug-protein reactions in the VEGFR active domains. The drugs are arranged in ascending order of their glide score where, (I) T, (II) MTIC, (III) AIC, (IV) M (V) GUA and (VI) E.

FIG. 17. The levels of antioxidant and non-antioxidant enzymes (SOD, GPx, CAT, GSH) wherein, the triple-drug combination significantly enhanced the levels of all the enzymes followed by the dual-drug treatment (TE) (*P<0.01, ** P<0.001); C—non tumor control, TC—Tumor control, T—Temozolomide, M—metformin, E—epigallocatechin gallate.

FIG. 18. Assessment of cell viability using MTT (3-(4,5-dimethylthiazolyl-2)-2,5-diphenyltetrazolium bromide) in human hepatocellular carcinoma (HepG2) cells treated with SOR, MET and EGCG individually and in triple-combination (SOR+MET+EGCG); SOR-Sorafenib, MET—metformin, EGCG—epigallocatechin gallate.

FIG. 19. Assessment of cell viability using MTT (3-(4,5-dimethylthiazolyl-2)-2,5-diphenyltetrazolium bromide) in human ovarian cancer (SKOV3) cells treated with CPL, MET and EGCG individually and in triple-combination (CPL+MET+EGCG); CPL-Cisplatin, MET—metformin, EGCG—epigallocatechin gallate.

FIG. 20. A) Assessment of cell viability using MTT (3-(4,5-dimethylthiazolyl-2)-2,5-diphenyltetrazolium bromide) in human pancreatic cancer (PANC1) cells treated with 5-Fluorouracil (5FU), metformin (MET) and epigallocatechin gallate (EGCG) individually and in triple-combination (5FU+MET+EGCG). B) Assessment of cell viability using MTT (3-(4,5-dimethylthiazolyl-2)-2,5-diphenyltetrazolium bromide) in human colon cancer (HCT116) cells treated with 5FU, MET and EGCG individually and in triple-combination (5FU+MET+EGCG).

FIG. 21. A) Assessment of cell viability using MTT (3-(4,5-dimethylthiazolyl-2)-2,5-diphenyltetrazolium bromide) in human breast cancer (MDA-MB-453) cells treated with Doxorubicin (DOX), metformin (MET) and epigallocatechin gallate (EGCG) individually and in triple-combination (DOX+MET+EGCG). B) Assessment of cell viability using MTT (3-(4,5-dimethylthiazolyl-2)-2,5-diphenyltetrazolium bromide) in human bone cancer (SaoS2) cells treated with DOX, MET and EGCG individually and in triple-combination (DOX+MET+EGCG).

FIG. 22. Glucose uptake by the GB cells and isolated astrocytes in various treatment groups. (A) U-87MG & C6 GB cells: C—non treated GB cells, T—Temozolomide, M—metformin, E—epigallocatechin gallate (B) astrocytes isolated from the tumor tissue: C—non tumor control, TC-Tumor control, T—Temozolomide, M—metformin, E—epigallocatechin gallate. Wherein the triple-drug combination most effectively reduced glucose uptake in both GB cells as well as GB xenograft model. All the experiments were performed in triplicates and each experiment was repeated thrice. The data are represented as mean±standard deviation and the significance was calculated using two-way ANOVA (*p<0.05, **p<0.01, ***<0.001).

FIG. 23. (A-B) Gene expression levels of GLUT1 & 4, PKM2, LDHV and MTC1 & 4: T—Temozolomide, M—metformin, E—epigallocatechin gallate. (A) U-87 MG Human GB cell line and (B) C6 rat GB cell line. (C) Rat brain tissue: TC—Tumor control, T—Temozolomide, M—metformin, E—epigallocatechin gallate. The triple-drug combination most significantly suppressed the gene expression of all the makers of Warburg effect followed by the dual treatment with TM. (D-E) Protein levels of GLUT1, PKM2, LDHV and MTC1; C—non treated GB cells, T—Temozolomide, M—metformin, E—epigallocatechin gallate. (D) U-87 MG Human GB cell line and (E) C6 rat GB cell line. (F) Rat brain tissue: C—non tumor control, TC—Tumor control, T—Temozolomide, M—metformin, E—epigallocatechin gallate. The triple-drug combination of TME followed by dual combination of TM significantly protein levels GLUT1, PKM2, LDHV and MCT1. All the experiments were performed in triplicates and each experiment was repeated thrice. The data are represented as mean±standard deviation and the significance was calculated using two-way ANOVA (**p<0.01, ***<0.001). (G) Spearman correlation between qRT-PCR and ELISA data (rho value of 0.69). Significant correlation between qPCR and ELISA and the two-tailed p<0.001.

FIG. 24. Histopathological analysis (H&E): H & E staining of tumor sections from Tumor control, Dose-1, Dose-2 and Dose-3 treatment groups. Images were captured at 4× and 45× magnification. The tumor control group showed whorled structures (red arrows) along with angiogenic sites (black arrow) with high cellular density. The Dose 1 treatment reduced angiogenesis however whorled tumor section was still persistent with high cellularity. The Dose 3 treatment effectively reduced cellularity and no whorled structures or angiogenesis was observed.

The results highlighted the of the three TME doses, Dose 3 has the most effective anti-glioma efficacy.

FIG. 25. Toxicity analysis in vital organs: H & E staining of vital organs from Dose-1, Dose-2 and Dose-3 treatment groups. Images were captured at 4× and 45× magnification. Neither of the treatment groups induced any significant toxicity.

FIG. 26. Overall survival (OS) by Kaplan-Meier plot: This data was displayed for the three distinct T—Temozolomide, M—metformin and E—epigallocatechin gallate doses with reference to tumour control (TC) animals, which shows that Dose-1 and Dose-2 increased average OS by 17 and 18 weeks, respectively, while Dose-3 was the most successful with an OS of more than 23 weeks. The median survival rate of the Dose-3 treated animals was significantly higher than the tumor-control group (>23 weeks vs. 5.6 weeks; Gehan-Breslow-Wilcoxon test; p<0.001).

### DETAILED DESCRIPTION OF THE INVENTION

The present disclosure relates to compositions comprising MET and EGCG. In embodiments, the compositions further comprise a chemotherapeutic agent. In embodiments, the compositions are pharmaceutical compositions or formulations comprising MET and EGCG, and optionally further comprising a chemotherapeutic agent, and optionally a pharmaceutically acceptable excipient, carrier, or diluent. In embodiments, the chemotherapeutic agent is TMZ. In embodiments, the chemotherapeutic agent is sorafenib. In embodiments, the chemotherapeutic agent is cisplatin.

The present disclosure also provides kits for inhibiting growth of cancer cells, promoting apoptosis of cancer cells, and/or treating or ameliorating cancer in a subject in need thereof, wherein the kits comprise a composition or compositions comprising amounts or doses of MET and EGCG that are effective for inhibiting growth or promoting apoptosis of a cancer cell, or treating or ameliorating cancer in a subject in need thereof. In embodiments, the kits further comprise a composition comprising amounts or doses of a chemotherapeutic agent that are effective for inhibiting growth or promoting apoptosis of a cancer cell, and/or treating or ameliorating cancer in a subject in need thereof. In embodiments, the chemotherapeutic agent is TMZ. In embodiments, the chemotherapeutic agent is sorafenib. In embodiments, the chemotherapeutic agent is cisplatin.

The present disclosure further relates to methods of inhibiting growth or promoting apoptosis of a cancer cell comprising contacting the cancer cell with an effective amount of a chemotherapeutic agent, an effective amount of MET, and an effective amount of EGCG. The present disclosure also relates to methods of treatment or amelioration of a cancer in a subject in need thereof, comprising a therapeutically effective amount of a chemotherapeutic agent, a therapeutically effective amount of MET, and a therapeutically effective amount of EGCG to the subject. In embodiments, the methods of the invention provide a synergistic inhibition of cancer cell growth or a synergistic promotion of cancer cell apoptosis, relative to administration of a chemotherapeutic agent, MET, and EGCG individually. In embodiments, the methods of the invention provide a synergistic treatment or amelioration of cancer relative to treatment or amelioration of the cancer provided by administering a chemotherapeutic agent, MET, and EGCG individually. In embodiments, the chemotherapeutic agent is TMZ.

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. As used herein, “a” or “an” may mean one or more. As used herein, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability, depending on the situation.

The use of the term “or” in the claims is used to mean “and/or”, unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps. It is contemplated that any embodiment discussed in this specification can be implemented with respect to any protein, compositions, polynucleotides, vectors, cells, methods, and/or kits of the present disclosure. Furthermore, compositions, polynucleotides, vectors, cells, and/or kits of the present disclosure can be used to achieve methods and proteins of the present disclosure.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

As used herein, “treatment” (and grammatical variations thereof such as “treat” or “treating”) refers to clinical intervention in an attempt to alter the natural course of the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, increasing/extending overall survival (OS), increasing/extending progression-free survival (PFS), decreasing the rate of disease progression, amelioration or palliation of the disease state, and remission or improved prognosis. The terms “ameliorate”, “ameliorating”, and “amelioration” encompass improving a subject's symptoms or overall condition.

The term “effective amount” refers to an amount of a drug or combination of drugs effective to inhibit cell growth, promote apoptosis, and/or treat a disease or disorder, such as glioblastoma, in a cell, or in a subject or patient, such as a mammal, e.g., a human. In the case of treatment of amelioration of a cancer, such as glioblastoma, the therapeutically effective amount of the drug or combination of drugs may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer (e.g., glioblastoma). To the extent the drug or combination of drugs may prevent growth and/or kill existing cancer cells, it may be cytostatic and/or cytotoxic and/or promote apoptosis. For cancer therapy, efficacy in vivo can, for example, be measured by assessing the duration of survival, duration of progression free survival (PFS), overall survival (OS), the response rates (RR), duration of response, and/or quality of life.

“Survival” refers to the subject remaining alive and includes progression-free survival (PFS) and overall survival (OS). Survival can be estimated by the Kaplan-Meier method, and any differences in survival are computed using the stratified log-rank test.

“Overall survival” or “OS” refers to the subject remaining alive for a defined period of time, such as about 1 year, about 2 years, about 3 years, about 4 years, about 5 years, about 10 years, etc., from initiation of treatment or from initial diagnosis.

“Overall response rate” or “Objective response rate” (ORR) refers to the percentage of people who experience a decrease in the size or amount of the cancer for a minimum amount of time, and ORR can be represented by the sum of the complete and partial response rates.

“Progression-free survival” or “PFS” refers to the time from treatment (or randomization) to first disease progression or death. For example, it is the time that the subject remains alive, without return of the cancer, e.g., for a defined period of time such as about 1 month, about 2 months, about 3 months, about 4, months, about 5 months, about 6 months, about 7 months, about 8 months, about 9 months, about 1 year, about 2 years, about 3 years, etc., from initiation of treatment or from initial diagnosis. In one aspect of the invention, PFS can be assessed by the MacDonald Response Criteria as described in MacDonald et al. (J. Clin. Oncol. 1990; 8: 1277-80, 1990).

A “patient” or “subject” herein refers to any single animal (including, for example, a mammal, such as a dog, a cat, a horse, a rabbit, a zoo animal, a cow, a pig, a sheep, a non-human primate, and a human), such as a human, eligible for treatment who is experiencing or has experienced one or more signs, symptoms, or other indicators of a disease or disorder. Intended to be included as a patient are any patients involved in clinical research trials not showing any clinical sign of disease, or patients involved in epidemiological studies, or patients once used as controls. The patient may have been previously treated with an any anti-cancer treatment, or not so treated. The patient may be naïve to an additional drug(s) being used when the treatment herein is started, i.e., the patient may not have been previously treated with a chemotherapeutic agent, MET, and/or EGCG at “baseline” (i.e., at a set point in time before the administration of a first dose of a chemotherapeutic agent, MET, and EGCG) in the treatment method herein, such as the day of screening the subject before treatment is commenced). Such “naïve” patients or subjects are generally considered to be candidates for treatment with such additional drug(s).

### Compositions

As used herein, a “pharmaceutical” composition is a composition or formulation which suitable for administration to an animal, including a mammal, such as a dog, a cat, a horse, a rabbit, a zoo animal, a cow, a pig, a sheep, a non-human primate, and a human. In embodiments, the pharmaceutical composition is suitable for administration to a human. In embodiments, the pharmaceutical composition further comprises one or more pharmaceutically acceptable excipients, diluents, or carriers.

The present disclosure provides pharmaceutical compositions comprising MET and EGCG. In embodiments, the pharmaceutical composition further comprises a chemotherapeutic agent. In embodiments, the chemotherapeutic agent in the pharmaceutical composition comprising a chemotherapeutic agent, MET, and EGCG is any chemical compound except metformin and epigallocatechin gallate.

In embodiments, the chemotherapeutic agent is selected from the group consisting of temozolomide, sorafenib, cisplatin, 5-fluorouracil and doxorubicin. In embodiments, the chemotherapeutic agent is temozolomide. In embodiments, the chemotherapeutic agent is sorafenib. In embodiments, the chemotherapeutic agent is cisplatin. In embodiments, the chemotherapeutic agent is 5-fluorouracil. In embodiments, the chemotherapeutic agent is doxorubicin. In embodiments, the chemotherapeutic agent is not metformin. In embodiments, the chemotherapeutic agent is not epigallocatechin gallate. In embodiments, the chemotherapeutic agent is not a combination of metformin and epigallocatechin gallate.

In embodiments, the chemotherapeutic agent is any chemical compound useful in the treatment of cancer. In embodiments, the chemotherapeutic agent is any chemical compound useful in the treatment of cancer except metformin and epigallocatechin gallate. Examples of chemotherapeutics agents include, but are not limited to, temozolomide, sorafenib, paclitaxel, topotecan, pegylated liposomal doxorubicin (PLD), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin; bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma 1 and calicheamicin omega 1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® docetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DFMO) retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above. In embodiments, the composition may comprise any combination of these or other chemotherapeutic agents. In a preferred embodiment, the chemotherapeutic agent is selected from the group consisting of temozolomide, sorafenib, cisplatin, 5-fluorouracil and doxorubicin, and is in particular temozolomide.

In embodiments, the pharmaceutical composition or formulation comprises MET and EGCG in amounts which are effective for inhibiting growth or promoting apoptosis of a cancer cell. In embodiments, the pharmaceutical composition further comprises a chemotherapeutic agent in an amount which is effective for inhibiting growth or promoting apoptosis of a cancer cell. In embodiments, the pharmaceutical composition or formulation comprises MET and EGCG in amounts which aretherapeutically effective for treatment or amelioration of a cancer when administered to a subject in need thereof. In embodiments, the pharmaceutical composition or formulation further comprises a chemotherapeutic agent in an amount which is therapeutically effective for treatment or amelioration of a cancer when administered to a subject in need thereof. In embodiments, the chemotherapeutic agent is TMZ. In embodiments, the chemotherapeutic agent is sorafenib. In embodiments, the chemotherapeutic agent is cisplatin. In embodiments, the chemotherapeutic agent is 5-fluorouracil. In embodiments, the chemotherapeutic agent is doxorubicin.

In embodiments, the pharmaceutical composition or formulation is suitable for administration by via parenteral, intrathecal, topical, oral, sublingual, subcutaneous, intraperitoneal, intrapulmonary, nasal, inhalation, and/or intralesional/intratumoral administration.

In embodiments, the compositions can be formulated as a liquid for parenteral, intrathecal, subcutaneous, intraperitoneal, or intralesional/intratumoral injection. In embodiments, the compositions can be in the form of a pill, capsule, gel, tablet, dragee, sachet, effervescent, lozenge, cream, or powder.

In embodiments, the chemotherapeutic agent, MET, and EGCG are present in the composition in an amount from 0.001 to 500 mg. In embodiments, the amount of chemotherapeutic agent, MET, and EGCG are present in the composition in an amount from 0.001 to 400 mg, about 0.001 to about 300 mg, about 0.001 to about 200 mg, about 0.001 to about 100 mg, about 0.001 to about 75 mg, about 0.001 to about 50 mg, about 0.001 to about 25 mg, about 0.001 to about 10 mg, about 0.001 to about 1 mg, about 0.001 to about 0.01 mg, or about 0.001 to about 0.1 mg. In embodiments, the amount of the chemotherapeutic agent, MET and EGCG are present in an amount of 0.01 to about 400 mg, about 0.1 to about 300 mg, about 1 to about 200 mg, about 10 to about 100 mg, about 25 to about 75 mg. In embodiments, the amount of chemotherapeutic agent, MET, and EGCG are present in the composition in an amount of about 0.001, 0.01, 0.1, 1, 10, 25, 50, 75, 100, 125, 150, 175, 200, 225, 250, 275, 300, 325, 350, 375, 400, 425, 450, 475, or 500 mg. In embodiments, the amounts of each of the chemotherapeutic agent, MET, or EGCG are the same. In embodiments, the amounts of each of the chemotherapeutic agent, MET, or EGCG are different. In embodiments, the amounts of each of the chemotherapeutic agent are in mg/kg for a 70 kg human subject.

Compositions and formulations of the agents used described herein, used in accordance with the present invention, are prepared by methods which are known in the art and comprise mixing a chemotherapeutic agent, MET, and EGCG with pharmaceutically acceptable carriers, excipients, diluents, or stabilizers. For general information concerning formulations, see, e.g., Gilman et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Eastori, Pa.; Avis et al., (eds.) (1993) Pharmaceutical Dosage Forms: Parenteral Medications Dekker, N.Y.; Lieberman et al., (eds.) (1990) Pharmaceutical Dosage Forms: Tablets Dekker, New York; and Lieberman et al., (eds.) (1990), Pharmaceutical Dosage Forms: Disperse Systems Dekker, New York, Kenneth A. Walters (ed.) (2002) Dermatological and Transdermal Formulations (Drugs and the Pharmaceutical Sciences), Vol 119, Marcel Dekker. In embodiments, the formulations to be used for in vivo administration are sterilized using methods known in the art, e.g. by filtration through sterile filtration membranes.

### Kits

The present disclosure provides a kit for inhibiting growth or promoting apoptosis of a cancer cell, or for treatment or amelioration of cancer in a subject in need thereof.

In embodiments, the kit comprises a pharmaceutical composition or formulation comprising MET and EGCG. In embodiments, the kit further comprises a pharmaceutical composition comprising a chemotherapeutic agent. In embodiments, the chemotherapeutic agent, MET, and EGCG are each present in amounts which are effective to inhibit growth or promote apoptosis in a cancer cell, and/or amounts which are therapeutically effective for treatment or amelioration of a cancer in a subject in need thereof. In embodiments, the chemotherapeutic agent is TMZ. In embodiments, the chemotherapeutic agent is sorafenib. In embodiments, the chemotherapeutic agent is cisplatin. In embodiments, the chemotherapeutic agent is not MET. In embodiments, the chemotherapeutic agent is not EGCG. In embodiments, the chemotherapeutic agent is not a combination of MET and EGCG.

In embodiments, the chemotherapeutic agent of the kit is any chemical compound useful in the treatment of cancer. In embodiments, the chemotherapeutic agent is any chemical compound useful in the treatment of cancer except metformin and epigallocatechin gallate. Examples of chemotherapeutics agents include, but are not limited to, temozolomide, sorafenib, paclitaxel, topotecan, pegylated liposomal doxorubicin (PLD), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin; bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® docetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DFMO) retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above. In embodiments, the composition of the kit may comprise any combination of these or other chemotherapeutic agents. In a preferred embodiment, the chemotherapeutic agent is selected from the group consisting of temozolomide, sorafenib, cisplatin, 5-fluorouracil and doxorubicin, and is in particular temozolomide.

In embodiments, the chemotherapeutic agent, MET, and EGCG are in the same composition or formulation. In embodiments, the chemotherapeutic agent, MET, and EGCG are in separate compositions or formulations. In embodiments, the chemotherapeutic agent is in a separate composition or formulation and the MET and EGCG are in the same composition or formulation.

In embodiments, the pharmaceutical compositions of the kit are suitable for administration by via parenteral, intrathecal, topical, oral, sublingual, subcutaneous, intraperitoneal, intrapulmonary, nasal, inhalation, and/or intralesional/intratumoral administration.

In embodiments, the compositions of the kit can be formulated as a liquid for parenteral, intrathecal, subcutaneous, intraperitoneal, or intralesional/intratumoral injection. In embodiments, the compositions can be in the form of a pill, capsule, gel, tablet, dragee, sachet, effervescent, lozenge, cream, or powder.

In embodiments, the chemotherapeutic agent, MET, and EGCG are present in the compositions of the kit in an amount from 0.001 to 500 mg. In embodiments, the amount of chemotherapeutic agent, MET, and EGCG are present in the composition in an amount from 0.001 to 400 mg, about 0.001 to about 300 mg, about 0.001 to about 200 mg, about 0.001 to about 100 mg, about 0.001 to about 75 mg, about 0.001 to about 50 mg, about 0.001 to about 25 mg, about 0.001 to about 10 mg, about 0.001 to about 1 mg, about 0.001 to about 0.01 mg, or about 0.001 to about 0.1 mg. In embodiments, the amount of the chemotherapeutic agent, MET and EGCG are present in an amount of 0.01 to about 400 mg, about 0.1 to about 300 mg, about 1 to about 200 mg, about 10 to about 100 mg, about 25 to about 75 mg. In embodiments, the amount of chemotherapeutic agent, MET, and EGCG are present in the composition in an amount of about 0.001, 0.01, 0.1, 1, 10, 25, 50, 75, 100, 125, 150, 175, 200, 225, 250, 275, 300, 325, 350, 375, 400, 425, 450, 475, or 500 mg. In embodiments, the amounts of each of the chemotherapeutic agent, MET, and EGCG are the same. In embodiments, the amounts of each of the chemotherapeutic agent, MET, and EGCG are different. In embodiments, the amounts of each of the chemotherapeutic agent are in mg/kg for a 70 kg human subject.

In embodiments, the kit comprises an article of manufacture comprising one or more containers, labels, and package inserts. Suitable containers include, for example, bottles, vials, syringes, etc. The containers may be formed from a variety of materials such as glass or plastic. The container holds a composition comprising a chemotherapeutic agent, MET, and EGCG which is effective for inhibiting cancer cell growth or promoting cancer cell apoptosis, or which is effective for treating or ameliorating cancer in a subject in need thereof. In embodiments, the kit comprises multiple containers, each of which holds a composition comprising a chemotherapeutic agent, MET, or EGCG. In embodiments, the one or more containers may have a sterile access port (for example the container may be an intravenous solution bag or a vial having a stopper pierceable by a hypodermic injection needle). The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, and syringes. In addition, the article of manufacture comprises a package insert with instructions for use, including for example instructing the user of the composition to administer the chemotherapeutic agent, MET, and EGCG composition to the patient/subject.

The chemotherapeutic agent, MET, and EGCG can be packaged alone or in combination with other anti-cancer therapeutic compounds as a kit. The kit can include optional components that aid in the administration of the unit dose to subjects, such as vials for reconstituting powder forms, syringes for injection, customized IV delivery systems, inhalers. In embodiments, the one or more compositions in the kit are formulated as tablets, pills, capsules, gels, sachets, effervescents, lozenges, creams, powders, etc. Additionally, the unit dose kit can contain instructions for preparation and administration of the compositions. The kit may be manufactured as a single use unit dose for one subject or multiple uses for a particular subject (at a constant dose or in which the individual compounds may vary in potency as therapy progresses). The kit may contain multiple doses suitable for administration to multiple subjects (“bulk packaging”). The kit components may be assembled in cartons, blister packs, bottles, tubes, and the like.

### Cancers and Cancer Cells

In embodiments, the cancer cell is a mammalian cancer cell. In embodiments, the mammalian cancer cell is a dog, cat, horse, rabbit, zoo animal, cow, pig, sheep, non-human primate, or a human cancer cell. In embodiments, the mammalian cancer cell is a human cancer cell.

In embodiments, the cancer to be treated or ameliorated is a cancer from a mammalian subject. In embodiments, the mammalian subject is a dog, a cat, a horse, a rabbit, a zoo animal, a cow, a pig, a sheep, a non-human primate, or a human. In embodiments, the subject is a human.

The terms “cancer” and “cancerous” refer to or describe the physiological condition in mammals that is typically characterized by unregulated cell growth. Included in this definition are benign and malignant cancers as well as dormant tumors or micrometastases.

In embodiments, the cancer cell or cancer to be treated or ameliorated includes, but is not limited to, carcinoma, lymphoma, blastoma, sarcoma, and leukemia. In embodiments, the cancer is a brain cancer, breast cancer, squamous cell cancer, lung cancer (including small-cell lung cancer, non-small cell lung cancer, adenocarcinoma of the lung, and squamous carcinoma of the lung), cancer of the peritoneum, liver cancer, including hepatocellular carcinoma, gastric or stomach cancer (including gastrointestinal cancer), pancreatic cancer, ovarian cancer, cervical cancer, liver cancer, bladder cancer, hepatoma, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. In embodiments, the cancer is selected from the group consisting of bone cancer, brain cancer, breast cancer, colon cancer, colorectal cancer, melanoma, pancreatic cancer, hepatocellular carcinoma, ovarian cancer, head and neck cancer, lung cancer, renal cancer, a lymphoma, a leukemia, a sarcoma, and a carcinoma. In a preferred embodiment, the cancer is bone cancer, brain cancer, breast cancer, colon cancer, pancreatic cancer, hepatocellular carcinoma, and ovarian cancer. In a more preferred embodiment, the cancer is brain cancer.

In embodiments, the brain cancer or brain cancer cell is an astrocytoma. In embodiments, the brain cancer or brain cancer cell is a glioma. In embodiments, the glioma or glioma cell is glioblastoma multiforme (GBM). In embodiments, the GBM is proneural GBM, neural GBM, classical GBM, and mesenchymal GBM. GBMs may be newly diagnosed, diagnosed, or recurrent. In embodiments, the cancer is a pancreatic cancer. In embodiments, the cancer is an ovarian cancer. In embodiments, the cancer is hepatocellular carcinoma. In embodiments, the cancer cell is a colon cancer cell. In embodiments, the cancer cell is a breast cancer cell. In embodiments, the cancer cell is a bone cancer cell.

### Chemotherapeutic Agents

In embodiments, the chemotherapeutic agent is any chemical compound useful in the treatment of cancer. In embodiments, the chemotherapeutic agent is any chemical compound useful in the treatment of cancer except metformin and epigallocatechin gallate. Examples of chemotherapeutics agents include, but are not limited to, temozolomide, paclitaxel, topotecan, pegylated liposomal doxorubicin (PLD), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin; bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® docetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DFMO) retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above. In embodiments, any combination of these chemotherapeutic agents is administered. In a preferred embodiment, the chemotherapeutic agent is selected from the group consisting of temozolomide, sorafenib, cisplatin, 5-fluorouracil and doxorubicin, and is in particular temozolomide.

In embodiments, the chemotherapeutic agent is temozolomide. In embodiments, the chemotherapeutic agent is sorafenib. In embodiments, the chemotherapeutic agent is cisplatin. In embodiments, the chemotherapeutic agent is 5-fluorouracil. In embodiments, the chemotherapeutic agent is doxorubicin. In embodiments, the chemotherapeutic agent is not metformin. In embodiments, the chemotherapeutic agent is not epigallocatechin gallate. In embodiments, the chemotherapeutic agent is not a combination of metformin and epigallocatechin gallate.

### Methods of Inhibiting Cell Growth or Promotion of Apoptosis

The present invention provides methods of inhibiting growth or of promoting apoptosis in a cancer cell. In embodiments, the cancer cell any of the aforementioned cancer cell types and is contacted with an effective amount of a chemotherapeutic agent, MET, and EGCG in vitro. In embodiments, the cancer cell is contacted with an effective amount of a chemotherapeutic agent, MET, and EGCG in vivo. In embodiments, the cancer cell is contacted with an effective amount of a chemotherapeutic agent, MET, and EGCG ex vivo. In embodiments, the chemotherapeutic agent is TMZ. In embodiments, the chemotherapeutic agent is sorafenib. In embodiments, the chemotherapeutic agent is cisplatin.

In embodiments, the effective amount of the chemotherapeutic agent is about 0.0001 mg to about 500 mg. In embodiment, the effective amount of the chemotherapeutic agent is about 0.001 mg to about 500 mg, about 0.01 mg to about 500 mg, about 0.1 mg to about 500 mg, about 1 mg to about 500 mg, In embodiments, the chemotherapeutic agent is TMZ. In embodiments, the effective amount of the chemotherapeutic agent is about 0.0001 mg/kg to about 500 mg/kg for a 70 kg human subject. In embodiment, the effective amount of the chemotherapeutic agent is about 0.001 mg/kg to about 500 mg/kg, about 0.01 mg to about 500 mg/kg, about 0.1 mg/kg to about 500 mg/kg, about 1 mg/kg to about 500 mg/kg for a 70 kg human subject. In embodiments, the chemotherapeutic agent is TMZ.

In embodiments, the effective amount of MET is about 0.0001 mg to about 300 mg. In embodiment, the effective amount of MET is about 0.001 mg to about 300 mg, about 0.01 mg to about 300 mg, about 0.1 mg to about 300 mg, about 1 mg to about 300 mg. In embodiments, the effective amount of MET is about 0.0001 mg/kg to about 300 mg/kg for a 70 kg human subject. In embodiment, the effective amount of MET is about 0.001 mg/kg to about 300 mg/kg, about 0.01 mg/kg to about 300 mg/kg, about 0.1 mg to about 300 mg/kg, about 1 mg to about 300 mg/kg for a 70 kg human subject.

In embodiments, the effective amount of EGCG is about 0.0001 mg to about 500 mg. In embodiment, the effective amount of EGCG is about 0.001 mg to about 500 mg, about 0.01 mg to about 500 mg, about 0.1 mg to about 500 mg, about 1 mg to about 500 mg. In embodiments, the effective amount of EGCG is about 0.0001 mg/kg to about 500 mg/kg for a 70 kg human subject. In embodiment, the effective amount of EGCG is about 0.001 mg/kg to about 500 mg/kg, about 0.01 mg/kg to about 500 mg/kg, about 0.1 mg/kg to about 500 mg/kg, about 1 mg/kg to about 500 mg/kg for a 70 kg human subject.

In embodiments, the cancer cell is contacted with a single dose of a chemotherapeutic agent, MET, and EGCG. In embodiments, the cancer cell is contacted with 2, 3, 4, 5, 6, 7, 8, 9, or 10 or more doses of a chemotherapeutic agent, MET, and EGCG.

In embodiments, the cancer cell is contacted with a chemotherapeutic agent, MET, and EGCG once per day. In embodiments, the cancer cell is contacted with a chemotherapeutic agent, MET, and EGCG once per day, every two days, every 3 days, once per week, once every two weeks, once every three week, or once per month.

In embodiments, the cancer cell is contacted with a chemotherapeutic agent, MET, and EGCG concurrently (i.e. at substantially the same time). In embodiments, the cancer cell is contacted with a chemotherapeutic agent, MET, and EGCG sequentially (i.e. at separate time points). In embodiments, the chemotherapeutic agent, MET, and EGCG are in a single composition or formulation. In embodiments, the chemotherapeutic agent, MET, and EGCG are in separate compositions or formulations. In embodiments, the MET and EGCG are in the same composition and the chemotherapeutic agent is in a separate composition.

In embodiments, the cancer is contacted with or exposed to an additional therapy or treatment. In embodiments, the additional therapy or treatment is ionizing radiation or radiotherapy, a chemotherapeutic agent, an anti-angiogenic agent, an immunotherapeutic agent, an oncolytic virus, or an anti-inflammatory agent.

### Methods of Treatment or Amelioration

The present invention provides methods of treating or ameliorating a cancer in a subject in need thereof. In embodiments, the cancer is any of the aforementioned cancer types.

In embodiments, the chemotherapeutic agent, MET, and EGCG are administered to the subject concurrently. In embodiments, the chemotherapeutic agent, MET, and EGCG are administered in the form of a single composition or formulation. In embodiments, the chemotherapeutic agent, MET, and EGCG are administered in separate compositions or formulations. In embodiments, the chemotherapeutic agent, MET, and EGCG are administered to the subject sequentially. In embodiments, the MET and EGCG are administered in the same composition, and the chemotherapeutic agent is administered in a separate composition. In embodiments, the chemotherapeutic agent is TMZ. In embodiments, the chemotherapeutic agent is sorafenib. In embodiments, the chemotherapeutic agent is cisplatin.

In embodiments, the therapeutically effective amount of the chemotherapeutic agent is about 0.1 to about 200 mg/kg body weight. In embodiments, the therapeutically effective amount of the chemotherapeutic agent is about 0.1 to about 175 mg/kg body weight, about 0.1 to about 150 mg/kg body weight, about 0.1 to about 125 mg/kg body weight, about 0.1 to about 100 mg/kg body weight. In embodiments, the therapeutically effective amount of the chemotherapeutic agent is about 0.1 to about 200 mg/kg body weight. In embodiments, the therapeutically effective amount of the chemotherapeutic agent is about 0.1 to about 175 mg/kg body weight for a 70 kg human subject, about 0.1 to about 150 mg/kg body weight, about 0.1 to about 125 mg/kg body weight, about 0.1 to about 100 mg/kg body weight for a 70 kg human subject. In embodiments, the chemotherapeutic agent is TMZ. In embodiments, the chemotherapeutic agent is sorafenib. In embodiments, the chemotherapeutic agent is cisplatin.

In embodiments, the therapeutically effective amount of MET is about 0.1 to about 200 mg/kg body weight. In embodiments, the therapeutically effective amount of MET is about 0.1 to about 175 mg/kg body weight, about 0.1 to about 150 mg/kg body weight, about 0.1 to about 125 mg/kg body weight, about 0.1 to about 100 mg/kg body weight. In embodiments, the therapeutically effective amount of MET is about 0.1 to about 200 mg/kg body weight for a 70 kg human subject. In embodiments, the therapeutically effective amount of MET is about 0.1 to about 175 mg/kg body weight, about 0.1 to about 150 mg/kg body weight, about 0.1 to about 125 mg/kg body weight, about 0.1 to about 100 mg/kg body weight for a 70 kg human subject.

In embodiments, the therapeutically effective amount of EGCG is about 0.5 mg/kg body weight to about 500 mg/kg body weight. In embodiments, the therapeutically effective amount of EGCG is about 1 to about 500 mg/kg body weight, about 5 to about 500 mg/kg body weight, about 10 to about 500 mg/kg body weight, about 25 to about 500 mg/kg body weight, about 50 to about 500 mg/kg body weight, about 75 to about 500 mg/kg body weight, about 100 to about 500 mg/kg body weight. In embodiments body weight is 70 kg and subject is human.

In embodiments, the amount of chemotherapeutic agent, MET, and EGCG can be expressed in terms of mg/m² body area. In embodiments, chemotherapeutic agent, MET, and EGCG are each administered to a subject at a dose of about 10 mg/m² to about 100 mg/m². In embodiments, the chemotherapeutic agent is TMZ. In embodiments, the chemotherapeutic agent is sorafenib. In embodiments, the chemotherapeutic agent is cisplatin.

In embodiments, the chemotherapeutic agent, MET, and EGCG are administered to the subject via parenteral, intrathecal, topical, oral, sublingual, subcutaneous, intraperitoneal, intrapulmonary, nasal, inhalation, and/or intralesional/intratumoral administration.

In embodiments, the chemotherapeutic agent, MET, and EGCG are administered via the same route of administration. In embodiments, the chemotherapeutic agent, MET, and EGCG are administered via different routes of administration.

In embodiments, the chemotherapeutic agent, MET, and EGCG are administered one time. In embodiments, the chemotherapeutic agent, MET, and EGCG are administered 2, 3, 4, 5, 6, 7, 8, 9, or 10 or more times. In embodiments, the chemotherapeutic agent, MET, and EGCG are administered once per day. In embodiments, the chemotherapeutic agent, MET, and EGCG are administered every day, every 2 days, every 3 days, every 4 days, every 5 days, every week, every two weeks, every three weeks, monthly, every two months, or more.

In embodiments, the chemotherapeutic agent, MET, and EGCG are administered in combination with one or more additional anti-cancer therapies. In embodiments, the additional anti-cancer therapy is ionizing radiation or radiotherapy, surgery, an additional chemotherapeutic agent, an anti-angiogenic agent, an immunotherapy, an oncolytic virus, and an ant-inflammatory agent. In embodiments, the one or more additional anti-cancer therapy is administered concurrently with the chemotherapeutic agent, MET, and EGCG. In embodiments, the one or more additional anti-cancer therapy is administered sequentially with the chemotherapeutic agent, MET, and EGCG.

In embodiments, therapy with ionizing radiation or radiotherapy is concurrent with the chemotherapeutic agent, MET, and EGCG administration. In embodiments, treatment with the chemotherapeutic agent, MET, and EGCG follows therapy with ionizing radiation or radiotherapy.

In embodiments, treatment with the chemotherapeutic agent, MET, and EGCG is prior to therapy with ionizing radiation or radiotherapy. In embodiments, the subject is treated with one cycle of ionizing radiation or radiotherapy and a chemotherapeutic agent, MET, and EGGC. In embodiments, the subject is treated with 2, 3, 4, 5, 6, 7, 8, 9, or 10 or more cycles of ionizing radiation or radiotherapy and a chemotherapeutic agent, MET, and EGCG.

In embodiments, the total dose of radiation administered to a subject is up to 100 gray (Gy). In some cases, the total dose of radiation administered to a subject is up to 20 Gy, 30 Gy, 35 Gy, 40 Gy, 45 Gy, 50 Gy, 55 Gy, 60 Gy, 65 Gy, 70 Gy, 75 Gy, 80 Gy, 85 Gy, 90 Gy, 95 Gy, or 100 Gy. In some cases, the total dose of radiation administered to a subject is up to 20 Gy. In some cases, the total dose of radiation administered to a subject is up to 30 Gy. In some cases, the total dose of radiation administered to a subject is up to 35 Gy. In some cases, the total dose of radiation administered to a subject is up to 40 Gy. In some cases, the total dose of radiation administered to a subject is up to 45 Gy. In some cases, the total dose of radiation administered to a subject is up to 50 Gy. In some cases, the total dose of radiation administered to a subject is up to 55 Gy. In some cases, the total dose of radiation administered to a subject is up to 60 Gy. In some cases, the total amount of radiation administered to the subject is up to 65 Gy. In some cases, the total amount of radiation administered to the subject is up to 70 Gy. In some cases, the total amount of radiation administered to the subject is up to 75 Gy. In some cases, the total amount of radiation administered to the subject is up to 80 Gy. In some cases, the total amount of radiation administered to the subject is up to 85 Gy. In some cases, the total amount of radiation administered to the subject is up to 90 Gy. In some cases, the total amount of radiation administered to the subject is up to 95 Gy. In some cases, the total amount of radiation administered to the subject is up to 100 Gy.

In embodiments, an additional chemotherapeutic agent is administered concurrently with the chemotherapeutic agent, MET, and EGCG. In embodiments, the additional chemotherapeutic agent is administered sequentially with the TMZ, sorafenib, or cisplatin, and the MET and EGCG.

In embodiments, the additional chemotherapeutic agent includes, but is not limited to, paclitaxel, topotecan, pegylated liposomal doxorubicin (PLD), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin; bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® docetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DFMO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above. In embodiments, any combination of these chemotherapeutic agents is administered. In a preferred embodiment, the chemotherapeutic agent is selected from the group consisting of temozolomide, sorafenib, cisplatin, 5-fluorouracil and doxorubicin, and is in particular temozolomide.

In embodiments, the anti-angiogenic agent is a small molecular weight substance, a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. The anti-angiogeneic agent includes those agents that bind and block the angiogenic activity of the angiogenic factor or its receptor. For example, an anti-angiogenesis agent is an antibody or other antagonist to an angiogenic agent as defined throughout the specification or known in the art, e.g., but are not limited to, antibodies to VEGF-A or to the VEGF-A receptor (e.g., KDR receptor or Flt-1 receptor), VEGF-trap, anti-PDGFR inhibitors such as Gleevec™ (Imatinib Mesylate). Anti-angiogensis agents also include native angiogenesis inhibitors, e.g., angiostatin, endostatin, etc. See, e.g., Klagsbrun and D'Amore, Annu. Rev. Physiol., 53:217-39 (1991); Streit and Detmar, Oncogene, 22:3172-3179 (2003) (e.g., Table 3 listing anti-angiogenic therapy in malignant melanoma); Ferrara & Alitalo, Nature Medicine 5:1359-1364 (1999); Tonini et al., Oncogene, 22:6549-6556 (2003) (e.g., Table 2 listing known antiangiogenic factors); and Sato. Int. J. Clin. Oncol., 8:200-206 (2003) (e.g., Table 1 lists anti-angiogenic agents used in clinical trials). In embodiments, the anti-angiogenic agent is a VEGF antagonist that inhibits VEGF/VEGF-R. In embodiments, the VEGF inhibitor is an anti-VEG antibody. In embodiments, the anti-VEGF antibody is bevacizumab (Avastin®), ramucirumab, or ranibizumab. In embodiments, the VEGF antagonist is a small molecule inhibitor. In embodiments, the VEGF small molecule inhibitor is axitinib, cabozanitinib, lapatinib, Lenvatinib, pazopanib, ponatinib, regorafenib, sorafenib, sunitinib, or vandetanib, or combinations thereof.

In embodiments, the additional therapeutic agent is an inhibitor of poly ADP ribose polymerase (PARP). In embodiments, the PARP inhibitor is Olaparib, Rucaparib, Niraparib, Talazoparib, Veliparib, CEP 9722, E7016, Iniparib, 3-aminobenzamide, or any combination thereof.

In embodiments, the additional therapeutic agent is an inhibitor of the Kirsten rat sarcoma virus (KRAS) protein. In embodiments, the KRAS inhibitor targets KRAS with a G12C mutation. In embodiments, the KRAS inhibitor is sotorasib.

In embodiments, the immunotherapy can be a cancer vaccine specific for a tumor-associated antigen, administration of a cytokine, interferon, interleukin, an anti-cytokine antibody, an anti-interferon antibody, an anti-interleukin antibody, a therapeutic antibody such as anti-PSCA or anti-HER2, anti-HER3, anti-HER4, or an adoptive T cell therapy such as CAR-T therapy, or combinations thereof, or an immune check point inhibitor.

In embodiments, the cytokine is interleukin (IL)-2, IL-12, IL-15, IL-18, IL-21, IL-23, IL-27, or combinations thereof. In embodiments, the interferon is a type I interferon, a type II interferon, a type III interferon, or combinations thereof. In embodiments, the type I interferon is an interferon-α, an interferon-β, an interferon-ε, an interferon-κ, an interferon-ω, or combinations thereof. In embodiments, the type II interferon is interferon-γ. In embodiments, the type III interferon is an interferon-λ1, interferon-λ1, interferon-λ3 (also known in the art as IL-29, IL-28A, and IL-28B, respectively). In embodiments, the type III interferon is interferon-λ4.

In embodiments, the therapeutic antibody targets programmed death ligand-1 (PD-L1). In embodiments the antibody that targets PD-L1 is atezolizumab, avelumab, durvalumab, or combinations thereof. In embodiments, the therapeutic antibody targets programmed cell death protein-1 (PD-1). In embodiments, the antibody that targets PD-1 is nivolumab, pembrolizumab, or combinations thereof. In embodiments, the therapeutic antibody targets CD20. In embodiments, the antibody that targets CD20 is ofatumumab, rituximab, or combinations thereof. In embodiments, the therapeutic antibody targets CD52. In embodiments, the antibody that targets CD52 is alemtuzumab. In embodiments, the therapeutic antibody targets CTLA-4. In embodiments, the antibody that targets CTLA-4 is ipilimumab. In embodiments, the therapeutic antibody targets SLAMF7. In embodiments, the antibody that targets SLAMF7 is elotuzumab. In embodiments, the therapeutic antibody targets CD47, G2D, PSCA, HER2, HER3, or HER4. In embodiments, any combination of the above therapeutic antibodies can be administered.

In embodiments, the oncolytic virus is a virus that kills cancer cells without harm to healthy cells. In embodiments, the oncolytic virus is selected from a poliovirus, a herpes simplex virus, an adenovirus, a measles virus, a Zika virus, and a parvovirus. (see Low et. el., U.S. Pat. No. 10,610,583). In embodiments, the oncolytic virus encodes an anti-cancer therapeutic protein. In embodiments, the oncolytic virus is administered directly to the cancer via intratumoral administration.

In embodiments, the anti-inflammatory agent is a steroid, a non-steroidal anti-inflammatory agent (NSAID), a TNF inhibitor, an interleukin-1 inhibitor, a cyclooxygenase (COX) inhibitor, or combinations thereof.

All references cited herein, including patents, patent applications, papers, textbooks, and the like, and the references cited therein, to the extent that they are not already, are hereby incorporated herein by reference in their entirety.

### EXAMPLES

### Example 1—Effect of TMZ, MF, and EGCG on Glioma Cell Growth In Vitro

Cell lines and culture conditions. A human glioma cell line, U87MG, a rat glioma cell line, C6, and normal HEK293T (Human Embryonic Kidney) cells were obtained from National Centre for Cell Science (NCCS), Pune, India. The cells were sub-cultured in Dulbecco's Modified Eagle's Medium (DMEM) supplemented with 10% FBS at 5% CO₂ and 37° C. At 85% confluence, the cells were harvested using 0.25% trypsin and seeded in 25 cm² flasks, 96 well, and 6-well plates, according to the experiment being performed. The cells were allowed to attach 70% to the surface prior to treatment. A stock solution of all the drugs (10 mg/ml) was made in a vehicle and diluted to the required concentrations. Suspensions were aspirated 10 times before treatment. Cells treated with vehicle control were used as a control.

Cell viability assay. U87MG and C6 cells (5×10³ cells/ml) were seeded in 96-well plates and exposed to different individual-drug treated groups with varying concentrations (0, 10, 40, 80, 120, and 160 μM) for a period of 24 hours, whereas HEK293T cells (5×10³ cells/ml) were seeded at the inhibitory concentration (IC) at 50% of drugs, either alone or in combination. The individual-treated groups include Group I: untreated control glioma cells; Group II: TMZ-treated glioma cells; Group III: MET-treated glioma cells; and Group IV: EGCG-treated glioma cells.

For dual-drug treatments, the IC₂₅ value of TMZ was kept constant and combined with varying concentrations (0, 10, 40, 80, 120, and 160 μM) of MET (TMZ+MET, Group V) and EGCG (TMZ+EGCG, Group VI). Furthermore, the IC₂₅ value of MET was kept constant, and varying concentrations of EGCG (0, 10, 30, 60, and 90 M) were utilized (MET+EGCG Group VII).

For the triple-drug combination, the IC₂₅ value of TMZ+MET was kept constant and varying concentrations (0, 10, 40, 80, 120, and 160 μM) of EGCG (TMZ+MET+EGCG, Group VIII) were utilized. Similarly, the IC₂₅ value of TMZ+EGCG was kept constant and varying concentrations of MET (TMZ+EGCG+MET, Group IX) were utilized. After a 24 hour treatment period, the cells were allowed to react with MTT for 4 hours in dark at 37° C. At the end of the incubation period, the dark purple formazan crystals formed were solubilized with DMSO and the absorbance was measured spectrophotometrically at 595 nm (Molecular Devices Spectra-Max M5, USA). The experiments were repeated at least three times. To determine the cell viability, the percentage of viability was calculated as follows:

% viability=[(Optical density {OD} of treated cells−OD of Blank)/(OD of control−OD of blank)]×100.

Calculation of IC₅₀ value. The IC50 of a drug was determined using y=m(x)+c equation (where: ‘y’ & ‘x’ are the respective coordinates of y & x-axis, ‘m’ is the slope and ‘c’ is the constant) derived from the trendlines of each scattered plots MMT assay. Furthermore, to calculate the IC₅₀ of dual-drug combinations, an MTT assay was performed, wherein the more potent drug was used at a constant concentration of IC₂₅ (derived from the individual treatment), while the second, less potent drug was used at the varying concentrations (0, 10, 40, 80, 120 and 160 μM). The IC₅₀ and IC₂₅ were also calculated for the dual combinations. The IC₅₀ for the triple combination was calculated by keeping the IC₂₅ of the dual-combination constant, and the third drug was used in varying concentrations (0, 10, 40, 80, 120, and 160 μM) (Table 1).

Cytotoxicity on a normal cell line. HEK293T cells were seeded in 96 well plates (5×10⁵ cells/well), and the cells were treated with the IC₅₀ values of individual, dual, and triple-drug combinations which were previously determined in glioma cells (C6 and U87MG) in order to evaluate the cytotoxicity at 24 hours using a trypan blue exclusion assay.

Combination index of drugs. According to the Chou-Talalay method [20], The statistical Combination Index (CI) of the drugs was performed according to the Chou-Talay method (Ma et al, Stem Cell Reports, 2017 9(6):1948-1960) to determine whether various drug combinations were synergistic, additive, or antagonistic, as shown in Table 1. This was performed for Groups V-IX using the following formula:

Combination index (CI) of Drugs 1 & 2=(IC₅₀ of drug-1 in combination/IC₅₀ of drug-1 individually)+(IC₅₀ of drug-2 in combination/IC₅₀ of drug-2 individually).

TABLE 1
Criteria for drug interaction.

| CI Value | Outcome |
|---|---|
| <1 | Synergistic |
| =1 | Additive |
| >1 | Antagonistic |

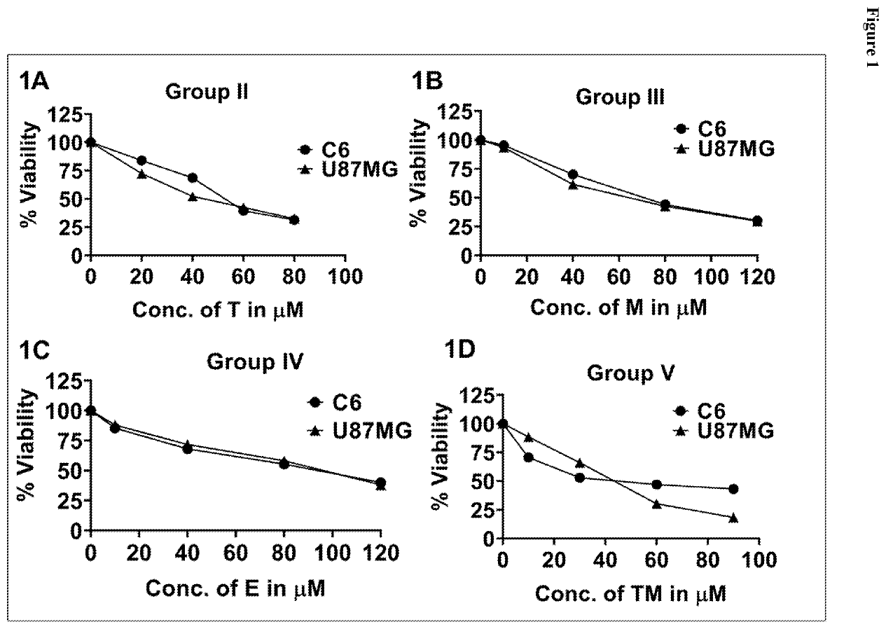

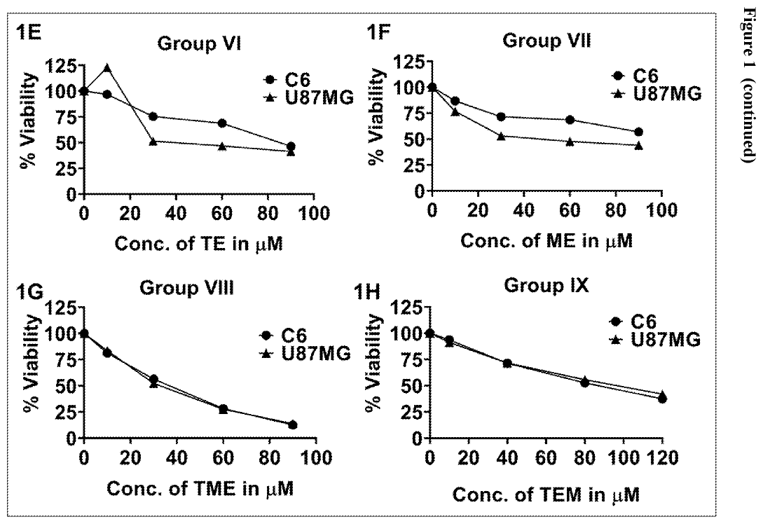

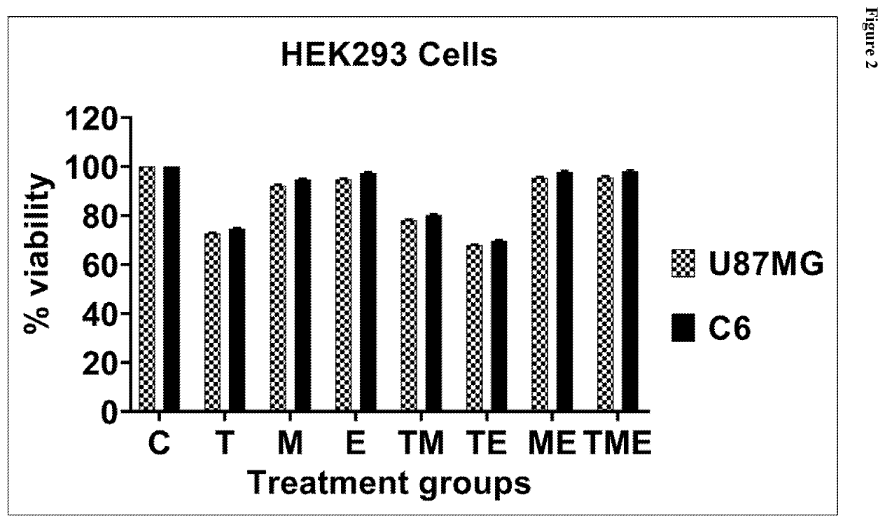

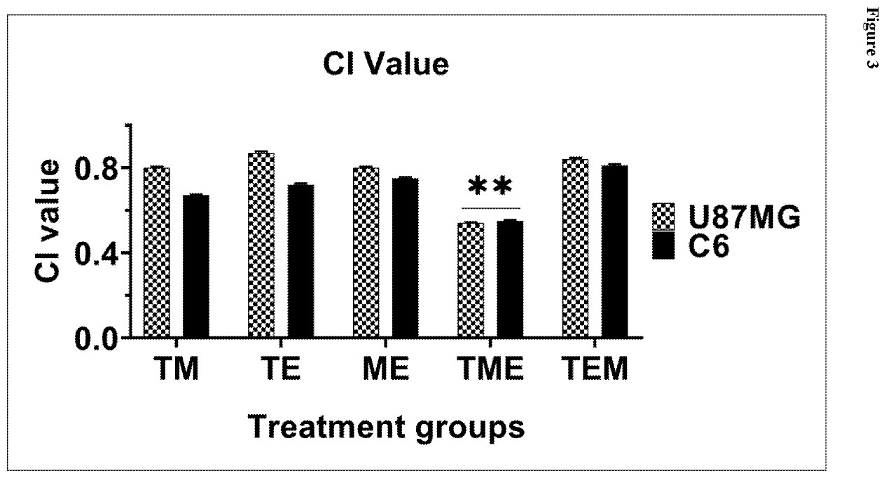

Results. The effect of TMZ, MET, and EGCG, alone and in combination, on glioma cell line growth was examined according to the methods described above. As shown in (FIG. 1), exposure of glioma cells to TMZ, MET, and EGCG resulted in a reduction in cell viability in a dose-dependent fashion. As depicted in FIG. 1G, the most significant anti-proliferative effect was observed in the triple-drug combination (TMZ+MET+EGCG, Group VIII), followed by the dual-drug combination (TMZ+MET, Group V) (FIG. 1D), and individually TMZ-treated (Group II) (FIG. 1A) and MET-treated (Group III) (FIG. 1B) glioma cells when compared to other treatment groups (p<0.05). TMZ+EGCG at their respective IC₂₅ values, when treated with varying concentrations of MET did not show any significant toxicity i.e., TMZ+EGCG+MET, Group IX (FIG. 1H). Because there was no significant change observed in the IC₅₀ value for both the cell lines, Group IX was excluded from further experiments. However, treatment with ECGC individually (Group IV) and in combination with TMZ (TMZ+EGCG, Group VI) or MET (EGCG+MET, Group VII) did not result in significant inhibitory effects (FIGS. 1E and 1F).

Furthermore, to check for the cytotoxicity of the drugs, a trypan blue dye exclusion assay was performed on a normal cell line, HEK293T, using the IC50 values of each drug, individually and in combination, as determined in U87MG and C6 cells and shown in Table 2.

TABLE 2
IC50 values of TMZ, MET, and EGCG, individually and in combination,
on GBM cell lines U87MG (human) and C6 (rat).

| Experimental | | Inhibitory Concentration at 50% (IC₅₀) | |
|---|---|---|---|
| Groups | Treatment | U87MG Cells | C6 Cells |
| Group I | Vehicle Control | NA | NA |
| | (DMSO) | | |
| Group II | TMZ | 77 ± 1.14 | 72 ± 1.32 |
| Group III | MET | 80 ± 1.19 | 77 ± 1.42 |
| Group IV | EGCG | 94 ± 1.39 | 96 ± 1.76 |
| Group V | TMZ + MET | (32 ± 0.48) + (63 ± 0.71) | (19 ± 0.36) + (51 ± 1.08) |
| Group VI | TMZ + EGCG | (32 ± 0.48) + (87 ± 0.89) | (19 ± 0.36) + (68 ± 1.44) |
| Group VII | MET + EGCG | (39 ± 0.58) + (72 ± 0.81) | (35 ± 0.64) + (67 ± 1.40) |
| Group VIII | TMZ + MET + | (32 ± 0.48) + (15 ± 0.17) + | (19 ± 0.36) + (24 ± 0.50) + |
| | EGCG | (43 ± 1.24) | (43 ± 1.09) |
| Group IX | TMZ + EGCG + | (32 ± 0.48) + (43 ± 0.48) + | (19 ± 0.36) + (24 ± 0.50) + |
| | MET | (83 ± 0.71) | (80 ± 0.68) |

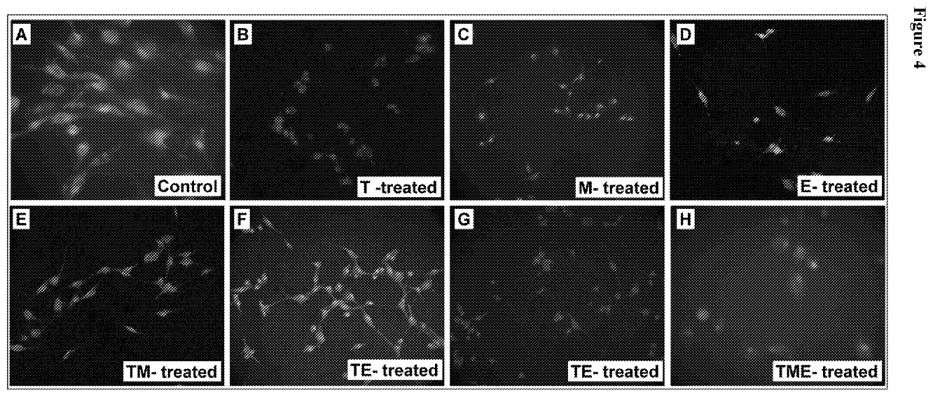

A significant reduction in toxicity was observed in normal cells treated with the triple-drug combination (TMZ+MET+EGCG), followed by dual-drug (MET+EGCG) and individual (MET and EGCG) drugs when compared with that of other treatment groups (FIG. 2). Interestingly, the TMZ-treated HEK293T cells exhibited higher toxicity, as depicted in (FIG. 2), yet the dual-drug (TMZ+MET)-treated HEK293T cells showed reduced toxicity; however, the change in toxicity levels was not significant. Based on the observations, the IC₅₀ concentrations of TMZ, MET, and EGCG in individual, dual, and triple combinations (Table 2) were selected further studies.

Based on the IC₅₀ values shown in Table 2, the Chou-Talalay method was used to calculate the combination index (CI). A CI below 1 is an indication of synergism (Table 1). CI values for the triple combination of TMZ (IC₂₅ U87MG: 18.94 μM & C6: 31.76 μM), MET (IC₂₅ U87MG:34.03 μM & C6:38.40 μM), and EGCG (IC50 U87MG: 43.66 μM & C6: 43.94 μM), were found to be the lowest (U87MG: 0.54 & C6: 0.55, CI<1), thereby suggesting the synergistic effect for triple-drug combination to be the strongest. The dual-drug combinations (TMZ+MET; TMZ+EGCG; MET+EGCG) were also found to be synergistic with a CI value of U87MG: 0.80 & C6: 0.67; U87MG: 0.87 & C6: 0.72; U87MG: 0.80 & C6: 0.75, CI<1, respectively. Notably, the triple-drug combination (TMZ+MET+EGCG) exhibited the most significant synergistic effect (FIG. 3).

### Example 2—TMZ, MET, and EGCG in Combination Promote Apoptosis of Glioma Cells

To determine whether the suppression of glioma cell proliferation and the cytotoxicity observed in MTT analysis were due to induction of apoptosis or necrosis, AO/EtBr dual staining was performed. AO/EtBr are fluorescent nuclear stains, which distinguish between live and dead cells based on their membrane integrity. AO intercalates in the DNA of live cells imparting green shade to the nucleus as an indica-tor of viability. In the case of the dead cell, EtBr intercalates along with AO, resulting in an orange appearance, with an increased orange appearance corresponding to a concomitant increase in the number of dead cells and vice versa.

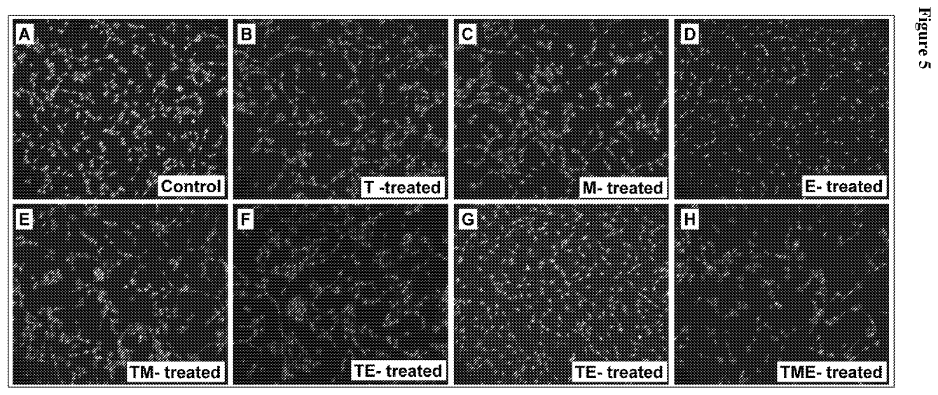

U87MG and C6 glioma were seeded at a concentration of 2×10⁵ cell/ml in 6-well tissue culture plates and were treated with various treatment groups at their respective IC₅₀ concentrations. After the treatment period, monolayer cells were stained with AO/EtBr stain (1 mg/ml) and were visualized immediately under a fluorescence microscope (Axiovert, Carl Zeiss) at 20× magnification. The images were acquired using a 495 to 515 nm filter. The results obtained in U87MG and C6 glioma cells are presented in FIGS. 4 and 5, respectively.

Results. As evident in FIGS. 4H and 5H, an elevated number of dead cells and cell shrinkage (cell disintegration) was observed in the triple-drug combination (TMZ+MET+EGCG) in both cell lines. In the dual-drug combination, TMZ+MET was found to induce apoptosis; however, cell shrinkage was not observed. Yet, in the other dual-drug combinations namely, MET+EGCG and TMZ+EGCG, the cells were found to be viable with a green nuclear region but exhibited an orange cytosol, indicating that the cells were progressing towards early apoptosis; however, the cell morphology remained unchanged (FIGS. 4E, F and G; 5E, F and G). Interestingly, in TMZ-only treated cells, a higher rate of apoptosis was observed without any change in cell morphology (FIGS. 4B and 5B). Treatment with MET alone or EGCG alone did show an anti-proliferative effect but did not produce any significant apoptotic activity (FIGS. 4C and D; 5C and D).

### Example 3—Triple Combination of TMZ, MET, and EGCG Induces Apoptosis Via the Production of Reactive Oxygen Species (ROS)

The levels of intracellular ROS in TMZ, MET, and EGCG-treated glioma cells was investigated.

Determination of ROS activity by DCF-DA. U87MG and C6 cells were seeded at 5×10³ cells/well in 96 well plates. After 24 hr of incubation, the old medium was removed and replaced with fresh media supplemented with the different treatment drugs at the IC₅₀ concentrations. After the treatment period, monolayer cells were stained with DCF-DA stain, and the absorbance was read at 530 nm spectrophotometrically (Molecular Devices Spectra-Max M5, USA). This method is commonly used in ROS investigations and is based on the application of H2DCFDA (acetylated form of DCF), which is consecutively deacetylated inside the cells by intracellular esterase. The resulting molecule is oxidized by intracellular ROS to produce a fluorescent product, DCF.

Determination of TBARS levels. TBARS (thiobarbituric acid reactive substances), by-products of lipid peroxidation, were analyzed by measuring the concentration of malondialdehyde (MDA).

The levels of MDA were measured in cell lysates using TBARS assay (Catalog No. 10009055, Cayman Chemical, Ann Arbor, USA). The concentration of lipid peroxides was calculated as an MDA equivalent using the extinction coefficient for the MDA-TBA complex of 1.56×10⁵ M−1 cm⁻¹ at 532 nm.

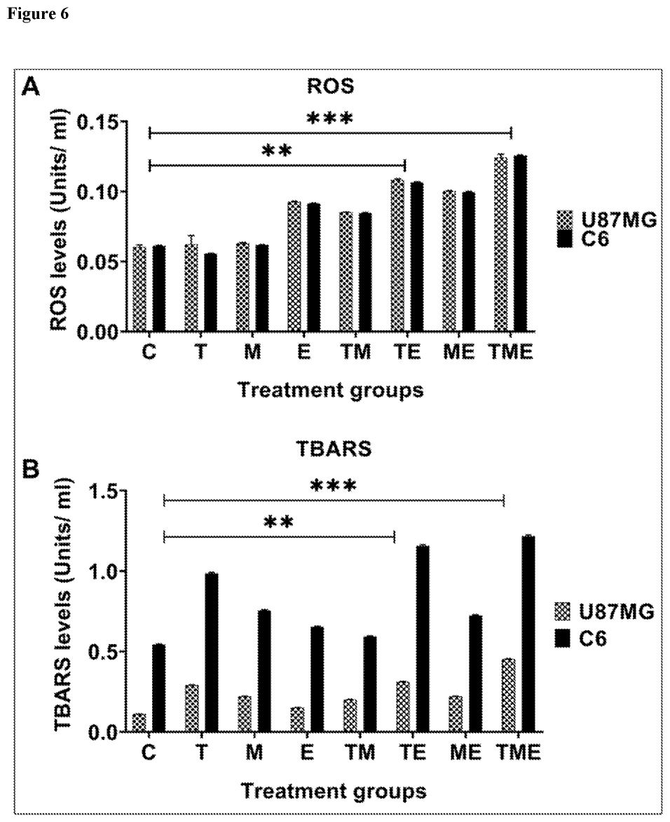

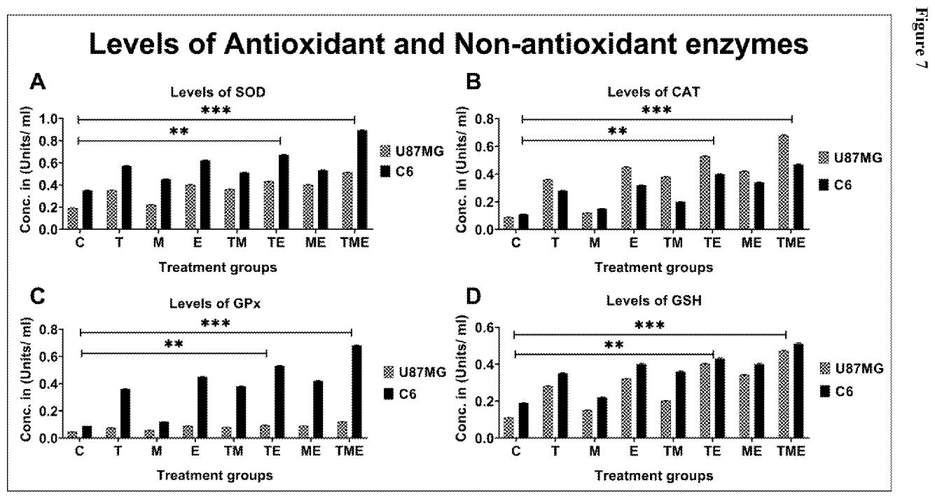

Results. The intracellular ROS levels in control-glioma and TMZ- MET- and EGCG-treated glioma cells were examined using a DCF-DA fluorescent dye assay. The fluorescence intensity increased significantly when the glioma cells were treated with the triple drug combination (p<0.01), followed by dual-drug (T-MZ+EGCG), and EGCG-treated (p<0.05) glioma as compared to control and other treatment groups. The high fluorescence intensity in these groups may be due to the increased production of ROS in the triple-drug combination. Interestingly, the production of ROS was significantly reversed in individually EGCG-treated glioma cells (FIGS. 6A and 6B). MET-alone treated glioma cells did not show much effect on ROS levels (less fluorescence), yet its combination with EGCG (MET+EGCG) significantly augmented the levels of ROS when compared with the other combinations (FIGS. 6A and 6B). These results suggest that the triple-drug combination was able to elevate intracellular ROS levels leading them to lethal oxidative stress, which can render tumor cells vulnerable to apoptosis.

To further evaluate the generation of ROS in glioma cells, TBARS levels were measured as a marker for lipid peroxidation. TBARS levels were found to be significantly elevated in the triple-drug (TMZ+MET+EGCG) (p<0.01), dual-drug (TMZ+EGCG), and EGCG-alone treated (p<0.05) glioma cells, with the triple-drug combination exhibiting the highest TBARS levels. No significant change in the levels of TBARS was observed in other treatment groups (FIGS. 6C and 6D).

### Example 4. Triple Combination of TMZ, MET, and EGCG Elevates Levels of Antioxidant Markers and Modulates Oxidative Stress Status in Glioma Cells

Antioxidant markers. The antioxidant potential of TMZ, MET, and EGCG, individually and in combination, against glioma cell was analyzed by evaluating the concentrations of the antioxidant biomarkers superoxide dismutase (SOD), catalase (CAT), glutathione peroxidase (GPx), and reduced glutathione (GSH), using a Cayman SOD Assay kit (Catalog No. 706002, Cayman Chemical, Ann Arbor, USA), Cayman CAT Assay Kit (Catalogue No. 707002, Cayman Chemical, Ann Arbor, USA), GPx Assay Kit (Catalog No. 703102; Cayman Chemical, Ann Arbor, Michigan, USA), and Glutathione Assay Kit (Catalogue No. 703002, Cayman Chemical, Ann Arbor, USA). All experiments were carried out according to the manufacturer's instructions. Three independent biological replicates were performed for each condition.

Quantitative RT-PCR analysis of antioxidant enzymes (SOD and CAT), oxidative stress marker (Nrf-2), and apoptosis markers (BCL2 and Caspase-9). Total RNA from all experimental groups was isolated using RNA Express reagent (HiMedia, India) and the respective cDNAs were synthesized using a Hi-c-DNA Synthesis Kit (HiMedia, India). Quantitative RT-PCR was carried out using a CFX 96 thermocycler (Bio-Rad, Hercules, CA) and TB Green Premix Ex Taq 1 (Takara Bio, Otsu, Japan) to detect mRNA. The specific PCR primer sequences of these genes, designed using BLAST, are listed in Table 3. Independent experiments were conducted in triplicate. The cycle threshold (Ct), representing a positive PCR result, is defined as the cycle number at which a sample's fluorescence intensity crossed the threshold automatically determined by the CFX 96.

The relative changes in gene expression were calculated with the 2^{-ΔΔCt} method, wherein ΔΔCt=(Ct_{target gene}−Ct_{β-actin}) sample−(Sample Ct_{target gene}−Control Ct_{target gene}) calibrator.

TABLE 3
Primer sequences for analyzed genes.

| | Forward Primer | Reverse Primer |
|---|---|---|
| Gene | (5′-3′) | (5′-3′) |
| SOD | ACTGGTGGTCCATGAA | AACGACTTCCAGCGTTTCCT |
| (human) | AAAGC | (SEQ ID NO: 2) |
| | (SEQ ID NO: 1) | |
| SOD (rat) | CGGATGAAGAGAGGCA | CAATCACACCACAAGCCAAG |
| | TGTT | SEQ ID NO: 4 |
| | (SEQ ID NO: 3) | |
| CAT | GCTCTTCTGGACAAGT | TTACACGGATGAACGCTAAG |
| (human) | ACAATGCTG | CTTC |
| | (SEQ ID NO: 5) | (SEQ ID NO: 6) |
| CAT (rat) | GGCAGCTATGTGAGAG | CTGACGTCCACCCTGAC |
| | CC | (SEQ ID NO: 8) |
| | (SEQ ID NO: 7) | |
| Nrf-2 | CAAATCCCACCTTGAA | CGACTGACTAATGGCAGCAG |
| (human) | CACA | (SEQ ID NO: 10) |
| | (SEQ ID NO: 9) | |
| Nrf-2 (rat) | CAAATCCCACCTTGAA | CGACTGACTAATGGCAGCAG |
| | CACA | (SEQ ID NO: 12) |
| | (SEQ ID NO: 11) | |
| BCL2 | ATCGCTCTGTGGATGA | AGAGACAGCCAGGAGAAATC |
| (human) | CTGAGTAC | AAAC |
| | (SEQ ID NO: 13) | (SEQ ID NO: 14) |
| BCL2 (rat) | CATGCTGATGTCTCTG | GATGTGATGCCTCTGCGAAG |
| | GAATCT | (SEQ ID NO: 16) |
| | (SEQ ID NO: 15) | |
| Caspase-9 | AGTTCCCGGGTGCTGT | GCCATGGTCTTTCTGCTCAC |
| (human) | CTAT | (SEQ ID NO: 18) |
| | (SEQ ID NO: 17) | |
| Caspase-9 | AGTTCCCGGGTGCTGT | GCCATGGTCTTTCTGCTCAC |
| (rat) | CTAT | (SEQ ID NO: 20) |
| | (SEQ ID NO: 19) | |
| ß-actin | AGAGCTACGAGCTGCC | AGCACTGTGTTGGCGTACAG |
| (human) | TGAC | (SEQ ID NO: 22) |
| | (SEQ ID NO: 21) | |
| ß-actin | ATCTGGCACCACACCT | AGCACAGCCTGGATAGCAAC |
| (rat) | TCT | (SEQ ID NO: 24) |
| | (SEQ ID NO: 23) | |

Oxidative stress and apoptosis marker protein expression. ELISA was performed to determine the levels of Nrf-2 protein using a Nrf-2 ELISA kit (Catalog No. E-EL-H1564 and E-EL-R1052, Elabsciences, Wuhan China, for human and rat Nrf-2, respectively. Caspase-9 protein levels were determined using a Caspase-9 ELISA kit (Catalog No. E-EL-H0663 and E-EL-R0163, Elabsciences, Wuhan China for human and rat Caspase-9, respectively.

Statistical analysis. All experiments were conducted at least three times. Data from IC₅₀ values, biochemical parameters, and RT-PCR are expressed as the mean±standard deviation (SD). Statistical analysis of all other data was conducted using t-test (un-paired and two-tailed) or one-way analysis of variance (ANOVA) in the statistical package SPSS 16.0 (SPSS Inc., USA). The post-hoc was performed for ANOVA with Dunnett's or Tukey post-hoc test. P<0.05 was considered to be statistically significant.

Results. The mean concentrations of antioxidant markers, SOD, CAT, GPx, and GSH, were found to be significantly elevated in the triple-drug treatment groups when compared with that of control (p<0.01). However, dual-drug combination (TMZ+EGCG) and individually EGCG-treated groups too significantly enhanced the level of the antioxidant enzyme system in U87MG and C6 glioma cells (p<0.05) (FIG. 7).

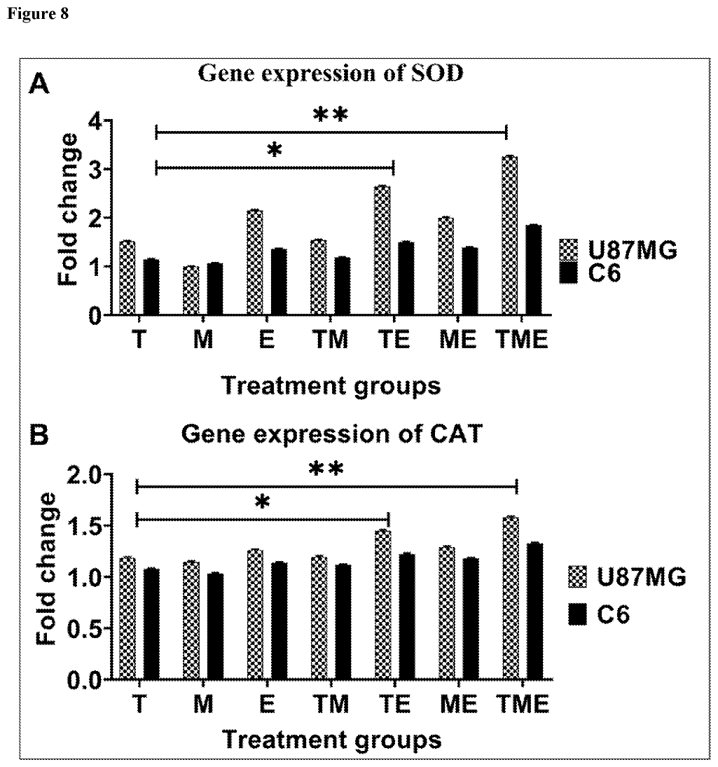

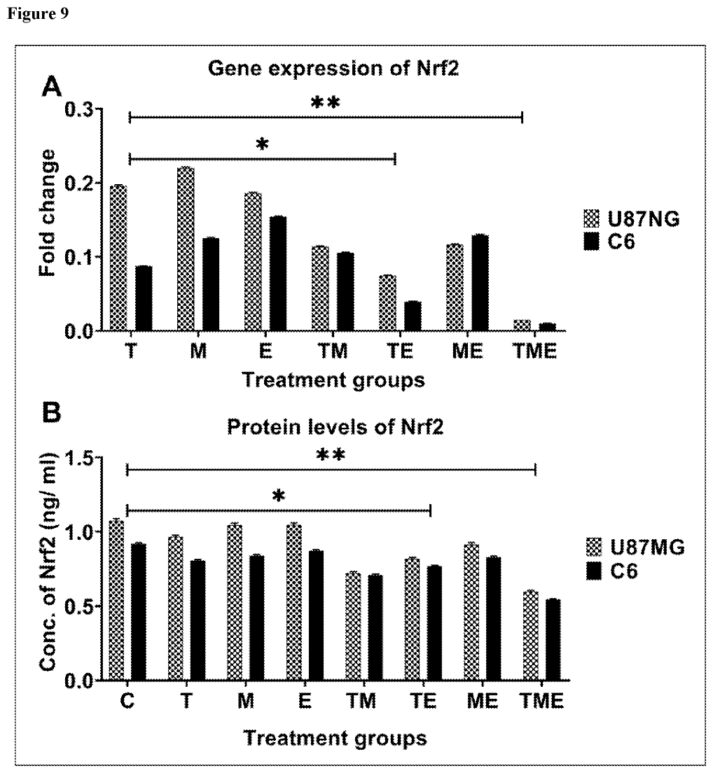

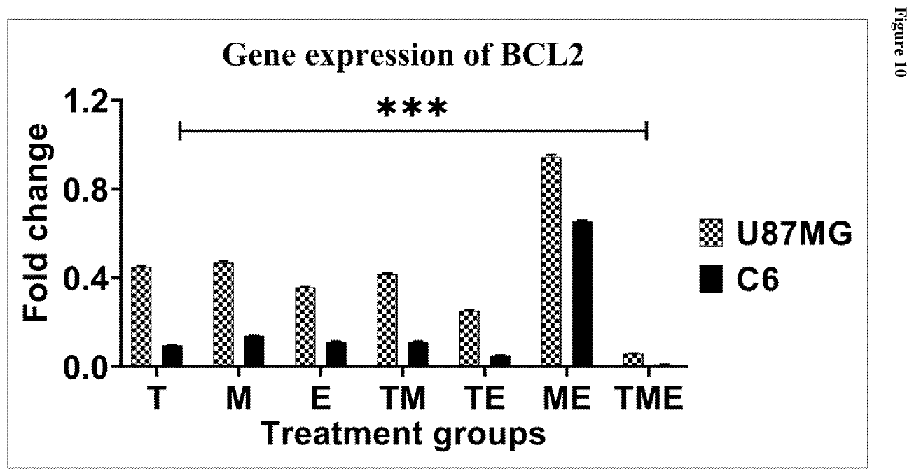

To elucidate the molecular mechanism of oxidative stress leading to apoptosis in the glioma cells treated with the TMZ, MET, and EGCG, gene expression of SOD, CAT, Nrf-2 was analyzed using RT-PCR. In the present study, we observed a significant up-regulation in the levels of mRNA transcripts of the antioxidant genes (SOD and CAT) in the triple-drug combination (TMZ+MET+EGCG) (p<0.01), dual-drug com-bination (TMZ+EGCG), and individually EGCG-treated (p<0.05) glioma cells when compared with the other experimental groups (FIGS. 8A and B). Triple-drug treated glioma cells exhibited a significant decrease in the production of free radicals as measured by the mRNA transcript levels of Nrf-2 (p<0.01), thereby indicating a reduction in the resistance to oxidative stress (FIGS. 9A and B). However, the du-al-drug combination (TMZ+EGCG) and individually TMZ-treated cells also exhibited a similar trend but were not as significant as that of the triple-drug combination. Interestingly, it was noted that individually MET and EGCG-treated cells did not have any significant effect on the levels of mRNA transcripts of Nrf-2 in glioma cells (FIGS. 9A and B).

Furthermore, we analyzed the protein expression levels of Nrf-2 using ELISA. As concomitant with the gene expression studies, triple-drug (TMZ+MET+EGCG) (p<0.01), dual-drug (TMZ+EGCG), and TMZ-treated (p<0.05) glioma cells significantly reduced the protein levels of Nrf-2, there-by modulating the expression of Nrf-2 in GBM cells (FIGS. 9C and D).

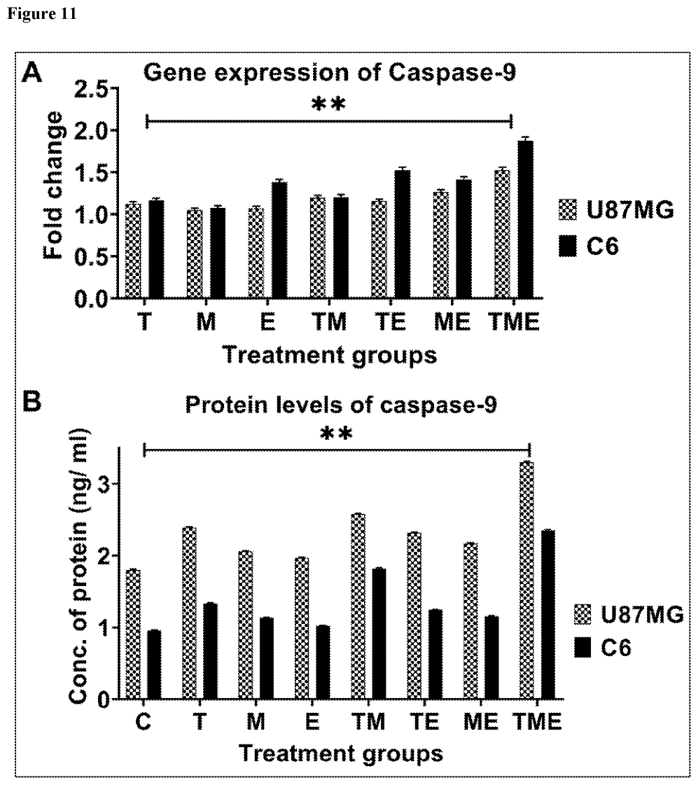

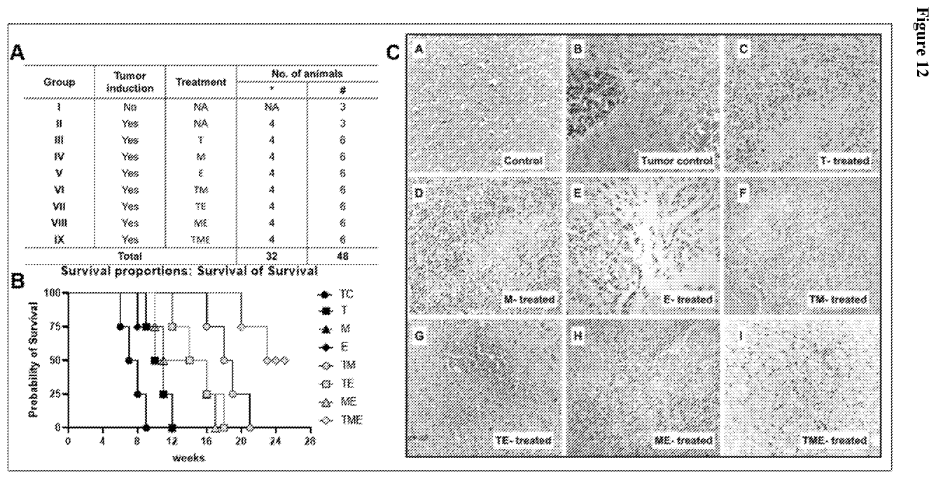

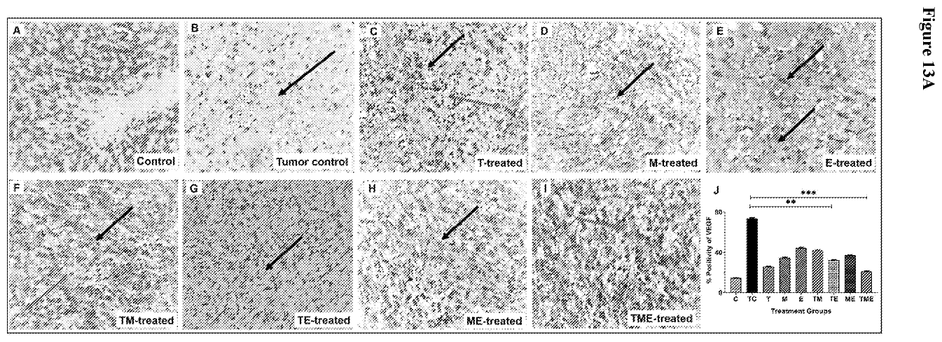

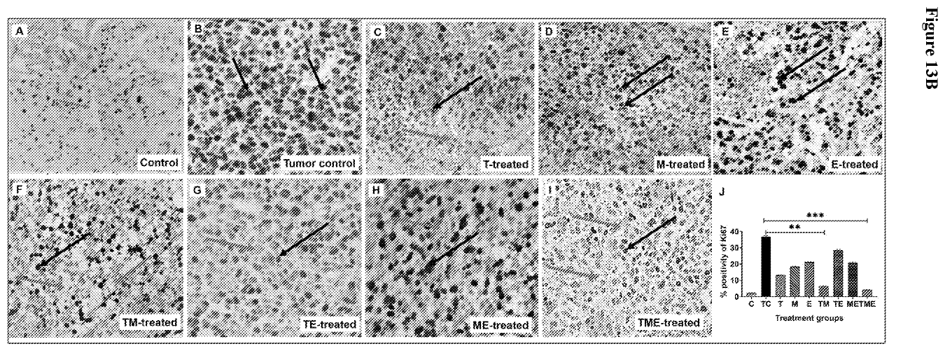

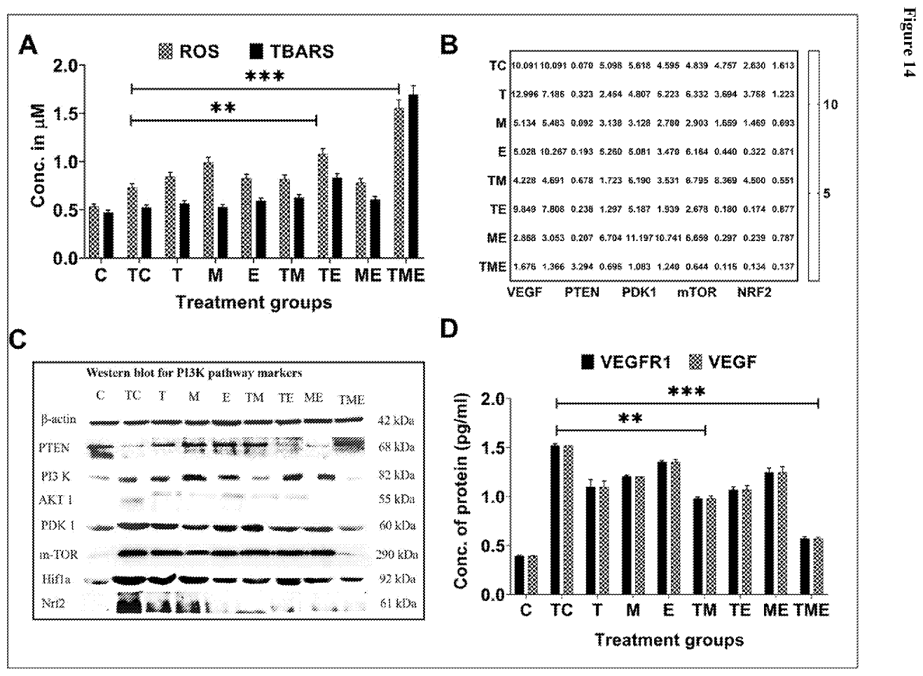

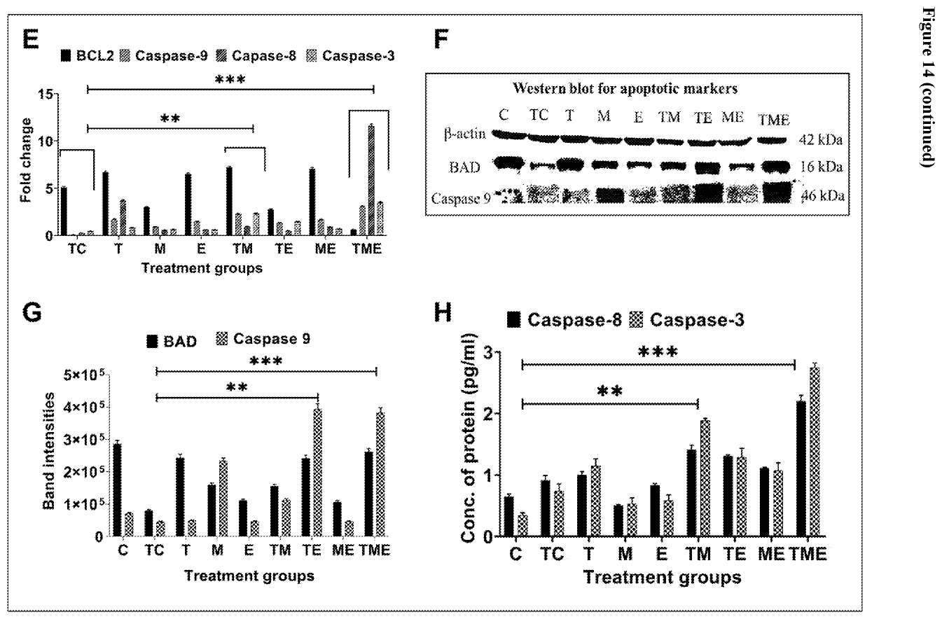

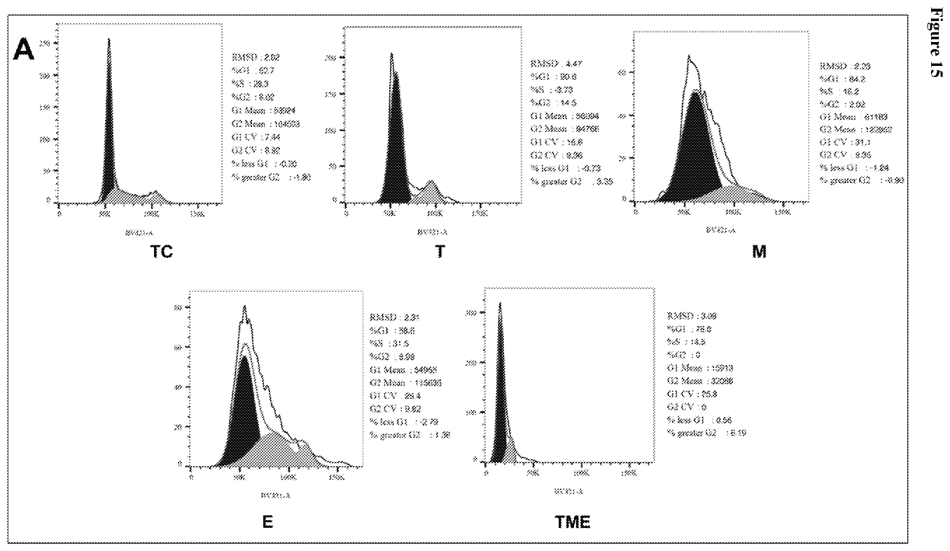

Based on the significantly reduced levels of Nrf-2 and increased ROS levels in the triple-drug treated cells, we further investigated the mechanism of apoptosis induction caused by oxidative stress in glioma cells treated with the drugs, either alone or in combination. BCL2 (anti-apoptotic) and caspase-9 (pro-apoptotic) serve as an important regulators of apoptosis in GBM cells. As depicted in FIG. 10, the intracellular mRNA expression levels of BCL2 were significantly down-regulated, while the expression levels of Caspase-9 were significantly up-regulated in the triple-drug combination (TMZ+MET+EGCG) (p<0.01) when compared with other treatment groups (FIGS. 11A and B). Though the dual-drug combination (TMZ+MET and MET+EGCG) and individually TMZ, MET, and EGCG-treated cells exhibited reduced expression of BCL2 (FIG. 10) and increased expression of Caspase-9, yet they were not found to be statistically significant (FIGS. 11A and B). Similarly, the triple-drug combination (TMZ+MET+EGCG) was found to significantly (p<0.01) increase the protein levels of Caspase-9, while other treatment groups were found to have no significant change in the protein levels of Caspase-9 (FIGS. 11C and D).

### Example 5—TMZ, MET, and EGCG Inhibit Tumor Growth In Vivo

Cell Culture. C6 rat glioma cell line (Passage no. 35) was procured from National Centre for Cell Sciences (NCCS), Pune, India and were employed for establishing the orthotopic xenograft glioma tumor. C6 glioma cells were cultured in T75 flasks containing DMEM medium supplemented with 10% FBS and 1% penicillin-streptomycin. The cultures were maintained at 37° C. in a humidified atmosphere containing 5% CO₂.

Animals. Healthy male Wistar rats (180-240 g/6 to 8 weeks old) were purchased from Manipal Academy of Higher Education (MAHE), Manipal, India. Animals were maintained in a ventilated and temperature-controlled atmosphere at 23-25° C. with a 12 Hrs light/dark cycle and had access standard food pellets and water. All the animal experiments were per-formed in accordance with the guidelines of Institutional Animal Ethics Committee (IAEC), Kasturba Medical College, Manipal Academy of Higher Education (MAHE), Manipal (IAEC/KMC/69/2019) and Mahatma Gandhi Medical College and Research Institute, Sri Balaji Vidyapeeth (Deemed to-be University), Puducherry (07/IAEC/MG/08/2019-II).

Orthotropic xenograft glioma model. Wistar rats were anesthetized with a ketamine/xylazine cocktail solution (87 mg/kg body weight and 13 mg/kg body weight) and were placed in the stereotaxic head frame. A 1 cm midline scalp incision was made, and 1×10⁶ C6 glioma cells in a volume of 3 μl phosphate buffer saline (PBS) was injected at a depth of 6.0 mm in the right striatum (coordinates with regard to Bregma: 0.5 mm posterior and 3.0 mm lateral) through a burr hole in the skull using a 10 μl Hamilton syringe to deliver tumor cells to a 3.5 mm intra-parenchymal depth. The burr hole in the skull was sealed with bone wax and the incision was closed using dermabond. The rats were monitored daily for signs of distress and death.

Study design. This study was conducted on nine series of experimental groups (N=48), namely Group I: Normal rats without tumor induction (Control C) (n=3); Group II: Tumor-control (TC) (n=3); Group III: TMZ-treated glioma-induced rats (T) (n=6); Group IV: MET-treated glioma-induced rats (M) (n=6); Group V: EGCG-treated glioma-induced rats (E) (n=6); Group VI: TMZ and MET-treated glioma-induced rats (TM) (n=6); Group VII: TMZ and EGCG-treated glioma-induced rats (TE) (n=6); Group VIII: MET and EGCG-treated glioma-induced rats (ME) (n=6); and Group IX: TMZ and Met and EGCG-treated glioma-induced rats (TME) (n=6). The dosage of the drugs alone or in combination that were administered to the rats is shown in Table 4.

TABLE 4
Treatment groups for in vivo study.

| Group | Drug | Dosage (mg/kg body weight) |
|---|---|---|
| Group II | TC | NA |
| Group III | T | 50 |
| Group IV | M | 80 |
| Group V | E | 150 |
| Group VI | TM | 50 + 80 |
| Group VII | TE | 50 + 150 |
| Group VIII | ME | 80 + 150 |
| Group IX | TME | 50 + 80 + 150 |

Treatment with drugs was commenced 20 days after orthotopic implantation of glioma cells. As the in vitro data did not reveal any significant changes in the vehicle-treated cells when compared with glioma cells, the vehicle-treated rats were not further considered for any in vivo studies. After 7 days of treatment with the drugs, animals were anesthetized and 3-4 ml of blood was collected by cardiac puncture using a 5 ml syringe. These animals were then euthanized with isosulfan followed by cervical dislocation, and the brain tissue was isolated, stored accordingly, and subjected to further studies.

Drug preparations. T, M and E, individually and in combination were prepared on each day of injection in sterile water (vehicle) at varying concentrations as listed in Table 4, respectively. The drugs were stored at 4° C. before administration and were injected within 1 hr of formulation. All the drugs were administered intra-peritoneally in a volume of 0.1 ml.

Survival statistics and toxicity studies. The survival study was carried out on a new set of thirty-two (N=32) rats that were implanted with C6 glioma cells and treated with the respective drugs as described in Table 4. After the treatment period, the animals were continuously monitored for their survival rates for a period of 25 weeks. In the survival analysis, the death of a rat in each group of treatment was taken as a break point and a survival graph was plotted using GraphPad Prism 8 (San Diego, USA). Any animal surviving a period of 25 weeks was euthanized under strict ethical guidelines, to avoid further suffering of the animal. Post-mortem, drug-induced toxicity was determined by histopathological analysis of major organs namely liver, lungs, spleen, kidney and pancreas.

Heamatoxylin and eosin staining. After treatment for 7 days, the rats were euthanized, and the brain tissues of the experimental groups were collected and fixed in 4% PBS-buffered paraformaldehyde followed by embedding in paraffin. For haematoxylin and eosin (H & E) staining, paraffin blocks were sectioned by 5 μm thickness. Slides were then stained with H & E and were observed under 20× magnification with a compound microscope (Axiovert, Carl Zeiss, USA) to take digital photographs.

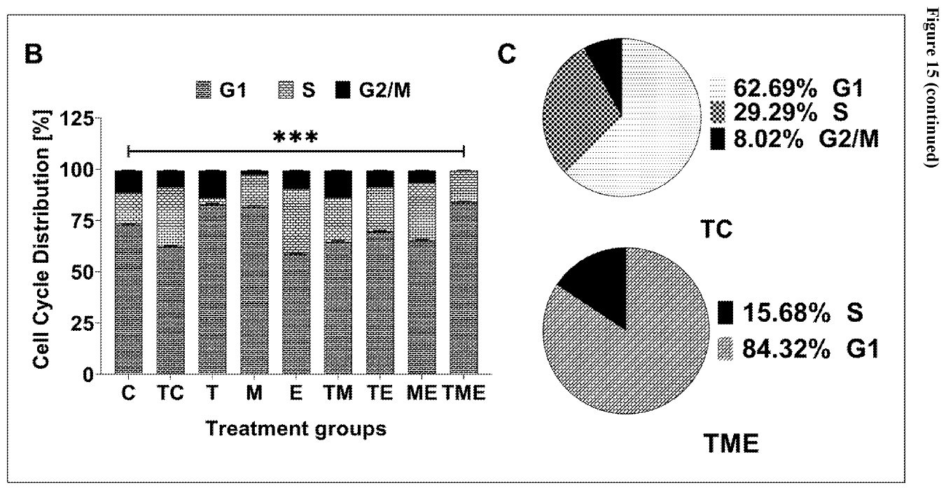

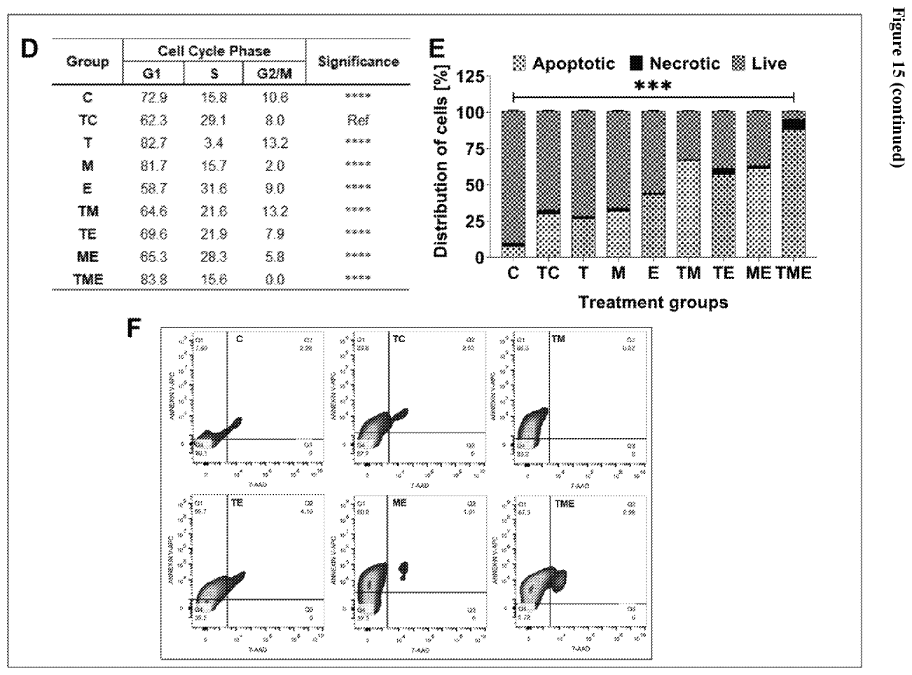

Results. To study the beneficial mechanism of TMZ, MET, and EGCG, individually and in combination, in terms of prolonging prognosis and survival rate, a survival study was carried out. The animal used in this study are depicted in FIG. 12A (* Survival study; #Experimental studies).

At the end of the study, the rats were sacrificed, and the survival results were interpolated using Kaplan-Meier Survival graph. As shown in FIG. 12B, treatment of the rats with the triple-drug combination (TME) significantly improved tumor suppression and prolonged the duration of survival (>25 weeks) in 50% of the treated animals, relative to the effect observed with the other experimental groups. The median survival rate of the triple-drug treated animals (TME) (24 weeks) is significantly higher than the tumor-control group (7.5 weeks) (P<0.0001). Further, among the dual-combinations administered, TM and TE showed a slightly better median survival rate of 18.5 and 15 weeks respectively relative to ME group (13.5 weeks). Conversely, delivery of either of these drugs as individual regime did not correlate with significant improvement in the survival of rats when compared with the triple-drug combination. These results demonstrated that combined delivery of T, M and E produced superior survival benefit and prolonged the survival of animals.

Following glioma implantation and successive treatment of the glioma-induced rats with the drugs, both individually and in combination, histopathological analysis of vital organs was carried out by H&E staining. Interestingly, no significant pathological changes were observed in any of the organs obtained from the drug-administered groups when com-pared with the control group. This result highlights that the none of the treatments induced any organ-based toxic effects.

Following assessment of the survival benefit bestowed within the combinatorial treatment, we were interested in determining their effect on pathological changes in the brain tissues.

Histological analysis by H & E staining depicted a considerable decrease in tumor cells in the sections obtained from the triple-drug combination (TME) when compared with the other treatment groups and control. While the tumor-control (TC) rats exhibited intense cellularity and extensive hemorrhage in blood vessels (FIG. 12C-b), the cellularity and hemorrhage were drastically reduced in the rats treated with the triple-drug combination (FIG. 12C-i). In fact, the tumor cell density in the triple-drug treated tissues were almost similar to the non-tumor brain regions. Among the dual-drug treatment groups, TM and TE portrayed a slight reduction in the whorled structures and reduced cell density but were not as effective as the triple-drug combination (FIG. 12C-f & g). Though treatment with these drugs as individual regime had relatively modest effect with decreased hemorrhage, they were not as effective as the triple-drug combination. These results shed light on the fact that besides prolonging survival, the triple-drug com-bination can effectively reduce the extent of tumor, which was grossly observed in a significant manner.

### Example 6—Combined TMZ, MET, and EGCG Reduced Cell Proliferation and Angiogenesis

Immunohistochemistry. For immunohistochemistry (IHC) analysis, 4 μm thick tissue sections were deparaffinized in xylene and hydrated by immersing in a series of graded ethanol concentration (100%, 95%, 75% and 50%). Endogenous peroxidase activity was blocked by incubating sections in 3% H₂O₂ solution prepared in methanol at room temperature for 10 minutes and were washed with PBS twice for 5 minutes each. Antigen retrieval was performed by immersing the slides in 300 ml of retrieval buffer (10 mM citrate buffer, pH 6.0) for one hour at 100° C. and were rinsed twice in PBS. Sections were then incubated with approximately 100 μl diluted primary antibodies of Ki67 (1:100 dilution, Elabscience, Wuhan, China) and Vascular Endothelial Growth Factor (VEGF) (1:100 dilution, Elabscience, Wuhan, China) for 30 minutes at room temperature. The slides were then washed twice with PBS and were then incubated with approximately 100 μl of diluted secondary antibody (Cat No. E-IR-R213, Elabscience, Wuhan, China) at room temperature for 30 minutes. Slides were then washed in PBS for 5 minutes, followed by incubation with approximately 100 μl diluted Horse Radish Peroxidase (HRP) conjugate for 30 minutes. The slides were then incubated in 100 μl diluted 3,3′-Diaminobenzidine (DAB) chromogen substrate for the development of color and the sections were counter-stained by haematoxylin for 1-2 minutes. The slides were washed under running tap water to remove excess stain and dried at room temperature. The sections were then dehydrated with series of graded ethanol concentration (95%, 95%, 100%, and 100%) and slide mounted. The color of the antibody staining in the tissue sections was observed under 40× magnification in a compound microscope (Axiovert, Carl Zeiss, USA). The staining intensities were quantified using IHC Profiler, a plugin in ImageJ software, to determine the H-score (Histo score).

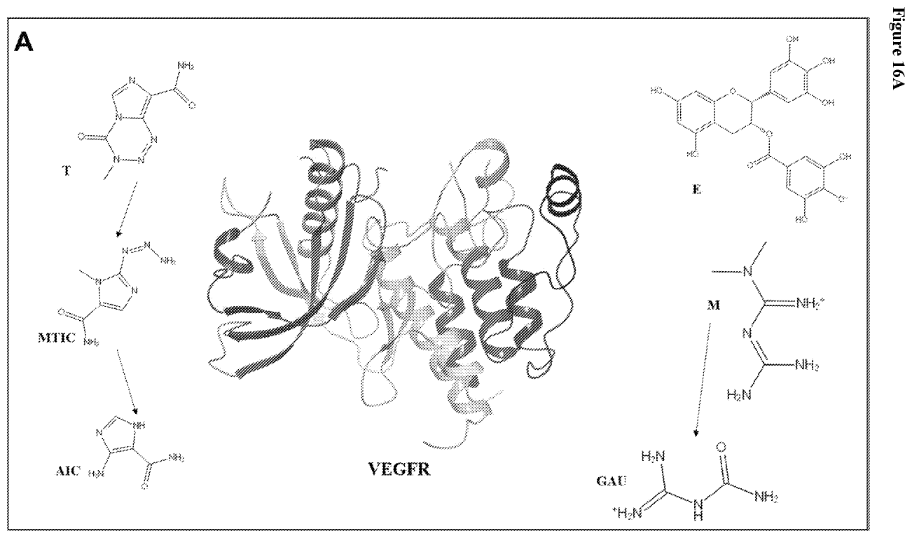

Results. Considering the significant anti-invasive effect of the triple-drug combination, we then evaluated the expression levels of VEGF (angiogenic marker) and Ki67 (proliferation marker) in the tumor sections of the experimental groups. Assessment of cytoplasmic positivity for VEGF was significantly reduced in tumors treated with the triple-drug combi-nation (21.12%) (specified by red arrows in FIG. 13A-i), when compared with tumor-control group, which exhibited the highest (73.58%) level of cytoplasmic positivity (black arrows indicated in FIGS. 13A-b). TE, T-alone and E-alone depicted a moderate cytoplasmic positivity, as evident by the H-score of, 32.34, 25.83 and 44.45% respectively (FIG. 13A-j).

In addition, Ki67 exhibited a lower nuclear positivity rate in the tumor-induced rats that received the triple-drug combination therapy (4.21%) (as specified by red arrows in FIG. 13B-i). The highest nuclear positivity rate was noticed in tumor-control rats (36.73%) (as indicated by black arrows in FIG. 2B-b). However, the dual-combination therapy of TM (6.42%), T-alone (13.14%) and M-alone (18.36%) showed significantly lower nuclear positivity of Ki67 expression but were not as significant as the triple-drug combination (FIG. 13 2B-j). Conversely, the other treatment groups did not induce any significant changes in the nuclear positivity of Ki67 expression (P<0.01).

### Example 7—Combined TMZ, MET, and EGCG Increased Levels of ROS, Promoted Apoptosis, and Increased Apoptotic Markers

In most cancers, angiogenesis is vital for tumor growth and proliferation, which is often facilitated by reduced levels of ROS. Conversely, elevated ROS levels have been reported to reduce tumor progression and inhibit angiogenesis. Therefore, to assess whether the drugs induced oxidative stress, we measured the intracellular ROS generation, extent of apoptosis, and apoptotic marker expression after treatment with TMZ, MET, and EGCG, alone and in combination.

Measurement of ROS using a DCF-DA assay. Intracellular ROS was determined by measuring the DCF fluorescence intensity as discussed above in Example 3. In these studies, 1×10³ astrocytes isolated from each experimental group were seeded on a 96-well plate. The cells were then stained with 100 μl of DCF-DA (100 μM) and incubated at 37° C. for 15 min. The relative fluorescence intensity of oxidized product of DCF-DA was measured by reading the absorbance at 530 nm in a spectrophotometer (Molecular Devices Spectra-Max M5, USA).

Measurement of antioxidant, non-antioxidant enzymes, and lipid peroxidation. Biochemical analysis was performed on the brain tissue lysates to measure (i) SOD activity using an SOD Assay kit (Cat. No. 706002); (ii) Catalase activity using a CAT Assay Kit (Cat No. 707002); (iii) GPx activity by GPx Assay Kit (Cat No. 703102); and (iv) Glutathione activity with GSH Assay Kit (Cat. No. 703002). For measurement of malondialdehyde (MDA) levels, which is a byproduct of lipid peroxidation, a TBARs assay kit (Cat. No. 10009055) was employed. All biochemical parameters were performed using these kits available from Cayman chemical, Ann Arbor, USA, and all analyses were performed according to the manufacturer's protocol. Three independent biological replicates were performed for each assay.

Cell lysis and protein quantification. For protein isolation, 500 mg of brain tissues of all the experimental groups were washed with 0.9% NaCl, 5 times each, to remove contaminated blood and was fully grilled in liquid nitrogen. Two ml of RIPA protein extraction buffer (20 mM Tris-HCl (pH 7.5), 150 mM NaCl, 1 mM sodium ethylenediamine tetra acetate (EDTA), 1 mM EDTA, 1% NP-40, 1% sodium deoxycholate, 2.5 mM sodium pyrophosphate, 1 mM β-glycerophosphate, 1 mM Na3VO4, 1 μg/ml leupeptin) was added to the brain tissues, crushed and then centrifuged at 12000×g for 15 minutes at 40° C. The lysates were then collected, centrifuged again at 12000×g for 15 minutes at 40° C. The protein extract collected as a supernatant was then quantified using Bradford method.

Western blotting. Following quantification, 40-100 μg of the total protein were separated by sodium dodecyl sulphate polyacrylamide gel electrophoresis (SDS-PAGE) and then transferred onto polyvinylidene difluoride (PVDF) membrane (Millipore, Germany) by the wet transfer method. The membrane was blocked with 5% skim milk at room temperature and then incubated with the indicated primary antibodies against β-actin, Nrf-2, hypoxia inducible factor-1 alpha (HIF-1α), PI3K, pyruvate dehydrogenase kinase-1 (PDK1), pAKT1, phosphatase and tensin homolog (PTEN), Caspase-9, pmTOR and BAD (Elabsciences, Wuhan, China) on an orbital shaker at 40° C. overnight. After washing three times with tris-buffered saline/tween-20 (TBST), the blots were incubated with HRP-conjugated secondary antibodies (Elabsciences, Wuhan, China) for 1 hr on an orbital shaker at room temperature. Each immune complex was detected by Enhanced Chemiluminescence (ECL) Substrate (Santa Cruz, Texas, USA) and was visualized by the Chem-doc (Pearl® Trilogy/LI-COR, USA). Quantitative analysis for Western blot was made by ImageJ® software.

Enzyme-Linked Immunosorbent Assay (ELISA). According to the manufacturer's protocol, for the isolated protein samples from the tissue homogenates of all the experimental groups, ELISA was performed for (i) VEGF (Cat. No. E-EL-R1058); (ii) VEGFR-1 (Cat. No. E-EL-R0911); (iii) Caspase-8 (Cat No. E-EL-R0280) and (iv) Caspase-3 (Cat No. E-EL-R0160). All the ELISA kits were obtained from Elabsciences, Wuhan, China. The concentration levels of the above-mentioned proteins were calculated from a standard curve obtained.

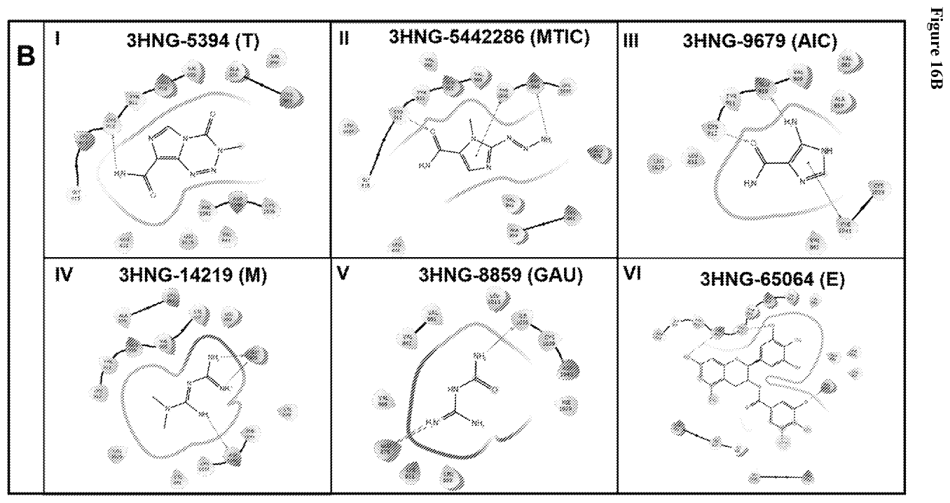

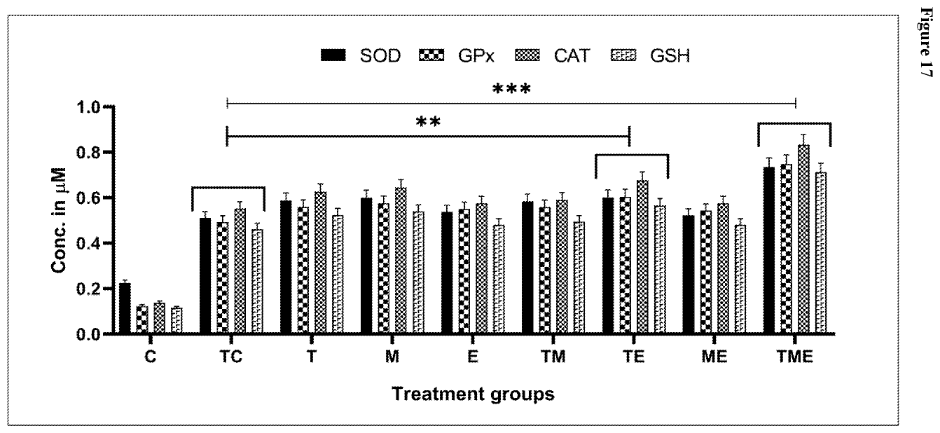

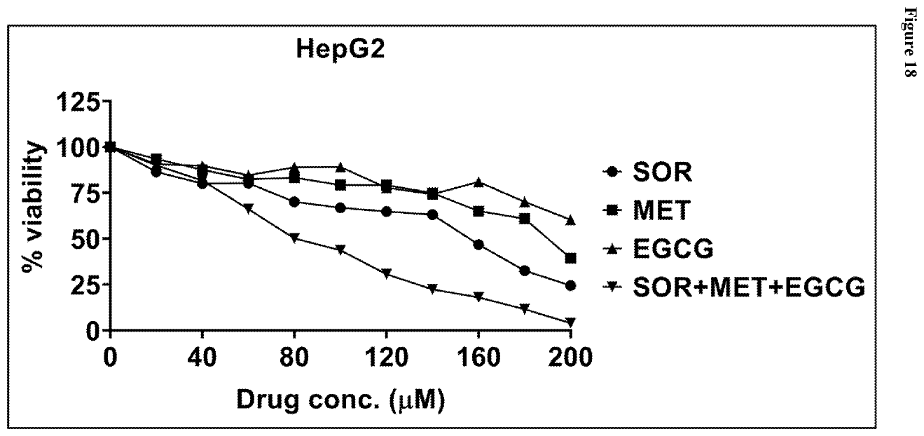

Results—The triple drug combination enhanced ROS levels. Treatment of glioma-induced rats with the triple-drug combination was associated with a significant increase in ROS (P<0.001) as compared to that of tumor-control rats (FIG. 14A). Further, the dual-drug combination (TE) was also shown to exhibit a profound increase in the ROS levels (P<0.01) but were not as significant to triple-drug combination (FIG. 14A). Although the other treatment groups exhibited an increase in the intracellular ROS, there were not significant (FIG. 14A). These results demonstrate that the triple-drug combination treatment triggered ROS production in glioma-induced rats.

ROS formed in tumor cells results in lipid peroxidation and subsequently increases MDA. MDA can be directly quantified by determining the levels of TBARS, a marker of lipid peroxidation FIG. 14A depicts the concentration of TBARS in the brain tissues of all the experimental groups. In the present study, the levels of TBARS were significantly enhanced in the triple-drug combination (P<0.001) and the dual-drug combination of TE (P<0.01) (FIG. 14A) when compared to tumor-control rats. However, the levels of TBARS were not more significant as triple-drug combination than the other experimental groups.

Results—The triple drug combination enhanced antioxidant enzyme expression. The effect of TMZ, MET, and EGCG, alone and in combination, on the antioxidant and non-antioxidant enzymes SOD, CAT, GPx and GSH was also investigated, as enhancement of these antioxidant enzymes would indicate an increased accumulation of super oxides, such as O2- and H2O—, leading to enhanced oxidative stress. Interestingly, the activity of all the antioxidant and non-antioxidant enzymes was significantly enhanced in the triple-drug combination (P<0.001) treated rats when compared with that of tumor-control rats. However, a similar effect was observed in the dual-drug combination (TE) and E-alone (P<0.01) treated rats, but was not as significant as triple-drug combination treated rats. Since glioma is often associated with superoxide- and peroxide-mediated chemo-resistance to T, co-administration of M and E significantly enhanced the susceptibility of glioma cells to T via elevating the ROS levels, enhancing lipid peroxidation and antioxidant potential.

Results—The triple-drug combination reduced antioxidant defense. The expression of some of the antioxidant defense genes mentioned above are regulated by the transcription factor Nrf-2 under hypoxic conditions, allowing cells to regulate the oxidative stress-mediated ROS species. This is brought about by binding of Nrf-2 to the promoter regions of the antioxidant response elements, which in turn reduces the levels of ROS in the cells. The formation and accumulation of ROS in hypoxic cells is a hallmark of hypoxia involved in blood brain barrier (BBB) dysfunction. In association with the production of ROS, several factors associated with hypoxia are also activated or stabilized. In particular, HIF-1α is stabilized and stimulates the transcription of genes involved in various processes such as angiogenesis, cell proliferation, inflammation, or cancer. Both Nrf-2 and HIF-1α are well-established as mediators of resistance to anticancer therapies. Therefore, simultaneously targeting these pathways would represent an attractive approach for therapeutic development. Interestingly, in the present study, the triple-drug combination significantly attenuated the gene expression levels of Nrf-2 and HIF-1α, thereby deactivating the antioxidant defense mechanism in tumor cells (P<0.0001). In addition, treatment with dual-drug combination, TE, and E-alone also exhibited reduced expression of these genes but were not as potent as triple-drug combination (FIG. 14B). These results show that the toxicity effect of this triple-drug combination is directly related to its ability to inhibit antioxidant defense and trigger apoptosis via inhibition of Nrf-2 and HIF-1α.

Results—The triple drug combination inhibits the PI3KAKT GSK3&Nrf-2 signaling pathway. Because there was a reduction in the gene expression levels of Nrf-2 and HIF-1α in glioma cells from rats treated with the triple-drug combination, the effect of the triple combination on signaling pathways upstream of Nrf-2 and HIF-1α were assessed. Nrf-2 and HIF-1α are targets for VEGF/PI3K/AKT and GSK30, wherein P-AKT and P-GSK30 promote the separation of Nrf-2 from Keap1, thereby leading to the translocation of Nrf-2 into the nucleus. GSK3β is a substrate of the PI3K pathway that is constitutively active in unstimulated cells and is known to participate in the protective cellular response to oxidative stress. Thus, VEGF/PI3K/AKT/GSK3β signaling pathway may be one of the key regulators of cell survival. PTEN, which is the downstream molecule of this pathway, acts as a tumor suppressor by inhibiting tumor cell growth and enhancing cellular sensitivity to apoptosis. Loss of PTEN activity leads to the permanent PI3K/AKT pathway activation. As shown in FIG. 14B, the triple-drug combination significantly (P<0.0001) reduced the gene expression levels of VEGF, VEGFR, PI3K, PDK1, AKT1, mTOR and GSK3β in glioma cells from treated rats, while significantly (P<0.0001) enhancing the expression of PTEN, when compared with glioma cells from the tumor-control and other treatment groups. Similarly, the dual-drug combinations TM and TE significantly (P<0.001) reduced the levels of VEGF, VEGFR, PI3K, PDK1, AKT1 and mTOR, but not to the extent of triple-drug combination. Remarkably, these dual-drug combinations mentioned did not have any significant effect on the levels of PTEN. However, glioma cells from the other treatment groups did not show any significant changes in the gene expression levels of the PI3K signaling pathway when compared to the tumor-control (FIG. 14B). From this, it was evident that the triple-drug combination significantly reduced glioma proliferation by inhibiting the Nrf-2/HIF-1α-mediated PI3K/GSK3β signaling pathway.

Results—The triple drug combination synergistically modulated activation of the PI3KAKT GSK3β/Nrf-2 pathway in glioma-induced rats. In order to determine if the results observed from the gene expression studies correlated with protein expression levels, the protein levels of VEGF/PI3K/AKT/GSK30/Nrf2/HIF-1α pathway components were assessed by immunoblotting and ELISA. The results revealed that the triple-drug combination markedly inhibited the protein expression of VEGF, VEGFR, PI3K, pAKT1, PDK1, pmTOR, Nrf-2 and HIF-1α when com-pared to that of tumor-control (P<0.001) (FIG. 14C); while significantly enhancing the expression of PTEN. This was in concordance with the gene expression analysis. Likewise, the dual-drug combination (TM and TE) also significantly decreased the levels of VEGF and VEGFR (P<0.01) (FIG. 14D) but was not as effective as triple-drug combination. The dual-drug treatment (TM) reduced the protein expression of PI3K (P<0.01), while the levels of pAKT1 were significantly reduced by the dual-drug combination of TM and TE as well as in the individual treatment of M. In addition to the triple-drug combination, the individual treatment with M significantly reduced the levels of pmTOR, indicating a moderated cell growth and proliferation. However, the dual-drug combination (TM, TE and ME) significantly reduced the levels of Nrf-2 but did not have any significant effect on the levels of HIF-1α (P<0.01). Surprisingly, individual treatment with E significantly reduced the levels of both Nrf2 and HIF-1α (P<0.01) (FIG. 14C). These results demonstrated that the triple-drug combination inhibited glioma cell growth via VEGF/PI3K/AKT/GSK30/Nrf2 signaling pathway.

Results—The triple drug combination promoted expression of pro-apoptotic genes and inhibited expression of anti-apoptotic genes. It has been reported that increased oxidative stress and reduced proliferation via inactivation of PI3K/AKT/mTOR pathway is often linked to the induction of apoptosis in glioma tumors. Hence, to gain further insights on the effect of TMZ, MET, and EGCG on induction of apoptosis, following their efficacy on oxidative stress and proliferation, the gene and protein expression of the mediators of the intrinsic pathway of apoptosis namely, BAX, BAD, caspase-9, 8 and the executioner caspase-3, was assessed in the tumor tissues of the treated rats. As expected, the gene expression results demonstrated that the triple-drug combination (TME) significantly reduced the levels of BCL2 and enhanced the expression levels of BAX, BAD, caspase-9, -8 and -3 when compared to that of tumor-control group (P<0.0001) (FIG. 14E-H). The dual-drug combination of TM treatment significantly (P<0.001) reduced the levels of BCL2 and enhanced the levels of BAX, BAD, caspases-9, 8 & 3 (P<0.001) (FIG. 14E-H). A similar trend in the expression levels of apoptotic proteins was observed by Western blot and ELISA, however, from the western blot results it was noted that T had a significant role in the elevating the levels of BAD, as BAD levels in both T and TME groups were almost equal. From this, it is evident that the triple-drug combination of TME induced apoptosis by activating the BAD/BAX and caspase complex, thereby suggesting a reduced proliferation and induction of apoptosis at gene and protein level.

### Example 8—Effect of TMZ, MET, and EGCG on Cell Cycle and Apoptosis of Glioma Cells

To determine if the inhibition of glioma proliferation and apoptosis was accompanied by alterations in the cell cycle pattern, cell cycle analysis was performed in the glioma cells isolated from glioma-induced rats.

Cell cycle analysis. For cell cycle analysis, 1×10⁶ isolated astrocytes from the brain tissues of each experimental group were rinsed with ice-cold PBS and re-suspended in 100% methanol at 4° C. for 40 minutes. The astrocytes were then pelleted via centrifugation at 2000 rpm for 5 minutes, resuspended in 500 μl of PBS containing 0.1% triton X-100 and 22 μg of 4′,6-diamidino-2-phenylindole (DAPI), and incubated in dark for 30 minutes at 25° C. The astrocytes were fully resuspended and the cell cycle pattern was analyzed using BD Celesta Flow Cytometry (Becton-Dickinson, California, USA). The results were further analyzed and quantified with FlowJo 7.6 software (Beckman Coulter, CA, USA).

Annexin V/7′AAD assay. The percentage of apoptotic cells was determined by flow cytometry to measure the apoptosis after staining with an Annexin V and 7-amino-actinomycin D (7′AAD) kit (Annexin V-FITC-AAD Kit, Becton-Dickinson, USA). Briefly, 1×10⁶ astrocytes isolated from each experimental group were harvested and washed twice in ice-cold PBS. The astrocyte pellets were resuspended in Annexin V binding buffer and incubated on ice for 5 minutes. The cells were then stained with 7′AAD and incubated for 5 minutes. The rate of cell apoptosis was determined using a Cyto-FLEX S Flow Cytometer and analyzed with FlowJo 7.6 software (Beckman Coulter, CA, USA).

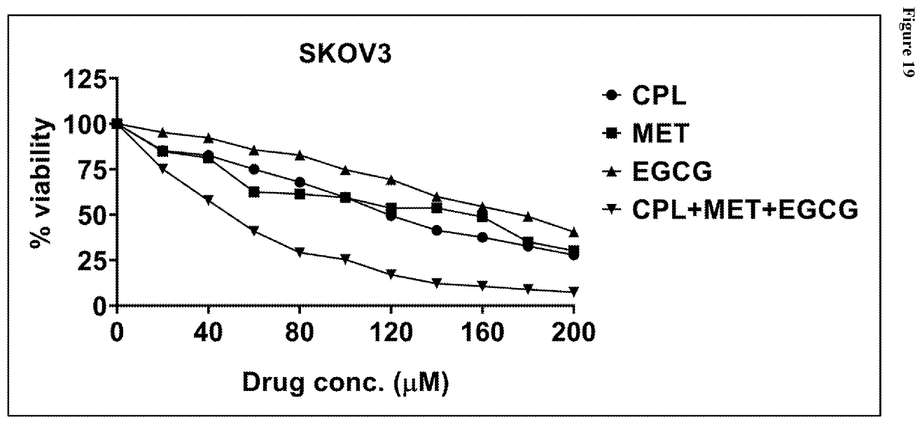

Results—Triple drug combination alters the cell cycle distribution in glioma cells. Compared to tumor-control cells, the triple-drug combination (TME) significantly enhanced the fraction of non-proliferating glioma cells (G1 phase) by 21.5% (62.3% vs. 83.8%) while significantly decreasing the percentage of proliferating cells (G2/M phase) by 8% (FIG. 15A-D). The triple-drug combination significantly reduced the number of cells in S phase by 13.5% (29.1% vs. 15.6%) when compared to the tumor-control group (P<0.0001; FIG. 15D). Further, treatment with TMZ and MET TM resulted in a significant increase in the number of cells in G1 phase, however not much effect was observed with the proportion of the cells in S-phase when compared to that of TC group (P<0.0001). In view of this analysis, it was clear that, besides inducing oxidative stress-mediated inactivation of PI3K/AKT/mTOR pathway, the combinatorial treatment of TMZ, MET, and EGCG significantly arrested the cells at G1 phase, thus leading to apoptosis (FIG. 15A-D).

Results—Triple drug combination increased the number of apoptotic glioma cells. Because combined TMZ, MET, and EGCG induced oxidative stress-mediated inactivation of the PI3K/AKT/mTOR pathway and promotion of apoptosis, the underlying mechanism by which TMZ, MET, and EGCG induced cell death was investigated. Astrocytes isolated from various treatment groups were analyzed by Annexin V/7′AAD staining and flow cytometric analysis as described above. It is well-known that Annexin V binds early apoptotic cells (Q1 in FIG. 15F), whereas 7′AAD binds late apoptotic cells (Q3). Further, the population of cells which bind both Annexin V and 7′AAD are considered to be necrotic (Q2). Live cells are found in Q4. The results showed that compared to TC, the triple-drug combination significantly enhanced the number of apoptotic (30% vs. 87%) and necrotic cells (2.5% vs. 7%) by 57% (Q1) & 4.5% (Q2) respectively and significantly reduced the number of live cells (68% vs. 6%; Q4) (P<0.001) (FIGS. 15E and 15F). Similarly, the dual-drug combination of TM increased the level of apoptotic cells by 36.5% and significantly reduced the number of live cells by 34.5% as compared to the tumor-control group (P<0.01; FIG. 15F). These results confirm that TMZ, MET, and EGCG in combination promotes cellular death in glioma cells via induction of apoptosis.

### Example 9—Molecular Docking Analysis

To perform molecular docking studies, an evaluation version of Schrödinger software package was used (Schrödinger Release 2020-3: Maestro, Schrödinger, LLC, New York, NY, 2020). The crystal structure of human VEGFR1 (3HNG) having a resolution of 2.7 Å was retrieved from protein data bank. Prior to initiating the docking protocol, the protein structure was minimized using the Protein Preparation Wizard with optimal potential to create the liquid simulation (OPLS)-2005 force field (FF). Potentially occurring stereochemical short contacts in the protein structure were removed during the protein preparation. Subsequently, water molecules without any contact were removed followed by addition of hydrogen atoms to the structure, principally at the sites of the hydroxyl and thiol hydrogen atoms, in order to correct ionization as well as tautomeric states of the amino acid residues. The 2D structure of drug molecules TMZ (T) (Pubchem ID: 5394) and its metabolites 3-methyl-(triazen-1-yl) imidazole-4-carboxamide (MTIC) (Pubchem ID: 54422836) and 5-amino-imidazole-4-carboxamide (AIC) (Pubchem ID: 9679), MET (M) (Pubchem ID: 14219) and its metabolite Guanylurea (GUA) (Pubchem ID: 8859) and EGCG (E) (Pubchem ID: 65064) were downloaded from PubChem in .mol file and prepared using the ligprep module (FIG. 16). Prior performing docking protocol using Glide standard precision (SP), the binding site pocket and grid for docking were generated using the ligand bound to the native X-ray crystallographic structure of 3HNG.

The protein structure (FIG. 16A) after minimization was found to be satisfactory to proceed further with the docking analysis. The outcome of VEGFR1 interaction with selected drug molecules was evaluated using the Glide score. FIG. 5B shows the interaction profile of selected drug molecules with VEGFR1. Based on the glide score, of the six different molecules interacting with 3HNG, it was noted that compound T ranked first (glide score, −7.938 kcal/mol) with two hydrogen interactions, one between Cys912 and the oxygen atom at the fourth position of the ligand's tetrazine ring, and a second interaction between Asp1040 and the NH2 at the eighth position of the ligand's imidazole ring, with a bond length of 1.68 and 2.28 Å, respectively.

MTIC ranked second (−6.829 kcal/mol) and demonstrated two hydrogen bond interactions, one between Cys912 and the ligand's carboximide group, and the second between Asp1040 and the ligand's aminodiazenyl moiety with a bond length of 1.8 and 2.11 Å, respectively. Furthermore, a π-π interaction was noted between the benzene ring of Phe1041 and the ligand's imidazole ring with a bond length of 4.72 Å.

AIC ranked third (−5.736 kcal/mol), and exhibited two hydrogen bond interactions, one between Val907 and the NH of the carboxamide moiety, and second hydrogen bond between Glu878 and the NH of the imidazole ring with a bong length of 2.53 and 1.9 Å, respectively. Also, an n-cation inter-action between Lys 861 and ligand's imidazole ring was noted with a bond length of 4.32 Å.

EGCG (E) demonstrated three hydrogen bond interactions, one each between the hydroxyl group and the carbonyl group of the ligand's trihydroxybenzoate moiety with Asp1040 and Arg1021 of 3HNG, with a bond length of 2.19 and 1.81 Å, respectively. A third hydrogen bond interaction was noted between the hydroxyl group of the trihydroxyphenyl attached to the dihydrochromenyl moiety and Asp807, with a bond length of 2.0 Å. In addition, an n-n interaction was observed between the benzene ring of the dihydrochromenyl moiety and the imidazole ring of His1020 with a bond length of 5.44 Å.

Compound GAU demonstrated three hydrogen interactions, two of which were noted between the ligand's NH2 group of the carbamoyl-amino portion, and the active site residues His1020 and Asp1040, with a bond length of 1.75 and 1.63 Å, respectively. The third bond was between the ligand's NH group and Asp1040 with a bond length of 2.27 Å.

Finally, of the six compounds investigated for interaction, it was noted that MET revealed formation of two hydrogen bonds, one between Glu878 and the second between Asp1040 with a bond length of 1.85 and 2.28 Å, respectively. The details of all the drugs along with their active metabolites and their respective glide scores are given in Table 5.

TABLE 5
Interaction profiles of experimental compounds with 3HNG.

| | | | | Glide Score | |
|---|---|---|---|---|---|
| SI No. | Drug | PubChem ID | Description | (kcal/mol) | Interactions |
| 1 | T | 5394 | 3-methyl-3- | −7.938 | 2 hydrogen |
| | | | oxoimidazo[5,1-d] [1,2,3,5] | | interactions |
| | | | tetrazine-8-carboxamide | | with Cys192 |
| | | | | | and Asp1040 |
| 2 | MTIC | 54422836 | 3-methyl-(triazen-1-yl) | −6.829 | 2 hydrogen |
| | | | imidazole-4-carboxamide | | interactions |
| | | | | | with Cys192 |
| | | | | | and Asp1040 |
| | | | | | π-π interaction |
| | | | | | with Phe1090 |
| 3 | AIC | 9679 | 4-amino-1H-imidazole-5- | −5.736 | 2 hydrogen |
| | | | carboxamide | | interactions |
| | | | | | with Val907 |
| | | | | | and Glu878 |
| | | | | | π-π interaction |
| | | | | | with Lys816 |
| 4 | M | 14219 | 3-(diaminomethylidene)- | −4.194 | 3 hydrogen |
| | | | 1,1-dimethylguanidine; | | interactions |
| | | | hydrochloride | | with Asp1040, |
| | | | | | Arg1021, and |
| | | | | | Asp807 |
| | | | | | π-π interaction |
| | | | | | with His1020 |
| 5 | GUA | 8859 | di-amino methylidene- | −3.855 | 2 hydrogen |
| | | | eurea | | interactions |
| | | | | | with Glu878 |
| | | | | | and Ile1038 |
| 6 | E | 65064 | [(2R,3R)-5,7-dihydroxy-2- | −3.397 | 2 hydrogen |
| | | | (3,4,5-trihydroxyphenyl)- | | interactions |
| | | | 3,4-dihydro-2H-chromen- | | with Glu878 |
| | | | 3-yl] 3,4,5- | | and Asp1040 |
| | | | trihydroxybenzoate | | |

### Example 10—Combination of MET and EGCG Enhance the Anti-Proliferative Potencies of Sorafenib (SOR) and Cisplatin (CPL) in Hepatocellular Carcinoma and Ovarian Cancer Cells

Cell lines and culture conditions. A human hepatocellular carcinoma (HepG2) and ovarian cancer (SKOV3) cells were obtained from National Centre for Cell Science (NCCS), Pune, India. The cells HepG2 and SKOV3 were sub-cultured in Eagle's Minimum Essential Medium (EMEM) and McCoy's 5a supplemented with 10% FBS at 5% CO₂ and 37° C. At 85% confluence, the cells were harvested using 0.25% trypsin and seeded in 25 cm² flasks, 96 well, and 6-well plates, according to the experiment being performed. The cells were allowed to attach 70% to the surface prior to treatment. A stock solution of all the drugs (10 mg/ml) was made in distilled H₂O and diluted to the required concentrations. Suspensions were aspirated 10 times before treatment. Cells treated with vehicle control were used as a control.

Cell viability assay. HepG2 and SKOV3 cells (5×10³ cells/ml) were seeded in 96-well plates and exposed to different individual-drug treated groups with varying concentrations (0, 10, 40, 80, 120, and 160 μM) for a period of 24 hours. The individual-treated groups include Group I: SOR/CPL-treated HepG2 and SKOV3 cells respectively; Group II: MET-treated HepG2 and SKOV3 cells; Group III: EGCG-treated HepG2 and SKOV3 cells and Group IV: SOR+MET+EGCG and CPL+MET+EGCG treated HepG2 and SKOV3 cells respectively.

Results: The anti-proliferative effect of SOR, MET, and EGCG and CPL, MET, and EGCG, alone and in combination, on HepG2 and SKOV3 cells respectively was examined according to the methods described above. As shown in (FIG. 18), exposure of HepG2 cells to SOR, MET, and EGCG both individually and in combination resulted in a reduction in cell viability in a dose-dependent fashion. As seen in the FIG. 18 the triple-drug combination most significantly reduced cell viability as compared to SOR alone as well as MET and EGCG alone. A similar trend was observed in ovarian cancer SKOV3 cell line, wherein the triple combination of CPL+MET+EGCG showed significant antiproliferative activity as compared to the individual treatment with CPL, MET and EGCG (FIG. 19).

### Example 11—Combination of Metformin (MET) and Epigallocatechin Gallate (EGCG) Enhance the Anti-Proliferative Potencies of 5 Fluorouracil (5FU) in Pancreatic Cancer and Colon Cancer Cells

Cell lines and culture conditions: A human pancreatic cancer cell line PANC1 and human colon cancer cell line HCT116 were obtained from National Centre for Cell Science (NCCS), Pune, India. The PANC1 cells were sub-cultured in Dulbecco's Modified Eagle's Medium (DMEM) supplemented with 10% FBS at 5% C02 and 37° C. The HCT116 cells were sub-cultured in McCoy's 5A media supplemented with 10% FBS at 5% CO₂ and 37° C. At 85% confluence, the cells were harvested using 0.25% trypsin and seeded in 96 well plate. The cells were allowed to attach 70% to the surface prior to treatment. 5-Fluorouracil (United Biotech Limited, India), metformin (MET) and epigallocatechin gallate (EGCG) stock solution (10 mg/ml) was made in sterile water and diluted to the required concentrations. Suspensions were aspirated 10 times before treatment. Cells treated with vehicle control were used as a control.

Cell viability assay: PANC1 and HCT116 cells (5×10′ cells/ml) were seeded in 96-well plates and exposed to different individual-drug treated groups with varying concentrations (0, 20, 40, 60, 80, 100, 120, 140, 160, 180 and 200 μM) for a period of 24 hours. The individual-treated groups include Group I: 5 Fluorouracil (5FU)-treated PANC1 and HCT116 cells; Group II: MET-treated PANC1 and HCT116 cells; Group III: EGCG-treated PANC1 and HCT116 cells and Group IV: 5FU+MET+EGCGtreated PANC1 and HCT116 cells.

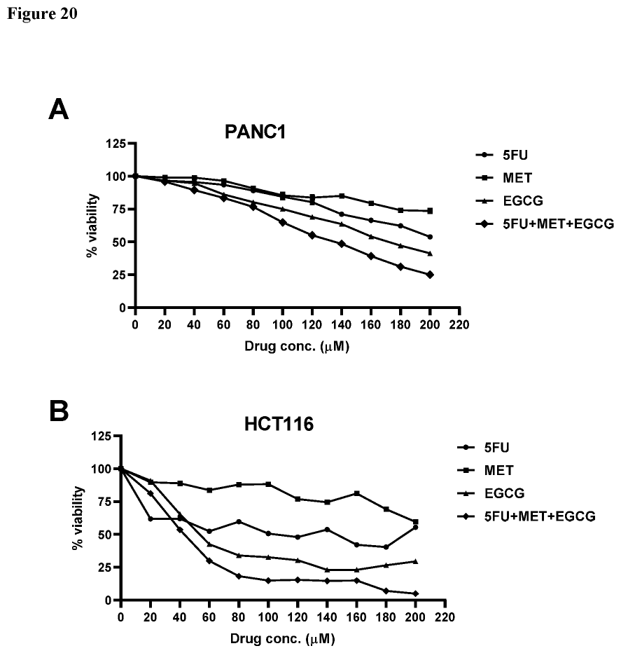

Results: The anti-proliferative effect of 5FU, MET, and EGCG alone and in combination on PANC1 and HCT116 cells was examined according to the methods described above. As shown in FIG. 20A, exposure of pancreatic cancer cells PANC1 to 5FU, MET, and EGCG both individually and in combination resulted in a reduction in cell viability in a dose-dependent fashion. As seen in the FIG. 20A the triple-drug combination most significantly reduced cell viability as compared to 5FU alone as well as MET and EGCG alone. A similar trend was observed in colon cancer HCT116 cell line, wherein the triple combination of 5FU+MET+EGCG showed significant antiproliferative activity as compared to the individual treatment with 5FU, MET and EGCG (FIG. 20B).

### Example 12—Combination of Metformin (MET) and Epigallocatechin Gallate (EGCG) Enhance the Anti-Proliferative Potencies of Doxorubicin (DOX) in Breast Cancer and Bone Cancer Cells

Cell lines and culture conditions: A human breast cancer cell line MDA-MB-453 and human bone cancer cell line SaoS2 were obtained from National Centre for Cell Science (NCCS), Pune, India. The MDA-MB-453 cells were sub-cultured in Dulbecco's Modified Eagle's Medium (DMEM) supplemented with 10% FBS at 5% CO₂ and 37° C. The SaoS2 cells were sub-cultured in McCoy's 5A media supplemented with 10% FBS at 5% CO₂ and 37° C. At 85% confluence, the cells were harvested using 0.25% trypsin and seeded in 96 well plate. The cells were allowed to attach 70% to the surface prior to treatment. Doxorubicin (DOX), metformin (MET) and epigallocatechin gallate (EGCG) stock solution (10 mg/ml) was made in sterile water and diluted to the required concentrations. Suspensions were aspirated 10 times before treatment. Cells treated with vehicle control were used as a control.

Cell viability assay: MDA-MB-453 and SaoS2 cells (5×10³ cells/ml) were seeded in 96-well plates and exposed to different individual-drug treated groups with varying concentrations (0, 20, 40, 60, 80, 100, 120, 140, 160, 180 and 200 μM) for a period of 24 hours. The individual-treated groups include Group I: Doxorubicin (DOX)-treated MDA-MB-453 and SaoS2 cells; Group II: MET-treated MDA-MB-453 and SaoS2 cells; Group III: EGCG-treated MDA-MB-453 and SaoS2 cells and Group IV: DOX+MET+EGCG treated cells.

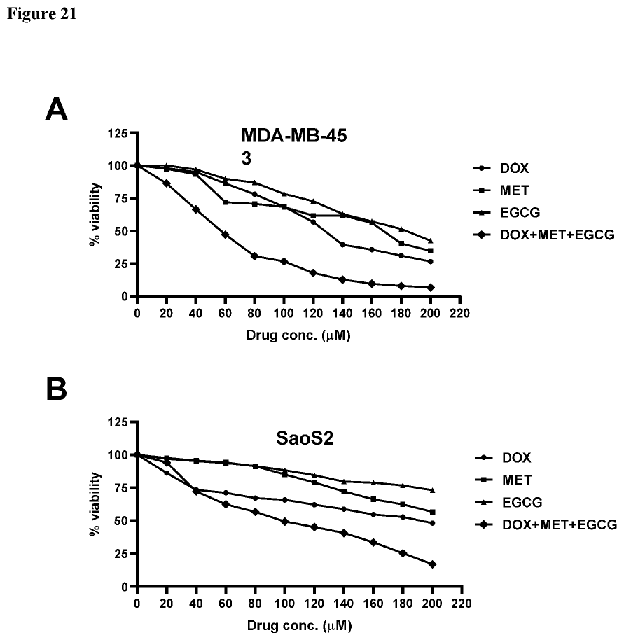

Results: The anti-proliferative effect of DOX, MET, and EGCG alone and in combination on MDA-MB-453 and SaoS2 cells was examined according to the methods described above. As shown in FIG. 21A, exposure of breast cancer cells MDA-MB-453 to DOX, MET, and EGCG both individually and in combination resulted in a reduction in cell viability in a dose-dependent fashion. As seen in the FIG. 21A the triple-drug combination most significantly reduced cell viability as compared to DOX alone as well as MET and EGCG alone. A similar trend was observed in bone cancer SaoS2 cell line, wherein the triple combination of DOX+MET+EGCG showed significant antiproliferative activity as compared to the individual treatment with DOX, MET and EGCG (FIG. 21B).

### Example 13—Anti-Warburg Effect of Temozolomide (T), Metformin (M) and Epigallocatechin Gallate (E) on In Vitro and In Vivo Glioma Model

The key hallmark of glioblastoma aggressiveness and chemoresistance is the altered glucose metabolism. The mechanism is majorly driven by “the Warburg effect” where GBM cells employ aerobic glycolysis regardless of oxygen availability via reprogramming of mitochondrial oxidative phosphorylation. The anti-Warburg effect of triple drug combination studied in this example.

### Cell Line and Culture Conditions

Human GB cell line, U-87 MG and rat glioma cell line C6 were obtained from National Centre for Cell Science (NCCS), Pune, India. The cells were sub-cultured in Dulbecco's Modified Eagle's Medium (DMEM) supplemented with 10% FBS at 5% CO₂ and 37° C. On reaching 85% confluence, the cells were harvested using 0.25% trypsin and used accordingly. For the treatment, the stock solution (10 mM) of each drug (Temozolomide (T), metformin (M), epigallocatechin gallate (E)) was made with sterile water and were further diluted using serum-free medium to arrive at the required concentrations. Suspensions were aspirated 10 times before treatment. Cells treated with vehicle control were used as a control.

### Orthotropic Xenograft Glioma Model:

In vivo glioma model generation, study design, group assignment, drug dose and other relevant information are disclosed in the Example-5. In brief, Wistar rats were anesthetized and using a stereotaxic head frame 1×10⁶ C6 glioma cells was injected at right striatum. The rats were monitored daily for signs of distress and death. Treatment with drugs was commenced 20 days after orthotopic implantation of glioma cells. The same study samples in Example-5 used to analyse the anti-Warburg effect.

### Primary Culture of Astrocytes

Primary astrocytes were collected from the isolated brain tissues as described by Schildge, et aL. (J. Vis. Exp. (71), e50079). In brief, the brain tissues of all experimental groups were isolated, immediately after decapitation. The minced tissue was dissociated using 0.05% trypsin at 37° C. for 30 min. To isolate individual astrocytes, the cell suspension was passed through 40 m strainer. At last, astrocytes were seeded at a density of 1.5×10⁵ cells/cm² in 90% DMEM containing 10% FBS, and 4.5 g/L glucose. The astrocytes were cultured at 37° C. with 95% air and 5% CO₂.

### Glucose Uptake Assay

Glucose uptake assay was performed using Glucose Uptake-Glo kit (Promega, USA). The assay was performed according to the manufacturer's protocol, for both the cell lines and astrocytes isolated from the rat brain tissue.

Quantitative Real-Time Polymerase Chain Reaction (qRT-PCR)

The qPCR analyses were performed to measure the expression levels of genes associated with anti-Warburg effect. The total RNA was extracted from the cell lysates and brain tissues of all the experimental groups using TRIZOL reagent (Takara Bio, Shiga, Japan). Quantitative RT-PCR was carried out using a CFX96 Real Time PCR Detection System (Bio-Rad, California, USA) and TB Green Premix Ex Taq I (Takara Bio, Shiga, Japan) to detect messenger ribonucleic acid (mRNA).

The specific PCR primer sequences are listed in Table 6 below. Independent experiments were conducted in triplicate. The relative changes in gene expression were calculated with the 2^{-ΔΔCt} method, where ΔΔCt=Sample (Ct_{target gene}−Ct_{β-actin})−Calibrator (Ct_{target gene}−Ct_{β-actin}).

TABLE 6
qPCR primer sequences used to measure
the expression levels of genes
associated with anti-Warburg effect.

| | Gene | Forward (5′-3′) | Reverse (5′-3′) |
|---|---|---|---|
| Human | GLUT1 | CGGATGAAGAGAGGCATGTT | CAATCACACCACAAGCC |
| | | (SEQ ID NO: 25) | AAG |
| | | | (SEQ ID NO: 26) |
| | GLUT4 | GCTCTTCTGGACAAGTACAA | TTACACGGATGA |
| | | TGCTG | ACGCTAAGCTTC |
| | | (SEQ ID NO: 27) | (SEQ ID NO: 28) |
| | PKM2 | GGCAGCTATGTGAGAGCC | CTGACGTCCACCCTGAC |
| | | (SEQ ID NO: 29) | (SEQ ID NO: 30) |
| | LDHV | CAAATCCCACCTTGAACACA | CGACTGACTAATGGCAG |
| | | (SEQ ID NO: 31) | CAG |
| | | | (SEQ ID NO: 32) |
| | MCT1 | CAAATCCCACCTTGAACACA | CGACTGACTAATGGCAG |
| | | (SEQ ID NO: 33) | CAG |
| | | | (SEQ ID NO: 34) |
| | MCT4 | CAAATCCCACCTTGAACACA | CGACTGACTAATGGCAG |
| | | (SEQ ID NO: 35) | CAG |
| | | | (SEQ ID NO: 36) |
| Rat | GLUT1 | CAATGGCGGCGGTCCTATAA | GGAGAATGGACGTGCTG |
| | | (SEQ ID NO: 37) | TGA |
| | | | (SEQ ID NO: 38) |
| | GLUT4 | ATCGCTCTGTGGATGACTGA | AGAGACAGCCAGGAGAA |
| | | GTAC | ATCAAAC |
| | | (SEQ ID NO: 39) | (SEQ ID NO: 40) |
| | PKM2 | CATGCTGATGTCTCTGGAAT | GATGTGATGCCTCTGCG |
| | | CT | AAG |
| | | (SEQ ID NO: 41) | (SEQ ID NO: 42) |
| | LDHV | AGTTCCCGGGTGCTGTCTAT | GCCATGGTCTTTCTGCT |
| | | (SEQ ID NO: 43) | CAC |
| | | | (SEQ ID NO: 44) |
| | MCT1 | AGTTCCCGGGTGCTGTCTAT | GCCATGGTCTTTCTGCT |
| | | (SEQ ID NO: 45) | CAC |
| | | | (SEQ ID NO: 46) |
| | MCT4 | AGAGCTACGAGCTGCCTGAC | AGCACTGTGTTGGCGTA |
| | | (SEQ ID NO: 47) | CAG |
| | | | (SEQ ID NO: 48) |

### Enzyme-Linked Immune Sorbent Assay (ELISA)

ELISA for U-87 MG, C6 cells and tumor-induced brain tissues was performed to quantify the protein levels GLUT-1 (Human: MBS8800801; Rat: MBS8805788), PKM2 (Human: MBS8800515; Rat: MBS8800516), LDHV (Human: MBS8801237; Rat: MBS8804669) and MCT-1 (Human: MBS8803496; Rat: MBS8806122) (My BioSource, California, USA). The concentration levels of the above-mentioned proteins were calculated using a standard curve obtained.

### Statistical Analysis

Experiments were performed in triplicates and the quantitative values are expressed as the mean standard deviation (SD). All the experimental data were subjected to two-way ANOVA using GraphPad Prism (GraphPad Software Inc., California, USA).

### Results

### The Triple-Drug Combination Hindered the Uptake of Glucose

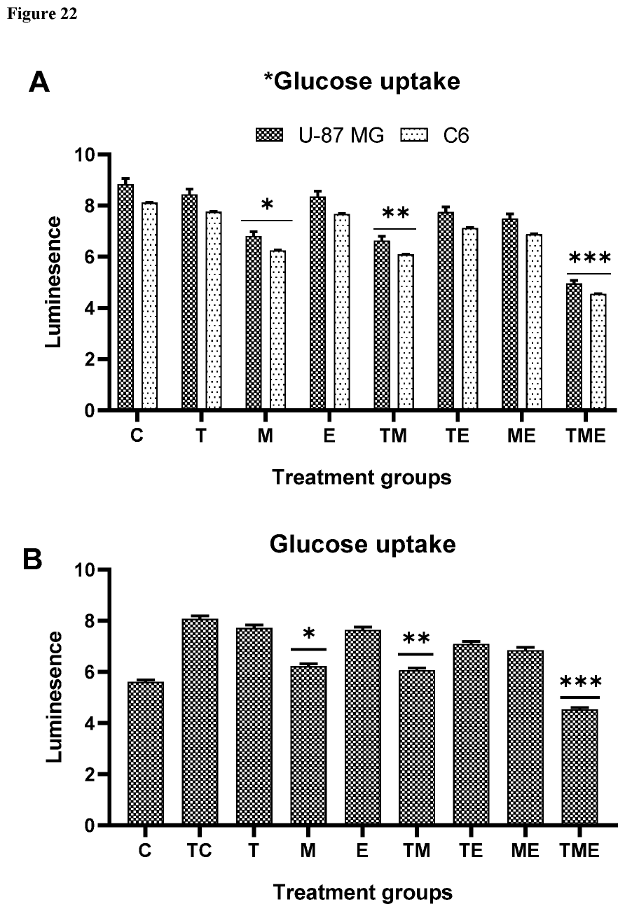

The efficacy of the chosen drugs on glucose uptake levels in U-87 MG and C6 glioma cells as well as in rat primary brain tissue to determine if the chosen three drugs either individually or as a combination could contribute to reduction in the uptake of glucose levels. Interestingly, we observed that the triple-drug treatment (TME) showed a significant reduction in glucose uptake both in vitro (FIG. 22A; p<0.001) and in vivo (FIG. 22B; p<0.001). Further, the dual treatment of TM and individual treatment with M had also reduced glucose uptake significantly in both the cell lines and in the brain tissues of GB-bearing xenograft rats (p<0.01 & p<0.05, respectively; FIG. 22).

### The Anti-Warburg Effect of the Triple-Drug Combination

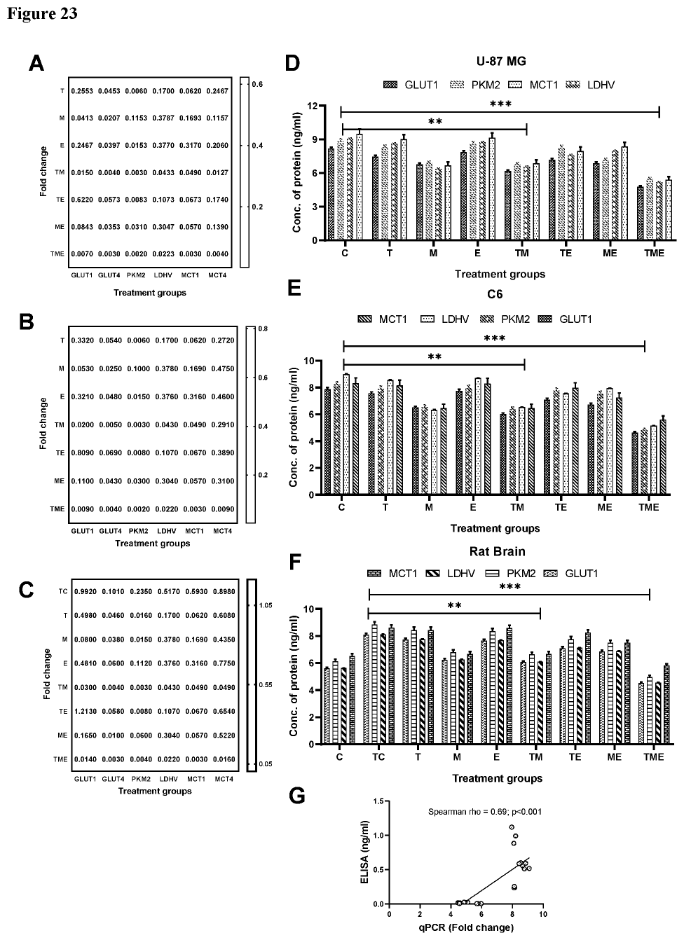

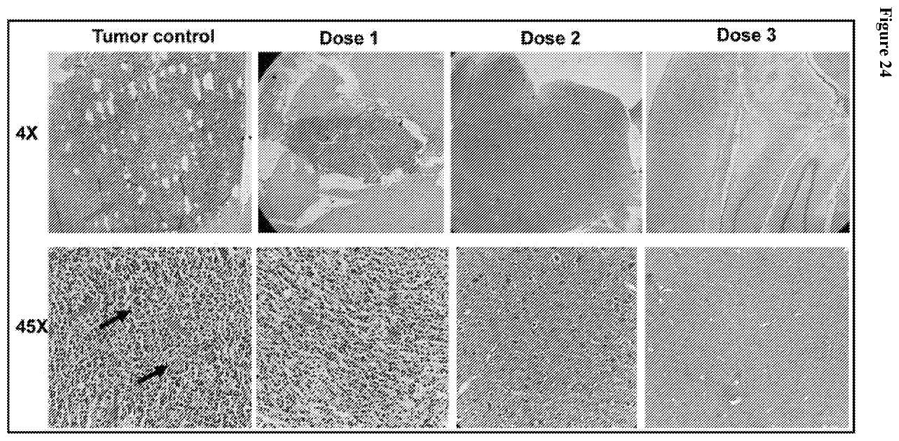

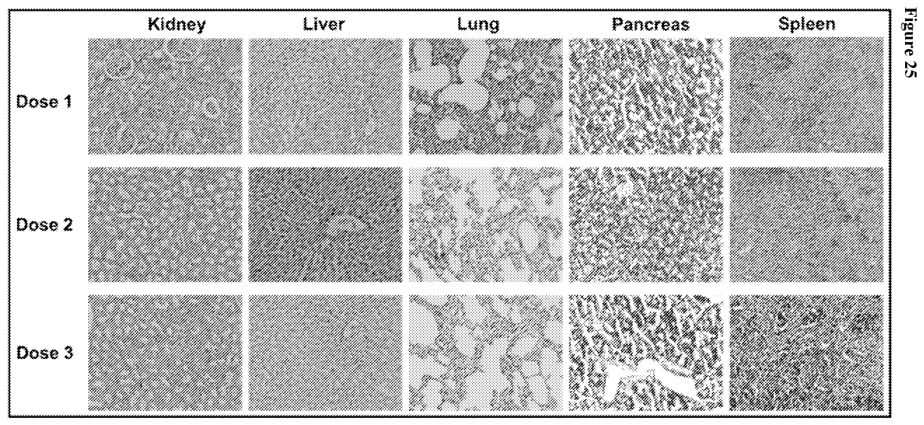

Key markers of Warburg effect namely, GLUT1, GLUT4, PKM2, LDHV, MCT1 and MCT4 were measured at RNA expression levels through qRT-PCR. Likewise, protein expression of GLUT1, PKM2, LDHV and MCT1 were quantified by ELISA. Interestingly, the expression studies highlighted that the gene expression and protein levels of theses markers were highly upregulated in the non-treated U-87 MG and C6 glioma cells and Tumor control (TC) group, whereas, the triple drug combination (TME) had significantly reduced the levels of all these markers in both the glioma cell lines as well as in GB-tissue bearing xenografts (p<0.001; FIG. 23A-F). Also, the dual-drug treatment of TM too had significantly reduced the levels of these markers in both glioma cells and xenografts which can be attributed to the fact that MET is known to reduce glucose uptake and rate of glucose metabolism (p<0.01; FIG. 23A-F). Furthermore, the data generated by qRT-PCR and ELISA showed a significant corelation between the two assays (Spearman rho 0.69; p<0.001; FIG. 23G).

### Example 14—Metabolites of Triple Combination Drugs Temozolomide (T), Metformin (M) and Epigallocatechin Gallate (E) Transport Across the Blood Brain Barrier

### Orthotropic Xenograft Glioma Model:

To explore if the active metabolites of T, M, and E transport across the blood brain barrier, an in vivo glioma model generated according to the information are disclosed in the Example-5. T, M & E (Sigma Aldrich, St. Louis, USA) were dissolved in sterile water and administered intra peritoneally for 7 days (from day 21 to day 27; where the day of tumor induction is considered day 0) to a rat with the dose specified in Table-4 (TME: 50+80+150 mg/kg body weight). After 7 days of treatment (on day 28), blood (plasma) was collected and the rat was euthanized, immediately the brain tissue was collected and stored at −80° C. The brain tissue was then processed accordingly to measure active metabolites through LC/MS.

### Sample Preparation

To 100 μl of plasma or brain (0.5 g crushed in 0.5 mL of sterile water), 20 μl of the working internal standard solution was added. The samples were deproteinised with acetonitrile and the supernatant (100 μl) was injected into the chromatographic system. For the brain, as described above, ciprofloxacin solution (20 μl) was added to 100 μl of crushed brain. Then, repeating the plasma sample process, 20 μl of the solution was injected into the chromatographic system. The chromatographic conditions used are given in Table 7.

TABLE 7
Chromatographic conditions and mass-spectrometry details of metabolites

| | | Metabolites | Mass/charge |
|---|---|---|---|
| Drug | Chromatographic conditions | analysed | value (m/z) |
| Temozolomide | Ammonium formate (pH 3.0; | Temozolomide | 194.151 |
| | 10 mM) (solvent A) and acetonitrile | (T) | |
| | (solvent B). | 3-methyl-(triazen- | 168.16 |
| | Gradient elution with 5% solvent B, | 1-yl) imidazole-4- | |
| | increased with a linear increment to | carboxamide | |
| | 95% | (MTIC) | |
| | | 5-amino- | 126.12 |
| | | imidazole-4- | |
| | | carboxamide | |
| | | (AIC) | |
| Metformin | The compounds have to be eluted | Metformin (M) | 129.16 |
| | with a mobile phase of 10% | Guanylurea | 102.10 |
| | acetonitrile in water with 40 mM | (GUA) | |
| | ammonium carbonate (pH 8) at a | | |
| | flow rate of 0.2 mL/min. | | |
| Epigallocatechin | The samples were diluted in | Epigallocatechin | 458.37 |
| Gallate | Acetonitrile/water (80:20 v/v) The | Gallate (E) | |
| | organic phase is collected and | | |
| | dried at 35° C. and reconstituted in | | |
| | the mobile phase (Acetonitrile/ | | |
| | water- 80:20 v/v). | | |

### Results: Triple Combination Drugs Successfully Transport Across the Blood Brain Barrier

From the LC/MS analysis it was evident that all the three drugs (T, M & E) reached the circulatory system as measured in rat blood plasma and crossed the blood brain barrier (BBB) either in their native form or as in the form of their active metabolites (Table-8 and Table-9). This effect was observed both when the drugs were administered individually and in combination of T, M & E (Table-8 and Table-9). The drugs T and M were found to have crossed BBB in the form of their active metabolites AIC & GUA respectively while E was found to have crossed the BBB in its native form.

TABLE 8
LC-MS data for individually treated T, M and E. The
calculated mass and observed mass displayed in the
table. NA correspond to ‘Not Applicable’ where
respective drug or their active metabolites are not detected.

| Sl. | | Actual | Detected mass in LC-MS | |
|---|---|---|---|---|
| no. | Drug/metabolite | mass | Plasma | Brain |
| 1. | Temozolomide (T) | 194.151 | NA | NA |
| 2. | 3-methyl-(triazen-1-yl) | 168.16 | NA | NA |
| | imidazole-4-carboxamide | | | |
| | (MTIC) | | | |
| 3. | 5-amino-imidazole-4- | 126.12 | 127.02 | 123.09 |
| | carboxamide (AIC) | | | |
| 4. | Metformin (M) | 129.16 | 129.06 | 123.05 |
| 5. | Guanylurea (GUA) | 102.10 | NA | NA |
| 6. | Epigallocatechin Gallate (E) | 458.37 | NA | 466.11 |

TABLE 9
LC-MS data for Triple combination drugs (T, M, and E).
The calculated mass and observed mass displayed in the
table. NA correspond to ‘Not Applicable’ where
respective drug or their active metabolites are not detected.

| Sl. | | Actual | Detected mass in LC-MS | |
|---|---|---|---|---|
| no. | Drug/metabolite | mass | Plasma | Brain |
| 1. | Temozolomide (T) | 194.151 | 198.93 | NA |
| 2. | 3-methyl-(triazen-1-yl) | 168.16 | NA | NA |
| | imidazole-4-carboxamide | | | |
| | (MTIC) | | | |
| 3. | 5-amino-imidazole-4- | 126.12 | 127.02 | 123.05 |
| | carboxamide (AIC) | | | |
| 4. | Metformin (M) | 129.16 | 130.16 | 130.16 |
| 5. | Guanylurea (GUA) | 102.10 | NA | NA |
| 6. | Epigallocatechin Gallate (E) | 458.37 | 452.91 | 456.11 |

### Example 15—In Vivo Glioma Model: Toxicity and Dose Response Effect of Orally Administered Temozolomide (T), Metformin (M) and Epigallocatechin Gallate (E) on Tumor Reduction and Angiogenesis Blockade

### Orthotropic Xenograft Glioma Model:

To assess the dose specific effect of T, M, and E in reducing the glioma tumor burden and angiogenesis blockade, an in vivo glioma model generated according to the information are disclosed in the Example-5. In this independent study, a total of 15 animals were included. Tumor was induced as explained previously (Example-5) and after 20 days of incubation the animals were randomly segregated into 4 different groups as shown in the Table 10.

TABLE 10
Study design, animal numbers and dose details
of each drug in triple drugs combination

Rat dose

| | Tumor | Dose 1 | Dose 2 | Dose 3 |
|---|---|---|---|---|
| | control | (mg/Kg) | (mg/Kg) | (mg/Kg) |
| Drug | (n = 3) | (n = 4) | (n = 4) | (n = 4) |
| T | NA | 10 | 30 | 50 |
| M | NA | 15 | 50 | 80 |
| E | NA | 25 | 100 | 150 |

### Drug Dosage

All the drugs (T, M & E; Sigma Aldrich, St. Louis, USA) were freshly prepared as aqueous solutions in sterile water and were administered orally via an oral gavage once a day for a period of 7 consecutive days, the respective concentrations are as shown in Table 10.

### Haematoxylin and Eosin Staining

After treatment for 7 days, the rats were euthanized on Day-8, and the brain tissues of the experimental groups were collected and fixed in 4% PBS-buffered paraformaldehyde followed by embedding in paraffin. In order to perform the haematoxylin and eosin (H & E) staining, paraffin blocks were sectioned by 5 μm thickness. Slides were then stained with H & E and were later observed under 4× and 45× magnification in a compound microscope (Auxiovert, Carl Zeiss, USA) to take digital photographs. Only two animal tissues were processed for the H&E staining and representative figures were displayed, and other samples were kept for future analyses.

### Drug Toxicity Analysis

To assess the drug-induced toxicity in major vital organs, the organs namely liver, lungs, spleen, kidney and pancreas were collected after rats were euthanized and processed for histopathological analysis using H & E staining.

### Results

### Triple Drug Combination Induces Dose Specific Response on Tumor Reduction and Angiogenesis Blockade

Histological analyses by H & E staining depicted a considerable decrease in tumor cells in the sections obtained from all the 3 doses of the triple-drug combination (TME) when compared with Tumor control. The tumor-control (TC) rats depicted intense cellularity and extensive haemorrhage in blood vessels, as similar to the WHO Grade II glioma (FIG. 24, Tumor control). In contrast, the cellularity and haemorrhage were drastically reduced in the rats treated with triple drug combination (TME) in a dose dependent manner (FIG. 24). While Dose 1 & 2 treatment portrayed a slight reduction in the whorled structures and reduced cell density but profound tumor clearance was observed with Dose-3 (FIG. 24).

### Triple Drug Combination Demonstrates a Good Safety Profile

Analysis of the vital organ tissue sections after H & E staining showed that the Dose-2 (mid) and Dose-3 (high) group were exhibited similar safety profile to the Dose-1 (low) group. Some level of lesions were noticed for spleen in Dose-3 group. However, no significant damage was observed in either of the three treatments. From these results it is evident that TME combination at all doses including a higher dose (Dose-3) is safer with minimal or no toxic side effects to the animal (FIG. 25).

### Example 16—In Vivo Glioma Model: Dose Response Effect of Orally Administered Temozolomide (T), Metformin (M) and Epigallocatechin Gallate (E) on Animal Survival

### Orthotropic Xenograft Glioma Model:

To assess the dose specific effect of T, M, and E on animal survival, an in vivo glioma model generated according to the information are disclosed in the Example-5. In this independent study, another 15 animals were included. Tumor was induced in all the 15 animals as explained previously (Example-5) and after 20 days of incubation the animals were randomly segregated into 4 different groups as shown in the Table 11.

TABLE 11
Study design, animal numbers and dose details of triple drugs

Rat dose

| | Tumor | Dose-1 | Dose-2 | Dose-3 |
|---|---|---|---|---|
| | control | (mg/Kg) | (mg/Kg) | (mg/Kg) |
| Drug | (n = 3) | (n = 4) | (n = 4) | (n = 4) |
| T | NA | 10 | 30 | 50 |
| M | NA | 15 | 50 | 80 |
| E | NA | 25 | 100 | 150 |

### Drug Dosage

All the drugs (T, M & E; Sigma Aldrich, St. Louis, USA) were freshly prepared as aqueous solutions in sterile water and were administered orally via an oral gavage once a day for a period of 7 consecutive days, the respective concentrations are as shown in Table 11.

### Survival Study

Survival study was carried out on a set of 15 rats that were implanted with C6 rat glioma cells and treated with the respective drug dosage as mentioned previously in Table 11. After the treatment period, the animals were continuously monitored for their survival rate for a period of 23 weeks. In the survival analysis, the death of a rat in each group of treatment was taken as a break point and a survival graph was plotted using GraphPad Prism 8 (San Diego, USA).

### Results: Triple Drug Combination Induces Dose Specific Improvement in Animal Survival

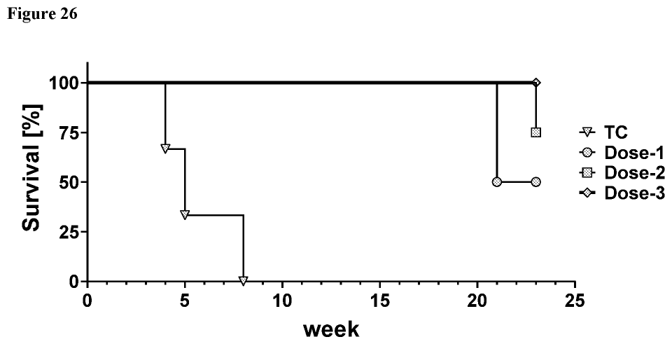

At the midway of the study, the overall survival was interpolated using Kaplan-Meier Survival graph. As shown in FIG. 26, treatment of the rats with the triple-drug combination (T, M & E) at dose-3 had significantly improved tumor suppression and prolonged the duration of survival in 100% of the treated animals (>23 weeks), whereas all the animals in tumor control group died at week-8. The median survival rate of the Dose-3 treated animals was significantly higher than the tumor-control group (>23 weeks vs. 5.6 weeks; p<0.001). On the other hand, among the other two treatment modalities of Dose-1 & Dose 2, Dose-2 showed a marginally better median survival rate of 22.75 weeks relative to Dose-1 group with 21 weeks. Altogether, the survival study demonstrated a dose specific improvement in accord with the histology data of Example-15.

1. A pharmaceutical composition comprising metformin and epigallocatechin gallate.

2. The pharmaceutical composition of claim 1, further comprising a chemotherapeutic agent.

3. The composition of claim 2, wherein the chemotherapeutic agent is temozolomide (TMZ), sorafenib, cisplatin, topotecan, pegylated liposomal doxorubicin (PLD), thiotepa, cyclosphosphamide, busulfan, improsulfan, piposulfan, aziridines, benzodopa, carboquone, meturedopa, uredopa, ethylenimines, altretamine, triethylenemelamine, trietylenephosphoramide, triethiylenethiophosphoramide, trimethylolomelamine, acetogenins, bullatacin, bullatacinone, a camptothecin, bryostatin; callystatin; CC-1065, adozelesin, carzelesin, bizelesin, cryptophycin 1, cryptophycin 8, dolastatin; duocarmycin, KW-2189, CB1-TM1, eleutherobin, pancratistatin, a sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, nitrosureas, carmustine, chlorozotocin, fotemustine, lomustine, nimustine, ranimnustine, enediyne antibiotics, calicheamicins, dynemicin A; bisphosphonates, clodronate, an esperamicin, neocarzinostatin, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin, deoxydoxorubicin, epirubicin, esorubicin, idarubicin, marcellomycin, mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; methotrexate, 5-fluorouracil (5-FU); folic acid analogues, denopterin, pteropterin, trimetrexate; purine analogs, fludarabine, 6-mercaptopurine, thiamiprine, thioguanine, pyrimidine analogs, ancitabine, azacytidine, 6-azauridine, carmofur, cytarabine, dideoxyuridine, doxifluridine, enocitabine, floxuridine; androgens, calusterone, dromostanolone propionate, epitiostanol, mepitiostane, testolactone; anti-adrenals, aminoglutethimide, mitotane, trilostane; folic acid replenishers, frolinic acid, aceglatone, aldophosphamide glycoside, aminolevulinic acid, eniluracil, amsacrine, bestrabucil, bisantrene, edatrexate, defofamine, demecolcine, diaziquone, elformithine, elliptinium acetate, an epothilone, etoglucid, gallium nitrate, hydroxyurea, lentinan, lonidainine, maytansinoids, maytansine, ansamitocins, mitoguazone, mitoxantrone, mopidanmol; nitraerine, pentostatin, phenamet, pirarubicin, losoxantrone, podophyllinic acid, 2-ethylhydrazide, procarbazine, razoxane, rhizoxin, sizofuran, spirogermanium, tenuazonic acid, triaziquone; 2,2′,2″-trichlorotriethylamine, trichothecenes, T-2 toxin, verracurin A, roridin A, anguidine, urethan, vindesine, dacarbazine, mannomustine, mitobronitol, mitolactol, pipobroman, gacytosine, arabinoside (“Ara-C”), cyclophosphamide, thiotepa, taxoids, paclitaxel, an albumin-engineered nanoparticle formulation of paclitaxel, docetaxel, chlorambucil, gemcitabine, 6-thioguanine, mercaptopurine, methotrexate, platinum analogs, oxaliplatin, carboplatin, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluoromethylornithine (DFMO), retinoic acid, capecitabine, combretastatin, leucovorin (LV), oxaliplatin, lapatinib, inhibitors of PKC-alpha, Raf, H-Ras, EGFR, erlotinib, VEGF inhibitors and pharmaceutically acceptable salts, acids or derivatives of any of the above.

4. The composition of claim 2, wherein the chemotherapeutic agent is selected from the group consisting of temozolomide, sorafenib, cisplatin, 5-fluorouracil and doxorubicin.

5. The composition of claim 3, wherein the chemotherapeutic agent is temozolomide.

6. The composition of claim 3, wherein the chemotherapeutic agent is sorafenib.

7. The composition of claim 3, wherein the chemotherapeutic agent is cisplatin.

8. The composition of claim 3, wherein the chemotherapeutic agent is 5-fluorouacil.

9. The composition of claim 3, wherein the chemotherapeutic agent is doxorubicin.

10. The composition of any of claims 1-9, wherein the composition is formulated for parenteral, intrathecal, topical, oral, sublingual, subcutaneous, intraperitoneal, intrapulmonary, nasal, inhalation, or intralesional/intratumoral administration.

11. The composition of any of claims 1-10, wherein the composition is in the form of a tablet, pill, capsule, sachet, effervescent, dragee, lozenge, cream, or a solution suitable for injection.

12. The composition of any of claims 2-11, wherein the amount of each of the chemotherapeutic agent, metformin, and epigallocatechin gallate is about 0.001 mg to about 500 mg, preferably about 0.01 mg/kg to about 500 mg/kg.

13. A kit comprising one or more compositions comprising metformin and epigallocatechin gallate.

14. The kit of claim 13, wherein the kit further comprises a composition comprising a chemotherapeutic agent.

15. The kit of claim 14, wherein the chemotherapeutic agent is TMZ, sorafenib, cisplatin, topotecan, pegylated liposomal doxorubicin (PLD), thiotepa, cyclosphosphamide, busulfan, improsulfan, piposulfan, aziridines, benzodopa, carboquone, meturedopa, uredopa, ethylenimines, altretamine, triethylenemelamine, trietylenephosphoramide, triethiylenethiophosphoramide, trimethylolomelamine, acetogenins, bullatacin, bullatacinone, a camptothecin, bryostatin; callystatin; CC-1065, adozelesin, carzelesin, bizelesin, cryptophycin 1, cryptophycin 8, dolastatin; duocarmycin, KW-2189, CB1-TM1, eleutherobin, pancratistatin, a sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, nitrosureas, carmustine, chlorozotocin, fotemustine, lomustine, nimustine, ranimnustine, enediyne antibiotics, calicheamicins, dynemicin A; bisphosphonates, clodronate, an esperamicin, neocarzinostatin, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin, deoxydoxorubicin, epirubicin, esorubicin, idarubicin, marcellomycin, mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; methotrexate, 5-fluorouracil (5-FU); folic acid analogues, denopterin, pteropterin, trimetrexate; purine analogs, fludarabine, 6-mercaptopurine, thiamiprine, thioguanine, pyrimidine analogs, ancitabine, azacytidine, 6-azauridine, carmofur, cytarabine, dideoxyuridine, doxifluridine, enocitabine, floxuridine; androgens, calusterone, dromostanolone propionate, epitiostanol, mepitiostane, testolactone; anti-adrenals, aminoglutethimide, mitotane, trilostane; folic acid replenishers, frolinic acid, aceglatone, aldophosphamide glycoside, aminolevulinic acid, eniluracil, amsacrine, bestrabucil, bisantrene, edatrexate, defofamine, demecolcine, diaziquone, elformithine, elliptinium acetate, an epothilone, etoglucid, gallium nitrate, hydroxyurea, lentinan, lonidainine, maytansinoids, maytansine, ansamitocins, mitoguazone, mitoxantrone, mopidanmol; nitraerine, pentostatin, phenamet, pirarubicin, losoxantrone, podophyllinic acid, 2-ethylhydrazide, procarbazine, razoxane, rhizoxin, sizofuran, spirogermanium, tenuazonic acid, triaziquone; 2,2′,2″-trichlorotriethylamine, trichothecenes, T-2 toxin, verracurin A, roridin A, anguidine, urethan, vindesine, dacarbazine, mannomustine, mitobronitol, mitolactol, pipobroman, gacytosine, arabinoside (“Ara-C”), cyclophosphamide, thiotepa, taxoids, paclitaxel, an albumin-engineered nanoparticle formulation of paclitaxel, docetaxel, chlorambucil, gemcitabine, 6-thioguanine, mercaptopurine, methotrexate, platinum analogs, oxaliplatin, carboplatin, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluoromethylornithine (DFMO), retinoic acid, capecitabine, combretastatin, leucovorin (LV), oxaliplatin, lapatinib, inhibitors of PKC-alpha, Raf, H-Ras, EGFR, erlotinib, VEGF inhibitors and pharmaceutically acceptable salts, acids or derivatives of any of the above.

16. The kit of claim 14, wherein the chemotherapeutic agent is selected from the group consisting of temozolomide, sorafenib, cisplatin, 5-fluorouracil and doxorubicin.

17. The kit of claim 14, wherein the chemotherapeutic agent is TMZ.

18. The kit of claim 14, wherein the chemotherapeutic agent is sorafenib.

19. The kit of claim 14, wherein the chemotherapeutic agent is cisplatin.

20. The kit of claim 14, wherein the chemotherapeutic agent is 5-fluorouracil.

21. The kit of claim 14, wherein the chemotherapeutic agent is doxorubicin.

22. The kit of any of claims 13-21, wherein the one or more compositions is in the form of a tablet, pill, capsule, sachet, effervescent, dragee, lozenge, cream, or a solution suitable for injection.

23. The kit of any of claims 14-22, wherein each of the chemotherapeutic agent, metformin, and epigallocatechin gallate are in a separate composition.

24. The kit of any of claims 14-22, wherein the chemotherapeutic agent, metformin, and epigallocatechin gallate are in the same composition.

25. The kit of any of claims 14-22, wherein the chemotherapeutic agent is in a separate composition, and the metformin and epigallocatechin gallate are in the same composition.

26. The kit of any of claims 14-25, wherein the amount of each of the chemotherapeutic agent, metformin, and epigallocatechin gallate is about 0.001 mg to about 500 mg, preferably about 0.01 mg/kg to about 500 mg/kg.

27. A method for inhibiting growth or promoting apoptosis of a cancer cell comprising contacting the cancer cell with
a) an effective amount of a chemotherapeutic agent;
b) an effective amount of metformin; and
c) an effective amount of epigallocatechin gallate,
thereby inhibiting growth of the cancer cell or promoting apoptosis of the cancer cell.

28. The method of claim 27, wherein the cancer cell is selected from the group consisting of a bone cancer cell, a brain cancer cell, a breast cancer cell, colon cancer cell, a melanoma cell, a pancreatic cancer cell, a hepatocellular carcinoma cell, an ovarian cancer cell, a head and neck cancer cell, a lung cancer cell, a renal carcinoma cell, a lymphoma cell, a leukemia cell, a sarcoma, and a carcinoma.

29. The method of claim 27, wherein the cancer cell is selected from the group consisting of a brain cancer cell, a breast cancer cell, colon cancer cell, a melanoma cell, a pancreatic cancer cell, a hepatocellular carcinoma cell, an ovarian cancer cell, a head and neck cancer cell, a lung cancer cell, a renal carcinoma cell, a lymphoma cell, a leukemia cell, a sarcoma, and a carcinoma.

30. The method of claim 27, wherein the cancer cell is selected from the group consisting of a bone cancer cell, a brain cancer cell, a breast cancer, a cell colon cancer cell, a pancreatic cancer cell, a hepatocellular carcinoma cell, and a ovarian cancer cell.

31. The method of claim 28 or 29, wherein the cancer cell is a brain cancer cell.

32. The method of claim 31, wherein the brain cancer cell is a glioma cell.

33. The method of claim 28 or 29, wherein the cancer cell is an ovarian cancer cell.

34. The method of claim 28 or 29, wherein the cancer cell is a hepatocellular carcinoma cell.

35. The method of claim 28 or 29, wherein the cancer cell is a pancreatic cancer cell.

36. The method of claim 28, wherein the cancer cell is a bone cancer cell.

37. The method of claim 28 or 29, wherein the cancer cell is a breast cancer cell.

38. The method of claim 28 or 29, wherein the cancer cell is a colon cancer cell.

39. The method of any one of claims 27-38, wherein the cancer cell is a human cancer cell.

40. The method of any one of claims 27-39, wherein the effective amount of each of the chemotherapeutic agent, metformin, and epigallocatechin gallate is about 0.01 to about 500 mg per kilogram of body weight.

41. The method of any one of claims 27-40, wherein the chemotherapeutic agent is temozolomide, sorafenib, cisplatin, topotecan, pegylated liposomal doxorubicin (PLD), thiotepa, cyclosphosphamide, busulfan, improsulfan, piposulfan, aziridines, benzodopa, carboquone, meturedopa, uredopa, ethylenimines, altretamine, triethylenemelamine, trietylenephosphoramide, triethiylenethiophosphoramide, trimethylolomelamine, acetogenins, bullatacin, bullatacinone, a camptothecin, bryostatin; callystatin; CC-1065, adozelesin, carzelesin, bizelesin, cryptophycin 1, cryptophycin 8, dolastatin; duocarmycin, KW-2189, CB1-TM1, eleutherobin, pancratistatin, a sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, nitrosureas, carmustine, chlorozotocin, fotemustine, lomustine, nimustine, ranimnustine, enediyne antibiotics, calicheamicins, dynemicin A; bisphosphonates, clodronate, an esperamicin, neocarzinostatin, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin, deoxydoxorubicin, epirubicin, esorubicin, idarubicin, marcellomycin, mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; methotrexate, 5-fluorouracil (5-FU); folic acid analogues, denopterin, pteropterin, trimetrexate; purine analogs, fludarabine, 6-mercaptopurine, thiamiprine, thioguanine, pyrimidine analogs, ancitabine, azacytidine, 6-azauridine, carmofur, cytarabine, dideoxyuridine, doxifluridine, enocitabine, floxuridine; androgens, calusterone, dromostanolone propionate, epitiostanol, mepitiostane, testolactone; anti-adrenals, aminoglutethimide, mitotane, trilostane; folic acid replenishers, frolinic acid, aceglatone, aldophosphamide glycoside, aminolevulinic acid, eniluracil, amsacrine, bestrabucil, bisantrene, edatrexate, defofamine, demecolcine, diaziquone, elformithine, elliptinium acetate, an epothilone, etoglucid, gallium nitrate, hydroxyurea, lentinan, lonidainine, maytansinoids, maytansine, ansamitocins, mitoguazone, mitoxantrone, mopidanmol; nitraerine, pentostatin, phenamet, pirarubicin, losoxantrone, podophyllinic acid, 2-ethylhydrazide, procarbazine, razoxane, rhizoxin, sizofuran, spirogermanium, tenuazonic acid, triaziquone; 2,2′,2″-trichlorotriethylamine, trichothecenes, T-2 toxin, verracurin A, roridin A, anguidine, urethan, vindesine, dacarbazine, mannomustine, mitobronitol, mitolactol, pipobroman, gacytosine, arabinoside (“Ara-C”), cyclophosphamide, thiotepa, taxoids, paclitaxel, an albumin-engineered nanoparticle formulation of paclitaxel, docetaxel, chlorambucil, gemcitabine, 6-thioguanine, mercaptopurine, methotrexate, platinum analogs, oxaliplatin, carboplatin, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluoromethylornithine (DFMO), retinoic acid, capecitabine, combretastatin, leucovorin (LV), oxaliplatin, lapatinib, inhibitors of PKC-alpha, Raf, H-Ras, EGFR, erlotinib, VEGF inhibitors and pharmaceutically acceptable salts, acids or derivatives of any of the above.

42. The method of any one of claims 27-40, wherein the chemotherapeutic agent is selected from the group consisting of temozolomide, sorafenib, cisplatin, 5-fluorouracil and doxorubicin.

43. The method of claim 41, wherein the chemotherapeutic agent is temozolomide.

44. The method of claim 41, wherein the chemotherapeutic agent is sorafenib.

45. The method of claim 41, wherein the chemotherapeutic agent is cisplatin.

46. The method of claim 41, wherein the chemotherapeutic agent is 5-fluorouracil.

47. The method of claim 41, wherein the chemotherapeutic agent is doxorubicin.

48. A method for treatment or amelioration of a cancer comprising administering to a subject in need thereof:
a) a therapeutically effective amount of a chemotherapeutic agent;
b) a therapeutically effective amount of metformin; and
c) a therapeutically effective amount of epigallocatechin gallate;
thereby treating or ameliorating the cancer in the subject.

49. The method of claim 48, wherein the cancer is selected from the group consisting of brain cancer, breast cancer, colon cancer, colorectal cancer, melanoma, pancreatic cancer, hepatocellular carcinoma, ovarian cancer, head and neck cancer, lung cancer, renal cancer.

50. The method of claim 48, wherein the cancer is selected from the group consisting of bone cancer, brain cancer, breast cancer, colon cancer, colorectal cancer, melanoma, pancreatic cancer, hepatocellular carcinoma, ovarian cancer, head and neck cancer, lung cancer, renal cancer, a lymphoma, a leukemia, a sarcoma, and a carcinoma.

51. The method of claim 48, wherein the cancer is selected from the group consisting of bone cancer, brain cancer, breast cancer, colon cancer, pancreatic cancer, hepatocellular carcinoma, and ovarian cancer.

52. The method of claim 48, wherein the cancer is a brain cancer.

53. The method of claim 48, wherein the brain cancer is a glioma.

54. The method of claim 48, wherein the glioma is glioblastoma multiforme.

55. The method of claim 48, wherein the cancer is a hepatocellular carcinoma.

56. The method of claim 48, wherein the cancer is an ovarian cancer.

57. The method of claim 48, wherein the cancer is a pancreatic cancer.

58. The method of claim 48, wherein the cancer is bone cancer.

59. The method of claim 48, wherein the cancer is breast cancer.

60. The method of claim 48, wherein the cancer is colon cancer.

61. The method of any one of claims 48-60, wherein the subject is a human.

62. The method of any one of claims 48-61, wherein the therapeutically effective amount of the chemotherapeutic agent is about 0.1 to about 200 mg/kg body weight, the therapeutically effective amount of metformin is about 0.1 to about 200 mg/kg body weight, and the therapeutically effective amount of epigallocatechin gallate is about 0.5 mg/kg body weight to about 500 mg/kg body weight.

63. The method of any one of claims 48-60, wherein the chemotherapeutic agent, metformin, and epigallocatechin gallate are administered concurrently.

64. The method of any one of claims 48-60, wherein the chemotherapeutic agent, metformin, and epigallocatechin gallate are administered sequentially.

65. The method of any one of claims 48-60, wherein the metformin and epigallocatechin gallate are administered concurrently and the chemotherapeutic agent is administered sequentially.

66. The method of any one of claims 48-65, wherein the chemotherapeutic agent, metformin, and epigallocatechin gallate are administered via parenteral, intrathecal, subcutaneous, oral, nasal, inhalation, or sublingual administration.

67. The method of any one of claims 48-66, further comprising administering an additional anti-cancer therapy selected from radiation therapy, surgery, an additional chemotherapeutic agent, an anti-angiogenic agent, an immunotherapy, an oncolytic virus, and an anti-inflammatory agent.

68. The method of any of claims 48-67, wherein the chemotherapeutic agent is temozolomide, sorafenib, cisplatin, topotecan, pegylated liposomal doxorubicin (PLD), thiotepa, cyclosphosphamide, busulfan, improsulfan, piposulfan, aziridines, benzodopa, carboquone, meturedopa, uredopa, ethylenimines, altretamine, triethylenemelamine, trietylenephosphoramide, triethiylenethiophosphoramide, trimethylolomelamine, acetogenins, bullatacin, bullatacinone, a camptothecin, bryostatin; callystatin; CC-1065, adozelesin, carzelesin, bizelesin, cryptophycin 1, cryptophycin 8, dolastatin; duocarmycin, KW-2189, CB1-TM1, eleutherobin, pancratistatin, a sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, nitrosureas, carmustine, chlorozotocin, fotemustine, lomustine, nimustine, ranimnustine, enediyne antibiotics, calicheamicins, dynemicin A; bisphosphonates, clodronate, an esperamicin, neocarzinostatin, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin, deoxydoxorubicin, epirubicin, esorubicin, idarubicin, marcellomycin, mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; methotrexate, 5-fluorouracil (5-FU); folic acid analogues, denopterin, pteropterin, trimetrexate; purine analogs, fludarabine, 6-mercaptopurine, thiamiprine, thioguanine, pyrimidine analogs, ancitabine, azacytidine, 6-azauridine, carmofur, cytarabine, dideoxyuridine, doxifluridine, enocitabine, floxuridine; androgens, calusterone, dromostanolone propionate, epitiostanol, mepitiostane, testolactone; anti-adrenals, aminoglutethimide, mitotane, trilostane; folic acid replenishers, frolinic acid, aceglatone, aldophosphamide glycoside, aminolevulinic acid, eniluracil, amsacrine, bestrabucil, bisantrene, edatrexate, defofamine, demecolcine, diaziquone, elformithine, elliptinium acetate, an epothilone, etoglucid, gallium nitrate, hydroxyurea, lentinan, lonidainine, maytansinoids, maytansine, ansamitocins, mitoguazone, mitoxantrone, mopidanmol; nitraerine, pentostatin, phenamet, pirarubicin, losoxantrone, podophyllinic acid, 2-ethylhydrazide, procarbazine, razoxane, rhizoxin, sizofuran, spirogermanium, tenuazonic acid, triaziquone; 2,2′,2″-trichlorotriethylamine, trichothecenes, T-2 toxin, verracurin A, roridin A, anguidine, urethan, vindesine, dacarbazine, mannomustine, mitobronitol, mitolactol, pipobroman, gacytosine, arabinoside (“Ara-C”), cyclophosphamide, thiotepa, taxoids, paclitaxel, an albumin-engineered nanoparticle formulation of paclitaxel, docetaxel, chlorambucil, gemcitabine, 6-thioguanine, mercaptopurine, methotrexate, platinum analogs, oxaliplatin, carboplatin, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluoromethylornithine (DFMO), retinoic acid, capecitabine, combretastatin, leucovorin (LV), oxaliplatin, lapatinib, inhibitors of PKC-alpha, Raf, H-Ras, EGFR, erlotinib, VEGF inhibitors and pharmaceutically acceptable salts, acids or derivatives of any of the above.

69. The method of of any of claims 48-67, wherein the chemotherapeutic agent is selected from the group consisting of temozolomide, sorafenib, cisplatin, 5-fluorouracil and doxorubicin.

70. The method of claim 68, wherein the chemotherapeutic agent is temozolomide.

71. The method of claim 68, wherein the chemotherapeutic agent is sorafenib.

72. The method of claim 68, wherein the chemotherapeutic agent is cisplatin.

73. The method of claim 68, wherein the chemotherapeutic agent is 5-fluorouracil.

74. The method of claim 68, wherein the chemotherapeutic agent is doxorubicin.
